(19) United States
(12) Patent Application Publication
(10) Pub. No.: US 2023/0255970 A1
(43) Pub. Date: Aug. 17, 2023

(54) COMPOSITIONS AND METHODS FOR TREATING PROLIFERATIVE DISEASES

(71) Applicants: The Broad Institute, Inc., Cambridge, MA (US); The Trustees of the University of Pennsylvania, Philadelphia, PA (US)

(72) Inventors: Tzipora Sarah Karin EISINGER, Philadelphia, PA (US); Gabrielle CIOTTI, Philadelphia, PA (US); Ashley M. FULLER, Philadelphia, PA (US); Mohammad ROHBAN, Cambridge, MA (US); Anne Carpenter VAN DYK, Cambridge, MA (US); Shantanu SINGH, Cambridge, MA (US)

(73) Assignees: The Broad Institute, Inc., Cambridge, MA (US); The Trustees of the University of Pennsylvania, Philadelphia, PA (US)

(21) Appl. No.: 18/020,827

(22) PCT Filed: Aug. 12, 2021
(86) PCT No.: PCT/US2021/045694

§ 371 Date: Feb. 10, 2023

Related U.S. Application Data
(60) Provisional application No. 63/064,765, filed on Aug. 12, 2020.

(51) Int. Cl. A61K 31/517; A61P 35/00

(52) U.S. Cl. CPC A61K 31/517; A61P 35/00

(57) ABSTRACT

The present invention features compositions and methods for treating proliferative diseases such as cancer (e.g., sarcoma, pancreas, prostate, head and neck, liver, and breast cancer) that inhibit the growth of NF-κB and/or Hippo associated neoplasias.

### CROSS REFERENCE TO RELATED APPLICATIONS

This application is the U.S. National Stage Application, pursuant to 35 U.S.C. § 371 of PCT International Application No. PCT/US2021/045694, filed Aug. 12, 2021, designating the United States and published in English, which claims priority to and the benefit of U.S. App. No. 63/064,765, filed Aug. 12, 2020, the entire contents of each of which are hereby incorporated by reference in their entirety.

### STATEMENT OF RIGHTS TO INVENTIONS MADE UNDER FEDERALLY SPONSORED RESEARCH

This invention was made with government support under Grant No. R35 GM122547 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Sep. 22, 2021, is named 167741_023801PCT_SL.txt and is 9,949 bytes in size.

### BACKGROUND OF THE INVENTION

Despite improvements in treating cancer and other proliferative diseases, there remains a critical need for targeted therapies for the treatment or prophylaxis of such diseases. The Hippo pathway and nuclear factor kappa-light-chain-enhancer of activated B cells (NF-κB) signaling are critical regulators of cell survival and differentiation, and dysregulation of these pathways is implicated in a number of cancers. The Hippo pathway includes the transcriptional effectors, Yes-associated protein (YAP1) and Transcriptional co-activator with a PDZ-domain (TAZ). Over the last 15 years, the NF-κB pathway has emerged as a critical driver of tumorigenesis, independent of mutant oncogene status, in sarcomas, as well as pancreas, prostate, head and neck, liver, and breast cancer. Currently, there are no commercially available specific inhibitors of NF-κB with the necessary cell permeability, target specificity, and efficacy for clinical use. Improved compositions and methods for treating or preventing proliferative disease associated with these pathways are urgently required.

### SUMMARY

As described below, the present invention features compositions and methods for treating proliferative diseases, such as cancer (e.g., sarcoma, pancreas, prostate, head and neck, breast, liver) that inhibit the growth of NF-κB and/or Hippo pathway associated neoplasias.

In one aspect, the disclosure provides a method for inhibiting proliferation or survival of a neoplasia associated with an NF-κB pathway in a cell, the method involving contacting the cell with a compound selected from the group containing N-[[(8R,9S)-6-[(2R)-1-hydroxypropan-2-yl]-8-methyl-5-oxo-10-oxa-1,6,13,14 tetrazabicyclo[10.2.1]pentadeca-12(15),13-dien-9-yl]methyl]-N-methyl-4 phenoxybenzenesulfonamide, (4S,5R)-5-((dimethylamino)methyl)-2-((R)-1-hydroxypropan-2-yl)-4-methyl-8-(pyridin-2-ylethynyl)-2,3,4,5-tetrahydrobenzo[b][1,4,5]oxathiazocine 1,1-dioxide, N-[(4S,7S,8S)-8-methoxy-4,7,10-trimethyl-11-oxo-2-oxa-5,10-diazabicyclo[10.4.0]hexadeca-1(12),13,15-trien-15-yl]-4-phenylbenzamide, 1-[(4R,7S,8S)-8-methoxy-4,7,10-trimethyl-11-oxo-2-oxa-5,10-diazabicyclo[10.4.0]hexadeca-1(12),13,15-trien-14-yl]-3-[4-(trifluoromethyl)phenyl]urea, 2-[(3S,6aR,8S,10aR)-3-hydroxy-1-(3-methoxyphenyl)sulfonyl-3,4,6,6a,8,9,10,10a-octahydro-2H-pyrano[2,3-c][1,5]oxazocin-8-yl]-1-(4-phenyl-1-piperazinyl)ethanone, 1-pyridin-4-yl-3-(2,4,6-trichlorophenyl)urea, 4-(5,7,7,10,10-pentamethyl-8,9-dihydronaphtho[2,3-b][1,4]benzodiazepin-13-yl)benzoic acid (LE-135), 1-(3,3a,4,5,6,6a-hexahydro-1H-cyclopenta[c]pyrrol-2-yl)-3-(4-methylphenyl)sulfonylurea, 5-(1,4-diazepan-1-ylsulfonyl)-2H-isoquinolin-1-one, 1-[(3,4-dimethoxyphenyl)methyl]-6,7-dimethoxyisoquinoline, 5-(4-chlorophenyl)-6-ethylpyrimidine-2,4-diamine, 7-hydroxy-3-(4-hydroxyphenyl)chromen-4-one, N-benzylquinazolin-4-amine, (2R,3R,3aS,9bS)-7-(1-cyclohexenyl)-N-(cyclopropylmethyl)-3-(hydroxymethyl)-6-oxo-1,2,3,3a,4,9b-hexahydropyrrolo[2,3-a]indolizine-2-carboxamide, N-[(1R,3R,4aS,9aR)-3-[2-[(3-fluorophenyl)methylamino]-2-oxoethyl]-1-(hydroxymethyl)-3,4,4a,9a-tetrahydro-1H-pyrano[3,4-b]benzofuran-6-yl]-1,3-benzodioxole-5-carboxamide, (1S,9R,10R,11R)-11-N-ethyl-10-(hydroxymethyl)-5-(2-methoxyphenyl)-6-oxo-12-N-propyl-7,12-diazatricyclo[7.2.1.02,7]dodeca-2,4-diene-11,12-dicarboxamide, N-[(1S,3S,4aR,9aS)-1-(hydroxymethyl)-3-[2-oxo-2-(1-piperidinyl)ethyl]-3,4,4a,9a-tetrahydro-1H-pyrano[3,4-b]benzofuran-6-yl]-4-oxanecarboxamide, N-[(5S,6S,9S)-8-(cyclopropylmethyl)-5-methoxy-3,6,9-trimethyl-2-oxo-11-oxa-3,8-diazabicyclo[10.4.0]hexadeca-1(12),13,15-trien-14-yl]-2-fluorobenzamide, N-[(4R,7S,8R)-8-methoxy-4,7,10-trimethyl-11-oxo-5-(1,3-thiazol-2-ylmethyl)-2-oxa-5,10-diazabicyclo[10.4.0]hexadeca-1(12),13,15-trien-14-yl]cyclohexanecarboxamide, 2-[(3S,6aR,8R,10aR)-1-(1,3-benzodioxol-5-ylmethyl)-3-hydroxy-3,4,6,6a,8,9,10,10a-octahydro-2H-pyrano[2,3-c][1,5]oxazocin-8-yl]-1-piperidin-1-ylethanone, 2-[(1R,3R,4aS,9aR)-1-(hydroxymethyl)-6-[(3-methoxyphenyl)sulfonylamino]-3,4,4a,9a-tetrahydro-TH-pyrano[3,4-b]benzofuran-3-yl]acetic acid methyl ester, 4-fluoro-N-[(2R,3R)-5-[(2R)-1-hydroxypropan-2-yl]-3-methyl-2-(methylaminomethyl)-6-oxo-3,4-dihydro-2H-1,5-benzoxazocin-10-yl]benzenesulfonamide, N-[(2S,3S,6R)-2-(hydroxymethyl)-6-[2-oxo-2-(1,3-thiazol-2-ylamino)ethyl]oxan-3-yl]-3-piperidin-1-ylpropanamide, N-[(4S,7R,8R)-8-methoxy-4,7,10-trimethyl-11-oxo-5-(phenylmethyl)-2-oxa-5,10-diazabicyclo[10.4.0]hexadeca-1(12),13,15-trien-14-yl]butanamide, 2-[(2R,3R,6S)-3-[[(2,5-difluoroanilino)-oxomethyl]amino]-2-(hydroxymethyl)-3,6-dihydro-2H-pyran-6-yl]-N-[3-(4-morpholinyl)propyl]acetamide, N-benzyl-2-chloroquinazolin-4-amine, N-[(2R,3S,6S)-6-[2-[(4-fluorophenyl)sulfonylamino]ethyl]-2-(hydroxymethyl)oxan-3-yl]oxane-4-carboxamide, 5-amino-1-[2,6-dichloro-4-(trifluoromethyl)phenyl]-4-(trifluoromethane)sulfinyl-1H-pyrazole-3-carbonitrile, N-[(1S,3S,4aS,9aR)-1-(hydroxymethyl)-3-[2-oxo-2-(pyridin-2-ylmethylamino)ethyl]-3,4,4a,9a-tetrahydro-1H-pyrano[3,4-b][1]benzofuran-6-yl]cyclobutanecarboxamide, and 1-[[(8S,9R)-6-[(2S)-1-hydroxypropan-2-yl]-8-methyl-5-oxo-10-oxa-1,6,14,15-tetrazabicyclo[10.3.0]pentadeca-12,14-dien-9-yl]methyl]-1-methyl-3-(3-pyridin-2-yloxyphenyl)urea. In some embodiments, the cell overexpresses peroxisome proliferator-activated receptor gamma coactivator 1-alpha (PGC-1α).

In another aspect, the invention provides a method for inhibiting proliferation or survival of a neoplasia associated with an NF-κB pathway in a cell, the method involving contacting the cell with a compound selected from the group containing N-benzylquinazolin-4-amine, N-[(4S,7S,8S)-8-methoxy-4,7,10-trimethyl-11-oxo-2-oxa-5,10-diazabicyclo[10.4.0]hexadeca-1(12),13,15-trien-15-yl]-4-phenylbenzamide, and 1-[(4R,7S,8S)-8-methoxy-4,7,10-trimethyl-11-oxo-2-oxa-5,10-diazabicyclo[10.4.0]hexadeca-1(12),13,15-trien-14-yl]-3-[4-(trifluoromethyl)phenyl]urea. In some embodiments, the cell overexpresses peroxisome proliferator-activated receptor gamma coactivator 1-alpha (PGC-1α).

In another aspect, the invention provides a method for inhibiting proliferation or survival of a neoplasia associated with an NF-κB pathway in a cell, the method involving contacting the cell with an effective amount of N-benzylquinazolin-4-amine. In some embodiments, the cell overexpresses peroxisome proliferator-activated receptor gamma coactivator 1-alpha (PGC-1α).

In various embodiments of the previous aspects, or any other aspect of the invention delineated herein, the neoplasia is selected from the group containing sarcoma, pancreatic cancer, prostate cancer, head and neck cancer, breast cancer, and liver cancer.

In various embodiments of the previous aspects, or any other aspect of the invention delineated herein, the cell is a mammalian cell.

In various embodiments of the previous aspects, or any other aspect of the invention delineated herein, the cell is in vitro or in vivo.

The present disclosure includes methods of treating a neoplasia associated with the NF-κB and/or Hippo pathway in a subject, the method comprising administering to the subject a compound selected from the group consisting of:
(2S,3S,4R)-1-[2-(dimethylamino)acetyl]-4-(hydroxymethyl)-3-[4-(2-methoxyphenyl)phenyl]azetidine-2-carbonitrile,
(1S,9R,10R,11R)-11-N-ethyl-10-(hydroxymethyl)-5-(2-methoxyphenyl)-6-oxo-12-N-propyl-7,12-diazatricyclo[7.2.1.02,7]dodeca-2,4-diene-11,12-dicarboxamide,
(2R,3R,3aS,9bS)-7-(1-cyclohexenyl)-N-(cyclopropylmethyl)-3-(hydroxymethyl)-6-oxo-1,2,3,3a,4,9b-hexahydropyrrolo[2,3-a]indolizine-2-carboxamide,
(3S)-2-[(S)-tert-butylsulfinyl]-3-(2-hydroxyethyl)-N-[(3-methoxyphenyl)methyl]-4-(3-pyridin-4-ylphenyl)-1,3-dihydropyrrolo[3,4-c]pyridine-6-carboxamide,
(4S,5R)-5-(((cyclopropylmethyl)(methyl)amino) methyl)-8-(4-((3-fluorophenyl) ethynyl)phenyl)-2-((S)-1-hydroxypropan-2-yl)-4-methyl-2,3,4,5-tetrahydrobenzo[b][1,4,5]oxathiazocine 1,1-dioxide,
(4S,5R)-5-((dimethylamino)methyl)-2-((R)-1-hydroxypropan-2-yl)-4-methyl-8-(pyridin-2-ylethynyl)-2,3,4,5-tetrahydrobenzo[b][1,4,5]oxathiazocine 1,1-dioxide,
[(1S,2S,6R,10S,11R,13S,14R,15R)-13-acetyloxy-1,6-dihydroxy-8-(hydroxymethyl)-4,12,12,15-tetramethyl-5-oxo-14-tetracyclo[8.5.0.02,6.011,13]pentadeca-3,8-dienyl] tetradecanoate, 1-(2,4-dichlorophenyl)-6-methyl-N-piperidin-1-yl-4H-indeno[1,2-c] pyrazole-3-carboxamide, 1-(3,3a,4,5,6,6a-hexahydro-1H-cyclopenta[c]pyrrol-2-yl)-3-(4-methylphenyl)sulfonylurea, 1-[(3,4-dimethoxyphenyl)methyl]-6,7-dimethoxyisoquinoline,
1-[(4R,7S,8S)-8-methoxy-4,7,10-trimethyl-11-oxo-2-oxa-5,10-diazabicyclo[10.4.0]hexadeca-1(12),13,15-trien-14-yl]-3-[4-(trifluoromethyl)phenyl]urea,
1-[[(10R,11S)-13-[(2R)-1-hydroxypropan-2-yl]-11-methyl-14-oxo-9-oxa-13-azatricyclo[13.4.0.02,7] nonadeca-1(19),2,4,6,15,17-hexaen-10-yl]methyl]-3-(2-methoxyphenyl)-1-methylurea,
1-[[(10R,11S)-13-[(2R)-1-hydroxypropan-2-yl]-11-methyl-14-oxo-9-oxa-13-azatricyclo[13.4.0.02,7]nonadeca-1(19),2,4,6,15,17-hexaen-10-yl] methyl]-3-(2-methoxyphenyl)-1-methylurea,
1-[[(8S,9R)-6-[(2S)-1-hydroxypropan-2-yl]-8-methyl-5-oxo-10-oxa-1,6,14,15-tetrazabicyclo[10.3.0]pentadeca-12,14-dien-9-yl]methyl]-1-methyl-3-(3-pyridin-2-yloxyphenyl)urea,
1-butyl-3-(3-hydroxypropyl)-8-(3-tricyclo[3.3.1.03,7]nonanyl)-7H-purine-2,6-dione, 1-tert-butyl-3-(4-chlorophenyl)pyrazolo[3,4-d]pyrimidin-4-amine,
2-[(1R,3R,4aS,9aR)-1-(hydroxymethyl)-6-[(3-methoxyphenyl)sulfonylamino]-3,4,4a,9a-tetrahydro-1H-pyrano[3,4-b]benzofuran-3-yl]acetic acid methyl ester,
2-[(2R,3R,6S)-3-[[(2,5-difluoroanilino)-oxomethyl]amino]-2-(hydroxymethyl)-3,6-dihydro-2H-pyran-6-yl]-N-[3-(4-morpholinyl)propyl]acetamide,
2-[(3S,6aR,8R,10aR)-1-(1,3-benzodioxol-5-ylmethyl)-3-hydroxy-3,4,6,6a,8,9,10,10a-octahydro-2H-pyrano[2,3-c][1,5]oxazocin-8-yl]-1-piperidin-1-ylethanone,
2-[(3S,6aR,8S,10aR)-3-hydroxy-1-(3-methoxyphenyl)sulfonyl-3,4,6,6a,8,9,10,10a-octahydro-2H-pyrano[2,3-c][1,5]oxazocin-8-yl]-1-(4-phenyl-1-piperazinyl)ethanone1-pyridin-4-yl-3-(2,4,6-trichlorophenyl)urea,
2-chloro-5-nitro-N-phenylbenzamide,
3-((4S,5S)-5-(((benzo[d][1,3]dioxol-5-ylmethyl)(methyl)amino)methyl)-2-((R)-1-hydroxypropan-2-yl)-4-methyl-1,1-dioxido-2,3,4,5-tetrahydrobenzo[b][1,4,5]oxathiazocin-8-yl)-N,N-dimethylbenzamide,
3-chloro-N-((2R,3R)-4-((4-chloro-N-methylphenyl)sulfonamido)-3-methoxy-2-methylbutyl)-N-((S)-1-hydroxypropan-2-yl)benzenesulfonamide,
3-chloro-N-[(2R,3R)-4-[(4-chlorophenyl)sulfonyl-methylamino]-3-methoxy-2-methylbutyl]-N-[(2R)-1-hydroxypropan-2-yl]benzenesulfonamide,
4-(5,7,7,10,10-pentamethyl-8,9-dihydronaphtho[2,3-b][1,4]benzodiazepin-13-yl)benzoic acid (LE-135),
4-(5-pyridin-2-yl-1H-pyrazol-4-yl)quinoline,
4-[4-(1,3-benzodioxol-5-yl)-5-pyridin-2-yl-1H-imidazol-2-yl]benzamide,
4-[4-(2,3-dihydro-1,4-benzodioxin-6-yl)-5-methyl-1H-pyrazol-3-yl]-6-ethylbenzene-1,3-diol, 4-fluoro-N-[(2R,3R)-5-[(2R)-1-hydroxypropan-2-yl]-3-methyl-2-(methylaminomethyl)-6-oxo-3,4-dihydro-2H-1,5-benzoxazocin-10-yl]benzenesulfonamide,
5-((7-(benzyloxy)quinazolin-4-yl)amino)-4-fluoro-2-methylphenol,
5-(1,4-diazepan-1-ylsulfonyl)-2H-isoquinolin-1-one,
5-(4-chlorophenyl)-6-ethylpyrimidine-2,4-diamine,
5-amino-1-[2,6-dichloro-4-(trifluoromethyl)phenyl]-4-(trifluoromethane)sulfinyl-H-pyrazole-3-carbonitrile,
5-methyl-2-phenyl-4H-pyrazol-3-one,
7-hydroxy-3-(4-hydroxyphenyl)chromen-4-one,
methyl 3-[3-[2-(2-carbamoylphenoxy)acetyl]-2,5-dimethylpyrrol-1-yl]propanoate, N-(((4R,5R)-2-((R)-1-hydroxypropan-2-yl)-4-methyl-1,1-dioxido-8-(pent-1-yn-1-yl)-2,3,4,5-tetrahydrobenzo[b][1,4,5]oxathiazocin-5-yl)methyl)-3-methoxy-N-methylbenzenesulfonamide,
N-[(1R,3R,4aS,9aR)-3-[2-[(3-fluorophenyl)methylamino]-2-oxoethyl]-1-(hydroxymethyl)-3,4,4a,9a-tetrahydro-1H-pyrano[3,4-b]benzofuran-6-yl]-1,3-benzodioxole-5-carboxamide,
N-[(1S,3S,4aR,9aS)-1-(hydroxymethyl)-3-[2-oxo-2-(1-piperidinyl)ethyl]-3,4,4a,9a-tetrahydro-1H-pyrano[3,4-b]benzofuran-6-yl]-4-oxanecarboxamide,
N-[(1S,3S,4aS,9aR)-1-(hydroxymethyl)-3-[2-oxo-2-(pyridin-2-ylmethylamino)ethyl]-3,4,4a,9a-tetrahydro-1H-pyrano[3,4-b][1]benzofuran-6-yl]cyclobutanecarboxamide,
N-[(2R,3S,6S)-6-[2-[(4-fluorophenyl)sulfonylamino]ethyl]-2-(hydroxymethyl)oxan-3-yl]oxane-4-carboxamide,
N-[(2S,3S,6R)-2-(hydroxymethyl)-6-[2-oxo-2-(1,3-thiazol-2-ylamino)ethyl]oxan-3-yl]-3-piperidin-1-ylpropanamide,
N-[(3R,9S,10R)-12-[(2S)-1-hydroxypropan-2-yl]-3,10-dimethyl-9-(methylaminomethyl)-13-oxo-2,8-dioxa-12-azabicyclo[12.4.0]octadeca-1(14),15,17-trien-16-yl]cyclohexanecarboxamide,
N-[(4R,7S,8R)-8-methoxy-4,7,10-trimethyl-11-oxo-5-(1,3-thiazol-2-ylmethyl)-2-oxa-5,10-diazabicyclo[10.4.0]hexadeca-1(12),13,15-trien-14-yl]cyclohexanecarboxamide,
N-[(4S,7R,8R)-8-methoxy-4,7,10-trimethyl-11-oxo-5-(phenylmethyl)-2-oxa-5,10-diazabicyclo[10.4.0]hexadeca-1(12),13,15-trien-14-yl]butanamide,
N-[(4S,7S,8S)-8-methoxy-4,7,10-trimethyl-11-oxo-2-oxa-5,10-diazabicyclo[10.4.0]hexadeca-1(12),13,15-trien-15-yl]-4-phenylbenzamide,
N-[(5S,6S,9S)-8-(cyclopropylmethyl)-5-methoxy-3,6,9-trimethyl-2-oxo-11-oxa-3,8-diazabicyclo[10.4.0]hexadeca-1(12),13,15-trien-14-yl]-2-fluorobenzamide,
N-[[(2R,3R)-8-bromo-5-[(2R)-1-hydroxypropan-2-yl]-3-methyl-6-oxo-3,4-dihydro-2H-pyrido[2,3-b][1,5]oxazocin-2-yl]methyl]-2-methoxy-N-methylacetamide,
N-[[(8R,9S)-6-[(2R)-1-hydroxypropan-2-yl]-8-methyl-5-oxo-10-oxa-1,6,13,14 tetrazabicyclo[10.2.1]pentadeca-12(15),13-dien-9-yl]methyl]-N-methyl-4 phenoxybenzenesulfonamide,
N-benzyl-2-chloroquinazolin-4-amine,
N-benzylquinazolin-4-amine; or
   a pharmaceutically acceptable salt thereof, or a tautomer, stereoisomer, prodrug and/or solvate of any of the foregoing. The method of treating a neoplasia associated with the NF-κB and/or Hippo pathway in a subject, the method may comprise administering to the subject a compound selected from the group consisting of N-benzylquinazolin-4-amine, N-[(4S,7S,8S)-8-methoxy-4,7,10-trimethyl-11-oxo-2-oxa-5,10-diazabicyclo[10.4.0]hexadeca-1(12),13,15-trien-15-yl]-4-phenylbenzamide, and 1-[(4R,7S,8S)-8-methoxy-4,7,10-trimethyl-11-oxo-2-oxa-5,10-diazabicyclo[10.4.0]hexadeca-1(12),13,15-trien-14-yl]-3-[4-(trifluoromethyl)phenyl]urea; or
   a pharmaceutically acceptable salt thereof, or a tautomer, stereoisomer, prodrug and/or solvate of any of the foregoing. In certain implementations, the method of treating a neoplasia associated with the NF-κB and/or Hippo pathway in a subject, the method comprising administering to the subject N-benzylquinazolin-4-amine.

In another aspect, the invention provides a method of treating a neoplasia associated with the NF-κB pathway in a subject, the method involving administering to the subject a compound selected from the group containing N-[[(8R,9S)-6-[(2R)-1-hydroxypropan-2-yl]-8-methyl-5-oxo-10-oxa-1,6,13,14 tetrazabicyclo[10.2.1]pentadeca-12(15),13-dien-9-yl]methyl]-N-methyl-4 phenoxybenzenesulfonamide, (4S,5R)-5-((dimethylamino)methyl)-2-((R)-1-hydroxypropan-2-yl)-4-methyl-8-(pyridin-2-ylethynyl)-2,3,4,5-tetrahydrobenzo[b][1,4,5]oxathiazocine 1,1-dioxide, N-[(4S,7S,8S)-8-methoxy-4,7,10-trimethyl-11-oxo-2-oxa-5,10-diazabicyclo[10.4.0]hexadeca-1(12),13,15-trien-15-yl]-4-phenylbenzamide, 1-[(4R,7S,8S)-8-methoxy-4,7,10-trimethyl-11-oxo-2-oxa-5,10-diazabicyclo[10.4.0]hexadeca-1(12),13,15-trien-14-yl]-3-[4-(trifluoromethyl)phenyl]urea, 2-[(3S,6aR,8S,10aR)-3-hydroxy-1-(3-methoxyphenyl)sulfonyl-3,4,6,6a,8,9,10,10a-octahydro-2H-pyrano[2,3-c][1,5]oxazocin-8-yl]-1-(4-phenyl-1-piperazinyl)ethanone, 1-pyridin-4-yl-3-(2,4,6-trichlorophenyl)urea, 4-(5,7,7,10,10-pentamethyl-8,9-dihydronaphtho[2,3-b][1,4]benzodiazepin-13-yl)benzoic acid (LE-135), 1-(3,3a,4,5,6,6a-hexahydro-1H-cyclopenta[c]pyrrol-2-yl)-3-(4-methylphenyl)sulfonylurea, 5-(1,4-diazepan-1-ylsulfonyl)-2H-isoquinolin-1-one, 1-[(3,4-dimethoxyphenyl)methyl]-6,7-dimethoxyisoquinoline, 5-(4-chlorophenyl)-6-ethylpyrimidine-2,4-diamine, 7-hydroxy-3-(4-hydroxyphenyl)chromen-4-one, N-benzylquinazolin-4-amine, (2R,3R,3aS,9bS)-7-(1-cyclohexenyl)-N-(cyclopropylmethyl)-3-(hydroxymethyl)-6-oxo-1,2,3,3a,4,9b-hexahydropyrrolo[2,3-a]indolizine-2-carboxamide, N-[(1R,3R,4aS,9aR)-3-[2-[(3-fluorophenyl)methylamino]-2-oxoethyl]-1-(hydroxymethyl)-3,4,4a,9a-tetrahydro-1H-pyrano[3,4-b]benzofuran-6-yl]-1,3-benzodioxole-5-carboxamide, (1S,9R,10R,11R)-11-N-ethyl-10-(hydroxymethyl)-5-(2-methoxyphenyl)-6-oxo-12-N-propyl-7,12-diazatricyclo[7.2.1.02,7]dodeca-2,4-diene-11,12-dicarboxamide, N-[(1S,3S,4aR,9aS)-1-(hydroxymethyl)-3-[2-oxo-2-(1-piperidinyl)ethyl]-3,4,4a,9a-tetrahydro-1H-pyrano[3,4-b]benzofuran-6-yl]-4-oxanecarboxamide, N-[(5S,6S,9S)-8-(cyclopropylmethyl)-5-methoxy-3,6,9-trimethyl-2-oxo-11-oxa-3,8-diazabicyclo[10.4.0]hexadeca-1(12),13,15-trien-14-yl]-2-fluorobenzamide, N-[(4R,7S,8R)-8-methoxy-4,7,10-trimethyl-11-oxo-5-(1,3-thiazol-2-ylmethyl)-2-oxa-5,10-diazabicyclo[10.4.0]hexadeca-1(12),13,15-trien-14-yl]cyclohexanecarboxamide, 2-[(3S,6aR,8R,10aR)-1-(1,3-benzodioxol-5-ylmethyl)-3-hydroxy-3,4,6,6a,8,9,10,10a-octahydro-2H-pyrano[2,3-c][1,5]oxazocin-8-yl]-1-piperidin-1-ylethanone, 2-[(1R,3R,4aS,9aR)-1-(hydroxymethyl)-6-[(3-methoxyphenyl)sulfonylamino]-3,4,4a,9a-tetrahydro-1H-pyrano[3,4-b]benzofuran-3-yl]acetic acid methyl ester, 4-fluoro-N-[(2R,3R)-5-[(2R)-1-hydroxypropan-2-yl]-3-methyl-2-(methylaminomethyl)-6-oxo-3,4-dihydro-2H-1,5-benzoxazocin-10-yl]benzenesulfonamide, N-[(2S,3S,6R)-2-(hydroxymethyl)-6-[2-oxo-2-(1,3-thiazol-2-ylamino)ethyl]oxan-3-yl]-3-piperidin-1-ylpropanamide, N-[(4S,7R,8R)-8-methoxy-4,7,10-trimethyl-11-oxo-5-(phenylmethyl)-2-oxa-5,10-diazabicyclo[10.4.0]hexadeca-1(12),13,15-trien-14-yl]butanamide, 2-[(2R,3R,6S)-3-[[(2,5-difluoroanilino)-oxomethyl]amino]-2-(hydroxymethyl)-3,6-dihydro-2H-pyran-6-yl]-N-[3-(4-morpholinyl)propyl]acetamide, N-benzyl-2-chloroquinazolin-4-amine, N-[(2R,3S,6S)-6-[2-[(4-fluorophenyl)sulfonylamino]ethyl]-2-(hydroxymethyl)oxan-3-yl]oxane-4-carboxamide, 5-amino-1-[2,6-dichloro-4-(trifluoromethyl)phenyl]-4-(trifluoromethane)sulfinyl-1H-pyrazole-3-carbonitrile, N-[(1S,3S,4aS,9aR)-1-(hydroxymethyl)-3-[2-oxo-2-(pyridin-2-ylmethylamino)ethyl]-3,4,4a,9a-tetrahydro-1H-pyrano[3,4-b][1]benzofuran-6-yl]cyclobutanecarboxamide, and 1-[[(8S,9R)-6-[(2S)-1-hydroxypropan-2-yl]-8-methyl-5-oxo-10-oxa-1,6,14,15-tetrazabicyclo[10.3.0]pentadeca-12,14-dien-9-yl]methyl]-1-methyl-3-(3-pyridin-2-yloxyphenyl)urea.

In another aspect, the invention provides a method of treating a neoplasia associated with the NF-κB pathway in a subject, the method involving administering to the subject a compound selected from the group containing N-benzylquinazolin-4-amine, N-[(4S,7S,8S)-8-methoxy-4,7,10-trimethyl-11-oxo-2-oxa-5,10-diazabicyclo[10.4.0]hexadeca-1(12),13,15-trien-15-yl]-4-phenylbenzamide, and 1-[(4R,7S,8S)-8-methoxy-4,7,10-trimethyl-11-oxo-2-oxa-5,10-diazabicyclo[10.4.0]hexadeca-1(12),13,15-trien-14-yl]-3-[4-(trifluoromethyl)phenyl]urea.

In another aspect, the invention provides a method of treating a neoplasia associated with the NF-κB pathway in a subject, the method involving administering to the subject N-benzylquinazolin-4-amine.

Methods are also provided for inhibiting proliferation or survival of a neoplasia associated with a Hippo pathway in a cell comprising contacting the cell with a compound selected from:
N-[[(8R,9S)-6-[(2R)-1-hydroxypropan-2-yl]-8-methyl-5-oxo-10-oxa-1,6,13,14 tetrazabicyclo[10.2.1]pentadeca-12(15),13-dien-9-yl]methyl]-N-methyl-4 phenoxybenzenesulfonamide,
(4S,5R)-5-((dimethylamino)methyl)-2-((R)-1-hydroxypropan-2-yl)-4-methyl-8-(pyridin-2-ylethynyl)-2,3,4,5-tetrahydrobenzo[b][1,4,5]oxathiazocine 1,1-dioxide,
N-[(4S,7S,8S)-8-methoxy-4,7,10-trimethyl-11-oxo-2-oxa-5,10-diazabicyclo[10.4.0]hexadeca-1(12),13,15-trien-15-yl]-4-phenylbenzamide,
1-[(4R,7S,8S)-8-methoxy-4,7,10-trimethyl-11-oxo-2-oxa-5,10-diazabicyclo[10.4.0]hexadeca-1(12),13,15-trien-14-yl]-3-[4-(trifluoromethyl)phenyl]urea,
2-[(3S,6aR,8S,10aR)-3-hydroxy-1-(3-methoxyphenyl)sulfonyl-3,4,6,6a,8,9,10,10a-octahydro-2H-pyrano[2,3-c][1,5]oxazocin-8-yl]-1-(4-phenyl-1-piperazinyl)ethanone, 1-pyridin-4-yl-3-(2,4,6-trichlorophenyl)urea,
4-(5,7,7,10,10-pentamethyl-8,9-dihydronaphtho[2,3-b][1,4]benzodiazepin-13-yl)benzoic acid (LE-135),
1-(3,3a,4,5,6,6a-hexahydro-TH-cyclopenta[c]pyrrol-2-yl)-3-(4-methylphenyl)sulfonylurea,
5-(1,4-diazepan-1-ylsulfonyl)-2H-isoquinolin-1-one,
1-[(3,4-dimethoxyphenyl)methyl]-6,7-dimethoxyisoquinoline,
5-(4-chlorophenyl)-6-ethylpyrimidine-2,4-diamine,
7-hydroxy-3-(4-hydroxyphenyl)chromen-4-one,
N-benzylquinazolin-4-amine,
(2R,3R,3aS,9bS)-7-(1-cyclohexenyl)-N-(cyclopropylmethyl)-3-(hydroxymethyl)-6-oxo-1,2,3,3a,4,9b-hexahydropyrrolo[2,3-a]indolizine-2-carboxamide,
N-[(1R,3R,4aS,9aR)-3-[2-[(3-fluorophenyl)methylamino]-2-oxoethyl]-1-(hydroxymethyl)-3,4,4a,9a-tetrahydro-1H-pyrano[3,4-b]benzofuran-6-yl]-1,3-benzodioxole-5-carboxamide,
(1S,9R,10R,11R)-11-N-ethyl-10-(hydroxymethyl)-5-(2-methoxyphenyl)-6-oxo-12-N-propyl-7,12-diazatricyclo[7.2.1.02,7]dodeca-2,4-diene-11,12-dicarboxamide,
N-[(1S,3S,4aR,9aS)-1-(hydroxymethyl)-3-[2-oxo-2-(1-piperidinyl)ethyl]-3,4,4a,9a-tetrahydro-1H-pyrano[3,4-b]benzofuran-6-yl]-4-oxanecarboxamide,
N-[(5S,6S,9S)-8-(cyclopropylmethyl)-5-methoxy-3,6,9-trimethyl-2-oxo-11-oxa-3,8-diazabicyclo[10.4.0]hexadeca-1(12),13,15-trien-14-yl]-2-fluorobenzamide,
N-[(4R,7S,8R)-8-methoxy-4,7,10-trimethyl-11-oxo-5-(1,3-thiazol-2-ylmethyl)-2-oxa-5,10-diazabicyclo[10.4.0]hexadeca-1(12),13,15-trien-14-yl]cyclohexanecarboxamide,
2-[(3S,6aR,8R,10aR)-1-(1,3-benzodioxol-5-ylmethyl)-3-hydroxy-3,4,6,6a,8,9,10,10a-octahydro-2H-pyrano[2,3-c][1,5]oxazocin-8-yl]-1-piperidin-1-ylethanone,
2-[(1R,3R,4aS,9aR)-1-(hydroxymethyl)-6-[(3-methoxyphenyl)sulfonylamino]-3,4,4a,9a-tetrahydro-1H-pyrano[3,4-b]benzofuran-3-yl]acetic acid methyl ester,
4-fluoro-N-[(2R,3R)-5-[(2R)-1-hydroxypropan-2-yl]-3-methyl-2-(methylaminomethyl)-6-oxo-3,4-dihydro-2H-1,5-benzoxazocin-10-yl]benzenesulfonamide,
N-[(2S,3S,6R)-2-(hydroxymethyl)-6-[2-oxo-2-(1,3-thiazol-2-ylamino)ethyl]oxan-3-yl]-3-piperidin-1-ylpropanamide,
N-[(4S,7R,8R)-8-methoxy-4,7,10-trimethyl-II-oxo-5-(phenylmethyl)-2-oxa-5,10-diazabicyclo[10.4.0]hexadeca-1(12),13,15-trien-14-yl]butanamide,
2-[(2R,3R,6S)-3-[[(2,5-difluoroanilino)-oxomethyl]amino]-2-(hydroxymethyl)-3,6-dihydro-2H-pyran-6-yl]-N-[3-(4-morpholinyl)propyl]acetamide,
N-benzyl-2-chloroquinazolin-4-amine,
N-[(2R,3S,6S)-6-[2-[(4-fluorophenyl)sulfonylamino]ethyl]-2-(hydroxymethyl)oxan-3-yl]oxane-4-carboxamide,
5-amino-1-[2,6-dichloro-4-(trifluoromethyl)phenyl]-4-(trifluoromethane)sulfinyl-1H-pyrazole-3-carbonitrile,
N-[(1S,3S,4aS,9aR)-1-(hydroxymethyl)-3-[2-oxo-2-(pyridin-2-ylmethylamino)ethyl]-3,4,4a,9a-tetrahydro-1H-pyrano[3,4-b][1]benzofuran-6-yl]cyclobutanecarboxamide, and
1-[[(8S,9R)-6-[(2S)-1-hydroxypropan-2-yl]-8-methyl-5-oxo-10-oxa-1,6,14,15-tetrazabicyclo[10.3.0]pentadeca-12,14-dien-9-yl]methyl]-1-methyl-3-(3-pyridin-2-yloxyphenyl)urea; or
   a pharmaceutically acceptable salt thereof, or a tautomer, stereoisomer, prodrug and/or solvate of any of the foregoing;
   thereby inhibiting proliferation or survival of the cell. In some embodiments, the method for inhibiting proliferation or survival of a neoplasia associated with a Hippo pathway in a cell, may comprise contacting the cell with a compound selected from the group consisting of:
N-benzylquinazolin-4-amine,
N-[(4S,7S,8S)-8-methoxy-4,7,10-trimethyl-11-oxo-2-oxa-5,10-diazabicyclo[10.4.0]hexadeca-1(12),13,15-trien-15-yl]-4-phenylbenzamide,
and 1-[(4R,7S,8S)-8-methoxy-4,7,10-trimethyl-11-oxo-2-oxa-5,10-diazabicyclo[10.4.0]hexadeca-1(12),13,15-trien-14-yl]-3-[4-(trifluoromethyl)phenyl]urea; or
   a pharmaceutically acceptable salt thereof, or a tautomer, stereoisomer, prodrug and/or solvate of any of the foregoing. In certain implementations, the method may comprise contacting the cell with an effective amount of N-benzylquinazolin-4-amine; or
   a pharmaceutically acceptable salt thereof, or a tautomer, stereoisomer, prodrug and/or solvate of any of the foregoing. In some embodiments, the cell overexpresses peroxisome proliferator-activated receptor gamma coactivator 1-alpha (PGC-1α).

In various embodiments of the previous aspects, or any other aspect of the invention delineated herein, the neoplasia is selected from the group consisting of sarcoma, pancreatic cancer, prostate cancer, head and neck cancer, breast cancer, and liver cancer. In some embodiments, the neoplasia is bladder cancer. In some embodiments, the neoplasia is undifferentiated pleomorphic sarcoma.

The invention provides compositions and methods useful for treating proliferative diseases, such as cancer (e.g., sarcoma, pancreas, prostate, head and neck, liver, and breast cancer). Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Margham, The Harper Collins Dictionary of Biology (1991). As used herein, the following terms have the meanings ascribed to them below, unless specified otherwise.

By “morphological signature” is meant one or more features of cellular morphology that is associated with the condition of a cell. In one embodiment, a morphological signature reflects the health or disease state of a cell. In another embodiment, a morphological signature is altered in response to contact with an agent. Methods for characterizing a morphological signature are known in the art (See, e.g., Bray et al., Nat Protoc. 2016 September; 11(9): 1757-1774, incorporated herein by reference in its entirety, Rohban et al., eLife 2017; 6:e24060, incorporated herein by reference in its entirety, and Caicedo et al., Nat. Meth. 2017, 14, 849-863, incorporated herein by reference in its entirety, Rohban, M., et al. “Discovery of small molecule pathway regulators by image profile matching.” bioRxiv 2021), incorporated by reference in its entirety).

By “YAP1 (yes-associated protein 1) polypeptide,” also known as YAP, is meant a protein or fragment thereof having at least about 85% identity to NCBI Accession No. P46937 that regulates transcription. The sequence of an exemplary human YAP1 protein is provided below (SEQ ID NO: 1):

```
>sp|P46937|YAP1_HUMAN Transcriptional coactivator YAP1 OS = Homo sapiens
OX = 9606 GN = YAP1 PE= 1 SV = 2
                10 20 30 40 50
MDPGQQPPPQ PAPQGQGQPP SQPPQGQGPP SGPGQPAPAA TQAAPQAPPA
                60 70 80 90 100
GHQIVHVRGD SETDLEALFN AVMNPKTANV PQTVPMRLRK LPDSFFKPPE
              110 120 130 140 150
PKSHSROAST DAGTAGALTP QHVRAHSSPA SLQLGAVSPG TLTPTGVVSG
              160 170 180 190 200
PAATPTAQHL ROSSFEIPDD VPLPAGWEMA KTSSGQRYFL NHIDQTTTWQ
              210 220 230 240 250
DPRKAMLSQM NVTAPTSPPV QQNMMNSASG PLPDGWEQAM TQDGEIYYIN
              260 270 280 290 300
HKNKTTSWLD PRLDPRFAMN QRISQSAPVK QPPPLAPQSP QGGVMGGSNS
              310 320 330 340 350
NQQQQMRLQQ LQMEKERLRL KQQELLROAM RNINPSTANS PKCQELALRS
              360 370 380 390 400
QLPTLEQDGG TQNPVSSPGM SQELRTMTTN SSDPFLNSGT YHSRDESTDS
              410 420 430 440 450
GLSMSSYSVP RTPDDFLNSV DEMDTGDTIN QSTLPSQQNR FPDYLEAIPG
              460 470 480 490 500
TNVDLGTLEG DGMNIEGEEL MPSLQEALSS DILNDMESVL AATKLDKESF
LTWL
```

By “TAZ (transcriptional co-activator with a PDZ-domain) polypeptide” is meant a protein or fragment thereof having at least about 85% identity to NCBI Accession No. Q16635 that has transcriptional regulatory activity. The sequence of an exemplary human TAZ protein is provided below (SEQ ID NO: 2):

```
>sp|Q16635|TAZ_HUMAN Tafazzin OS = Homo sapiens OX = 9606 GN = TAZ
PE = 1 SV = 1
                10 20 30 40 50
MPLHVKWPFP AVPPLTWTLA SSVVMGLVGT YSCFWTKYMN HLTVHNREVL
                60 70 80 90 100
YELIEKRGPA TPLITVSNHQ SCMDDPHLWG ILKLRHIWNL KLMRWTPAAA
              110 120 130 140 150
DICFTKELHS HFFSLGKCVP VCRGAEFFQA ENEGKGVLDT GRHMPGAGKR
              160 170 180 190 200
REKGDGVYQK GMDFILEKLN HGDWVHIFPE GKVNMSSEFL RFKWGIGRLI
              210 220 230 240 250
AECHLNPIIL PLWHVGMNDV LPNSPPYFPR FGQKITVLIG KPFSALPVLE
              260 270 280 290
RLRAENKSAV EMRKALTDFI QEEFOHLKTQ AEQLHNHLQP GR
```

By “Yap1” or “Yap1 polynucleotide” is meant a nucleic acid molecule encoding a Yap1 polypeptide. An exemplary Yap1 polynucleotide sequence is provided at Genbank Accession No. NM_001130145.3, which is hereby incorporated by reference in its entirety.

By “Taz polynucleotide” is meant a nucleic acid sequence encoding a TAZ polypeptide. An exemplary Taz polynucleotide sequence is provided at Genbank Accession No. NM_000116.5, which is hereby incorporated by reference in its entirety.

By “Foxm1 (Forkhead box protein M1) poly nucleotide” is meant a nucleic acid sequence encoding a FOXM1 polypeptide. An exemplary Foxm1 polynucleotide sequence is provided at Genbank Accession No. NM_001243088.1, which is hereby incorporated by reference in its entirety.

By “Ccl2 (Chemokine (C-C motif) ligand 2) polynucleotide” is meant a nucleic acid sequence encoding the small cytokine CCL2. An exemplary Ccl2 polynucleotide sequence is provided at Genbank Accession No. NM_0029182.4, which is hereby incorporated by reference in its entirety.

By “Hbegf (heparin binding EGF like growth factor) polynucleotide” is meant a nucleic acid sequence encoding an HBEGF polypeptide. An exemplary Hbegf polynucleotide sequence is provided at Genbank Accession No. NM_0019145.3, which is hereby incorporated by reference in its entirety.

By “Birc5 (baculoviral inhibitor of apoptosis repeat-containing 5) polynucleotide” is meant a nucleic acid sequence encoding a BIRC5 polypeptide. An exemplary Birc5 polynucleotide sequence is provided at Genbank Accession No. NM_001012270,2, which is hereby incorporated by reference in its entirety.

By “Rela (v-rel avian reticuloendotheliosis viral oncogene homolog A) polynucleotide” is meant a nucleic acid sequence encoding a RELA, or p65, polypeptide. An exemplary Rela polynucleotide sequence is provided at Genbank Accession No. NM_001145138.2, which is hereby incorporated by reference in its entirety.

By “Hippo pathway” is meant the signal cascade that may be initiated by phosphorylation and affects the localization and/or transcriptional activity of Yap1/Taz. Components of the Hippo pathway include mammalian Ste20-like kinases 1/2 (MST1/2) and large tumor suppressor 1/2 (LATS1/2), yes association protein (YAP1) and/or its paralog TAZ.

By “Hprt (Hypoxanthine-guanine phosphoribosyltransferase) polynucleotide” is meant a nucleic acid sequence encoding a HPRT polypeptide. An exemplary Hprt polynucleotide sequence is provided at Genbank Accession No. NM_000194, which is hereby incorporated by reference in its entirety.

By “K167 polynucleotide” is meant a nucleic acid sequence encoding a Ki67 polypeptide, a nuclear protein associated with proliferation. An exemplary Ki67 polynucleotide sequence is provided at Genbank Accession No. NM_001145966.2 which is hereby incorporated by reference in its entirety.

By PHLDA1 (Pleckstrin homology-like domain family A member 1) polynucleotide” is meant a nucleic acid sequence encoding a PHLDA1 polypeptide. An exemplary PHLDA1 polynucleotide sequence is provided at Genbank Accession No. NM_007350.3, which is hereby incorporated by reference in its entirety.

By IER2 (Immediate early response 2) polynucleotide” is meant a nucleic acid sequence encoding an IER2 polypeptide. An exemplary IER2 polynucleotide sequence is provided at Genbank Accession No. NM_004937.3, which is hereby incorporated y reference in its entirety.

By LITAF (Lipopolysaccharide-induced tumor necrosis factor-alpha factor) polynucleotide” is meant a nucleic acid sequence encoding a LITAF polypeptide. An exemplary LITAF polynucleotide sequence is provided at Genbank Accession No. NM_001136472.1, which is hereby incorporated by reference in its entirety.

By “NF-κB,” also known as “nuclear factor kappa-light-chain-enhancer of activated B cells,” is meant a protein complex that controls transcription of DNA, cytokine production and/or cell survival. In particular, Nuclear factor-κB (NF-κB) is a family of five master transcription factors, including NF-κBT/p105, NF-κB2/p100, RelA/p65, RelB and c-Rel, which can form various heterodimers or homodimers and bind to consensus DNA sequences at promoter regions of responsive genes.

By “NF-κB pathway associated neoplasia” is meant a cancer or other proliferative disorder whose growth, proliferation, or survival is associated with an alteration in NF-κB transcriptional regulation.

By “Hippo pathway associated neoplasia” is meant a cancer or other proliferative disorder whose growth, proliferation, or survival is associated with an alteration in Hippo pathway signaling results in abnormally functioning cells and, potentially tumorigenesis. For example neoplasias having mutations and/or altered expression of the core components of their Hippo pathway (e.g., MST1/2, LATS1/2, YAP and TAZ) may promote the migration, invasion, malignancy of cancer cells in a Hippo pathway associated neoplasia.

By “agent” is meant a peptide, nucleic acid molecule, or small compound.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

Typically, the compounds described herein, particularly in the context of drug screening, are small molecule chemical compound. It will be understood that the description of compounds herein is limited by principles of chemical bonding known to those skilled in the art. Accordingly, where a group may be substituted by one or more of a number of substituents, such substitutions are selected so as to comply with principles of chemical bonding with regard to valencies, etc., and to give compounds which are not inherently unstable.

Typically, alkyl groups described herein refer to a branched or straight-chain monovalent saturated aliphatic hydrocarbon radical of, for example, 1-30 carbon atoms (e.g., 1-16 carbon atoms, 6-20 carbon atoms, 8-16 carbon atoms, or 4-18 carbon atoms, 4-12 carbon atoms, etc.). In some embodiments, the alkyl group may be substituted with 1, 2, 3, or 4 substituent groups as defined herein. Alkyl groups may have from 1-26 carbon atoms. In other embodiments, alkyl groups will have from 6-18 or from 1-8 or from 1-6 or from 1-4 or from 1-3 carbon atoms, including for example, embodiments having one, two, three, four, five, six, seven, eight, nine, or ten carbon atoms. Any alkyl group may be substituted or unsubstituted. Examples of alkyl groups include methyl, ethyl, propyl, butyl, pentyl, hexyl, heptyl, octyl, nonyl, decyl, undecyl, and dodecyl groups. Heteroalkyl groups may refer to branched or straight-chain monovalent saturated aliphatic hydrocarbon radicals with one or more heteroatoms (e.g., N, O, S, etc.) in the carbon chain. Heteroalkyl groups may have 1-30 carbon atoms (e.g., 1-16 carbon atoms, 6-20 carbon atoms, 8-16 carbon atoms, or 4-18 carbon atoms, 4-12 carbon atoms, etc.). In some embodiments, the heteroalkyl group may be substituted with 1, 2, 3, or 4 substituent groups as defined herein. Heteroalkyl groups may have from 1-26 carbon atoms. In other embodiments, heteroalkyl groups will have from 6-18 or from 1-8 or from 1-6 or from 1-4 or from 1-3 carbon atoms, including for example, embodiments having one, two, three, four, five, six, seven, eight, nine, or ten carbon atoms. In some embodiments, the heteroalkyl group can be further substituted with 1, 2, 3, or 4 substituent groups as described herein for alkyl groups. Examples of heteroalkyl groups are an alkoxy. Alkoxy substituent groups or alkoxy-containing substituent groups may be substituted by, for example, one or more alkyl groups.

Aryl groups may be aromatic mono- or polycyclic radicals of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl. Typically, heteroaryls include mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group. Examples of heteroaryl groups are pyridyl, benzooxazolyl, benzoimidazolyl, and benzothiazolyl.

The term “substituent” refers to a group “substituted” on, e.g., an alkyl, at any atom of that group, replacing one or more hydrogen atoms therein (e.g., the point of substitution). In some aspects, the substituent(s) on a group are independently any one single, or any combination of two or more of the permissible atoms or groups of atoms delineated for that substituent. In another aspect, a substituent may itself be substituted with any one of the substituents described herein. Substituents may be located pendant to the hydrocarbon chain.

A substituted hydrocarbon group may have as a substituent one or more hydrocarbon radicals, substituted hydrocarbon radicals, or may comprise one or more heteroatoms. Examples of substituted hydrocarbon radicals include, without limitation, heterocycles, such as heteroaryls. Unless otherwise specified, a hydrocarbon substituted with one or more heteroatoms will comprise from 1-20 heteroatoms. In other embodiments, a hydrocarbon substituted with one or more heteroatoms will comprise from 1-12 or from 1-8 or from 1-6 or from 1-4 or from 1-3 or from 1-2 heteroatoms. Examples of heteroatoms include, but are not limited to, oxygen, nitrogen, sulfur, phosphorous, halogen (e.g., F, Cl, Br, I, etc.), boron, silicon, etc. In some embodiments, heteroatoms will be selected from the group consisting of oxygen, nitrogen, sulfur, phosphorous, and halogen (e.g., F, Cl, Br, I, etc.). In some embodiments, a heteroatom or group may substitute a carbon. In some embodiments, a heteroatom or group may substitute a hydrogen. In some embodiments, a substituted hydrocarbon may comprise one or more heteroatoms in the backbone or chain of the molecule (e.g., interposed between two carbon atoms, as in “oxa”). In some embodiments, a substituted hydrocarbon may comprise one or more heteroatoms pendant from the backbone or chain of the molecule (e.g., covalently bound to a carbon atom in the chain or backbone, as in “oxo”).

In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Unless otherwise noted, all groups described herein (e.g., alkyl, cycloalkyl, heteroalkyl, heterocycloalkyl, aryl, heteroaryl, alkylene, heteroalkylene, cylcoalkylene, heterocycloalkylene, etc.) may optionally contain one or more common substituents, to the extent permitted by valency. Common substituents include halogen (e.g., F, Cl, etc.), C₁₋₁₂ straight chain or branched chain alkyl, C₂₋₁₂ alkenyl, C₂₋₁₂ alkynyl, C₃-12 cycloalkyl, C₆-12 aryl, C₃-12 heteroaryl, C₃-12 heterocyclyl, C₁₋₁₂ alkylsulfonyl, nitro, cyano, —COOR, —C(O)NRR′, —OR, —SR, —NRR′, and oxo, such as mono- or di- or ti-substitutions with moieties such as halogen, fluoroalkyl, perfluoroalkyl, perfluroalkoxy, trifluoromethoxy, chlorine, bromine, fluorine, methyl, methoxy, pyridyl, furyl, triazyl, piperazinyl, pyrazoyl, imidazoyl, and the like, each optionally containing one or more heteroatoms such as halo, N, O, S, and P. R and R′ are independently hydrogen, C₁₋₁₂ alkyl, C₁₋₁₂haloalkyl, C₂₋₁₂ alkenyl, C₂₋₁₂ alkynyl, C₃₋₁₂ cycloalkyl, C₄₋₂₄ cycloalkylalkyl, C₆₋₁₂ aryl, C₇₋₂₄ aralkyl, C₃₋₁₂ heterocyclyl, C₃₋₂₄ heterocyclylalkyl, C₃₋₁₂ heteroaryl, or C₄₋₂₄ heteroarylalkyl. Further, as used herein, the phrase optionally substituted indicates the designated hydrocarbon group may be unsubstituted (e.g., substituted with H) or substituted. Typically, substituted hydrocarbons are hydrocarbons with a hydrogen atom removed and replaced by a substituent (e.g., a common substituent).

It is understood by one of ordinary skill in the chemistry art that substitution at a given atom is limited by valency. The use of a substituent (radical) prefix names such as alkyl without the modifier optionally substituted or substituted is understood to mean that the particular substituent is unsubstituted. However, the use of haloalkyl without the modifier optionally substituted or substituted is still understood to mean an alkyl group, in which at least one hydrogen atom is replaced by halo. Where a group may be substituted by one or more of a number of substituents, such substitutions are selected so as to comply with principles of chemical bonding with regard to valencies, etc., and to give compounds which are not inherently unstable. For example, any carbon atom will be bonded to two, three, or four other atoms, consistent with the four valence electrons of carbon. Additionally, when a structure has less than the required number of functional groups indicated, those carbon atoms without an indicated functional group are bonded to the requisite number of hydrogen atoms to satisfy the valency of that carbon.

As used herein, the term “isomers” are different compounds that have the same molecular formula. “Stereoisomers” are isomers that differ only in the way the atoms are arranged in space. As used herein, the term “isomer” includes any and all geometric isomers and stereoisomers. For example, “isomers” include geometric double bond cis- and trans-isomers, also termed E- and Z-isomers; R- and S-enantiomers; diastereomers, (d)-isomers and (l)-isomers, racemic mixtures thereof; and other mixtures thereof, as falling within the scope of this disclosure.

Geometric isomers can be represented by the symbol [symbol] which denotes a bond that can be a single, double or triple bond as described herein. Provided herein are various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

“Enantiomers” are a pair of stereoisomers that are non-superimposable mirror images of each other. A mixture of a pair of enantiomers in any proportion can be known as a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is an enantiomer, the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry at each asymmetric atom, as (R)- or (S)-. The present chemical entities, pharmaceutical compositions and methods are meant to include all such possible isomers, including racemic mixtures, optically substantially pure forms and intermediate mixtures. Optically active (R)- and (S)-isomers can be prepared, for example, using chiral synthons or chiral reagents, or resolved using conventional techniques.

The “enantiomeric excess” (ee) or “% enantiomeric excess” of a composition can be calculated using the equation shown below. In the example shown below, a composition contains 90% of one enantiomer, e.g., the S enantiomer, and 10% of the other enantiomer, e.g., the R enantiomer. ee=(90-10)/100=80%. Thus, a composition containing 90% of one enantiomer and 10% of the other enantiomer is said to have an enantiomeric excess of 80%. Some compositions described herein contain an enantiomeric excess of at least about 50%, about 75%, about 90%, about 95%, or about 99% of the S enantiomer. In other words, the compositions contain an enantiomeric excess of the S enantiomer over the R enantiomer. In other embodiments, some compositions described herein contain an enantiomeric excess of at least about 50%, about 75%, about 90%, about 95%, or about 99% of the R enantiomer. In other words, the compositions contain an enantiomeric excess of the R enantiomer over the S enantiomer.

For instance, an isomer/enantiomer can, in some embodiments, be provided substantially free of the corresponding enantiomer, and can also be referred to as “optically enriched,” “enantiomerically enriched,” “enantiomerically pure” and “non-racemic,” as used interchangeably herein. These terms refer to compositions in which the percent by weight of one enantiomer is greater than the amount of that one enantiomer in a control mixture of the racemic composition (e.g., greater than 1:1 by weight). For example, an enantiomerically enriched preparation of the S enantiomer means a preparation of the compound having greater than about 50% by weight of the S enantiomer relative to the R enantiomer, such as at least about 75% by weight, further such as at least about 80% by weight. In some embodiments, the enrichment can be much greater than about 80% by weight, providing a “substantially enantiomerically enriched,” “substantially enantiomerically pure” or a “substantially non-racemic” preparation, which refers to preparations of compositions which have at least about 85% by weight of one enantiomer relative to other enantiomer, such as at least about 90% by weight, and further such as at least about 95% by weight. In certain embodiments, the compound provided herein is made up of at least about 90% by weight of one enantiomer. In other embodiments, the compound is made up of at least about 95%, about 98%, or about 99% by weight of one enantiomer.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, the compound mixture has an (S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more. In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

In other embodiments, the compound mixture contains identical chemical entities except for their stereochemical orientations, namely (S)- or (R)-isomers. For example, if a compound disclosed herein has a —CH(R)- unit, and R is not hydrogen, then the —CH(R)— is in an (S)- or (R)-stereochemical orientation for each of the identical chemical entities. In some embodiments, the mixture of identical chemical entities is a racemic mixture of (S)- and (R)-isomers. In another embodiment, the mixture of the identical chemical entities (except for their stereochemical orientations), contain predominately (S)-isomers or predominately (R)-isomers. For example, the (S)-isomers in the mixture of identical chemical entities are present at about 55%, about 60%, about 65%, about 70%, about 75%, about 80%, about 85%, about 90%, about 95%, about 96%, about 97%, about 98%, about 99%, about 99.5%, or more, relative to the (R)-isomers. In some embodiments, the (S)-isomers in the mixture of identical chemical entities are present at an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

In another embodiment, the (R)-isomers in the mixture of identical chemical entities (except for their stereochemical orientations), are present at about 55%, about 60%, about 65%, about 70%, about 75%, about 80%, about 85%, about 90%, about 95%, about 96%, about 97%, about 98%, about 99%, about 99.5%, or more, relative to the (S)-isomers. In some embodiments, the (R)-isomers in the mixture of identical chemical entities (except for their stereochemical orientations), are present at a (R)-enantiomeric excess greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

Enantiomers can be isolated from racemic mixtures by any method known to those skilled in the art, including chiral high-pressure liquid chromatography (HPLC), the formation and crystallization of chiral salts, or prepared by asymmetric syntheses. See, for example, Enantiomers, Racemates and Resolutions (Jacques, Ed., Wiley Interscience, New York, 1981); Wilen et al., Tetrahedron 33:2725 (1977); Stereochemistry of Carbon Compounds (E. L. Eliel, Ed., McGraw-Hill, N Y, 1962); and Tables of Resolving Agents and Optical Resolutions p. 268 (E. L. ElM, Ed., Univ. of Notre Dame Press, Notre Dame, Ind. 1972).

Optical isomers can be obtained by resolution of the racemic mixtures according to conventional processes, e.g., by formation of diastereoisomeric salts, by treatment with an optically active acid or base. Examples of appropriate acids are tartaric, diacetyltartaric, dibenzoyltartaric, ditoluoyltartaric, and camphorsulfonic acid. The separation of the mixture of diastereoisomers by crystallization followed by liberation of the optically active bases from these salts affords separation of the isomers. Another method involves synthesis of covalent diastereoisomeric molecules by reacting disclosed compounds with an optically pure acid in an activated form or an optically pure isocyanate. The synthesized diastereoisomers can be separated by conventional means such as chromatography, distillation, crystallization or sublimation, and then hydrolyzed to deliver the enantiomerically enriched compound. Optically active compounds can also be obtained by using active starting materials. In some embodiments, these isomers can be in the form of a free acid, a free base, an ester or a salt.

In certain embodiments, the pharmaceutically acceptable form is a tautomer. As used herein, the term “tautomer” is a type of isomer that includes two or more interconvertible compounds resulting from at least one formal migration of a hydrogen atom and at least one change in valency (e.g., a single bond to a double bond, a triple bond to a single bond, or vice versa). “Tautomerization” includes prototropic or proton-shift tautomerization, which is considered a subset of acid-base chemistry. “Prototropic tautomerization” or “proton-shift tautomerization” involves the migration of a proton accompanied by changes in bond order. The exact ratio of the tautomers depends on several factors, including temperature, solvent, and pH. Where tautomerization is possible (e.g., in solution), a chemical equilibrium of tautomers can be reached. Tautomerizations (i.e., the reaction providing a tautomeric pair) can be catalyzed by acid or base or can occur without the action or presence of an external agent. Exemplary tautomerizations include, but are not limited to, keto-to-enol; amide-to-imide; lactam-to-lactim; enamine-to-imine; and enamine-to-(a different) enamine tautomerizations. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, 4C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, substitution with heavier isotopes such as deuterium affords greater stability (for example, increased half-life or reduced loading requirements). Isotopically-labeled compounds are prepared by any suitable method or by processes using an appropriate isotopically-labeled reagent in place of the non-labeled reagent otherwise employed.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “computer modeling” is meant the application of a computational program to determine one or more of the following: the location and binding proximity of a ligand to a binding moiety, the occupied space of a bound ligand, the amount of complementary contact surface between a binding moiety and a ligand, the deformation energy of binding of a given ligand to a binding moiety, and some estimate of hydrogen bonding strength, van der Waals interaction, hydrophobic interaction, and/or electrostatic interaction energies between ligand and binding moiety. Computer modeling can also provide comparisons between the features of a model system and a candidate compound. For example, a computer modeling experiment can compare a pharmacophore model of the invention with a candidate compound to assess the fit of the candidate compound with the model.

By a “computer system” is meant the hardware means, software means and data storage means used to analyze atomic coordinate data. The minimum hardware means of the computer-based systems of the present invention comprises a central processing unit (CPU), input means, output means and data storage means. Desirably a monitor is provided to visualize structure data. The data storage means may be RAM or means for accessing computer readable media of the invention. Examples of such systems are microcomputer workstations available from Silicon Graphics Incorporated and Sun Microsystems running Unix based, Windows NT or IBM OS/2 operating systems.

By “computer readable media” is meant any media which can be read and accessed directly by a computer e.g. so that the media is suitable for use in the above-mentioned computer system. The media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include proliferative diseases such as cancer (e.g., sarcoma, pancreas, prostate, head and neck, liver, and breast cancer).

The term “proliferative disease,” as used herein, is a disease that is caused by or results in inappropriately high levels of cell division, inappropriately low levels of apoptosis, or both. Cancer is an example of a proliferative disease. Examples of cancer include, but are not limited to, (e.g., sarcoma, pancreas, prostate, head and neck, liver, and breast cancer). Other examples of such cancers include lung cancer, e.g., lung adenocarcinoma, small cell lung cancer, non-small cell lung cancer (“NSCLC”), squamous cell cancer (e.g., epithelial squamous cell cancer), vulvar cancer, thyroid cancer, and squamous carcinoma of the lung, biliary tract malignancies, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, as well as head and neck cancer. In yet other embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitt's Lymphoma, B-cell ALL, Philadelphia chromosome positive ALL, Philadelphia chromosome positive CML, lymphoma, leukemia, multiple myeloma, myeloproliferative diseases, large B cell lymphoma, and B cell Lymphoma.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of an agent described herein used to decrease or stabilize cell proliferation, cell survival, or tumor mass.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

The term “pharmaceutical composition,” as used herein, represents a composition containing a compound described herein formulated with a pharmaceutically acceptable excipient or carrier. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

Useful pharmaceutical carriers for the preparation of the compositions hereof, can be solids, liquids, or gases. Thus, the compositions can take the form of tablets, pills, capsules, suppositories, powders, enterically coated or other protected formulations (e.g., binding on ion-exchange resins or packaging in lipid-protein vesicles), sustained release formulations, solutions, suspensions, elixirs, and aerosols. The carrier can be selected from the various oils including those of petroleum, animal, vegetable or synthetic origin, e.g., peanut oil, soybean oil, mineral oil, and sesame oil. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, and ethanol. The compositions may be subjected to conventional pharmaceutical additives such as preservatives, stabilizing agents, wetting or emulsifying agents, salts for adjusting osmotic pressure, and buffers. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for administration to the recipient.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

As used herein, the term “pharmaceutically acceptable salt” refers to salts of any of the compounds described herein that within the scope of sound medical judgment, are suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, dichloroacetate, digluconate, dodecylsulfate, ethanesulfonate, formate, fumarate, glucoheptonate, glutamate, glycerophosphate, hemisulfate, heptonate, hexanoate, hippurate, hydrobromide, hydrochloride, hydroiodide, 2-hydroxy-ethanesulfonate, isethionate, lactobionate, lactate, laurate, lauryl sulfate, malate, maleate, malonate, mandelate, methanesulfonate, mucate, 2-naphthalenesulfonate, nicotinate, nitrate, oleate, oxalate, palmitate, pamoate, pantothenate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative basic salts include alkali or alkaline earth metal salts include sodium, lithium, potassium, calcium, and magnesium, aluminum salts, as well as nontoxic ammonium, quaternary ammonium, and amine cations, including, but not limited to ammonium, tetramethylammonium, tetraethylammonium, methylamine, dimethylamine, trimethylamine, triethylamine, caffeine, and ethylamine.

Pharmaceutically acceptable salts may be prepared from a compound of the present disclosure having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)-amine, 2-hydroxy-tert-butylamine, or tris-(hydroxymethyl)methylamine, N, N,-di-lower alkyl-N-(hydroxy lower alkyl)-amines, such as N,N-dimethyl-N-(2-hydroxyethyl)-amine, or tri-(2-hydroxyethyl)amine; N-methyl-D-glucamine; and amino acids such as arginine, lysine, and the like.

Pharmaceutically acceptable salts may be prepared from a compound of the present disclosure having a basic functional group, such as an amino functional group, and a pharmaceutically acceptable inorganic or organic acid. Suitable acids include, but are not limited to, hydrogen sulfate, citric acid, acetic acid, oxalic acid, hydrochloric acid, hydrogen bromide, hydrogen iodide, nitric acid, phosphoric acid, isonicotinic acid, lactic acid, salicylic acid, tartaric acid, ascorbic acid, succinic acid, maleic acid, besylic acid, fumaric acid, gluconic acid, glucaronic acid, saccharic acid, formic acid, benzoic acid, glutamic acid, methanesulfonic acid, ethanesulfonic acid, benzenesulfonic acid, and p-toluenesulfonic acid.

By “reduces” is meant a negative alteration of, for example, at least 10%, 25%, 50%, 75%, or 100%.

By “reference” is meant a standard or control condition. In one embodiment, a reference is the condition of an untreated subject or corresponding cell type. In another embodiment, a reference is the condition of a treated subject or cell type prior to treatment.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/mL denatured salmon sperm DNA (ssDNA). In a most preferred embodiment, hybridization will occur at 42° C. in 250 mM NaCl, 25 mM trisodium citrate, 1% SDS, 50% formamide, and 200 μg/mL ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.10% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 (1A-1B) provides images of morphological profiling by Cell Painting. FIG. 1A displays example Cell Painting images from each of the five channels for a negative control sample (no gene introduced). FIG. 1B, from left to right, shows cell and nucleus outlines found by segmentation in CellProfiler; raw profiles (2769 dimensional) containing median and median absolute deviation of each of 1384 measurements over all the cells in a sample, plus cell count; processed profiles which are made less redundant by feature selection and Principal Component Analysis; dendrogram constructed based on the processed profiles. Replicates are merged to produce a profile for each gene which is then compared against others in the experiment to look for similarities and differences.

FIG. 2 is a characterization for matching the morphological signature of a gene query to the signature(s) of compounds in a library. Connections which show both positive and negative large correlations are considered as matches. In order to make signatures comparable across datasets, all the features are normalized according to the negative controls in the experiment they associate with.

FIG. 3 is a graph quantifying viable mouse sarcoma cell number vs. time for treatment with 1 μM N-benzylquinazolin-4-amine (NB4A)(upper curve) and DMSO (lower curve) control for three independent experiments with three replicates per experiment.

FIG. 4 provides two graphs showing Yap1 expression in sarcoma cells treated with N-benzylquinazolin-4-amine (NB4A) (left panel) and Yap1 target Foxm1 expression in sarcoma cells treated with N-benzylquinazolin-4-amine (NB4A) (right panel), where the expression of Yap1 and Foxm1 is normalized to housekeeping genes Hprt, Sdha, orHprt and Sdha combined. This experiment shows that Yap1 gene expression is not affected by NB4A treatment, but that Foxm1 expression is reduced.

FIG. 5 provides two graphs showing Ccl2 expression in sarcoma cells treated with N-benzylquinazolin-4-amine (NB4A) (left panel) and Hbegf expression in sarcoma cells treated with N-benzylquinazolin-4-amine (NB4A) (right panel), where the expression of Ccl2 and Hbegf is normalized to housekeeping genes Hprt, Sdha, or Hprt and Sdha combined. This experiment shows that Ccl2 expression is significantly decreased by treatment with NB4A.

FIG. 6 provides two graphs showing the expression of the Yap1 target Birc5 (left) in sarcoma cells treated with N-benzylquinazolin-4-amine (NB4A) (left panel) and Rela, a p65 subunit, expression in sarcoma cells treated with N-benzylquinazolin-4-amine (NB4A) (right panel), where the expression of Birc5 and Rela is normalized to housekeeping genes Hprt, Sdha, or Hprt and Sdha combined.

FIG. 7 is a set of four bar graphs showing normalized expression of Yap1, Foxm1, Birc5, Rela, Ccl2, or Hbegf in sarcoma cells treated with N-benzylquinazolin-4-amine or a DMSO control for 48 hours. The expression of Yap1, Foxm1, Birc5, Rela, Ccl2, or Hbegf was normalized to housekeeping genes Hprt, Sdha, or Hprt and Sdha combined.

FIG. 8 is a set of four bar graphs showing normalized expression of Yap1, Foxm1, Birc5, Rela, Ccl2, or Hbegf in sarcoma cells treated with N-benzylquinazolin-4-amine or a DMSO control for 72 hours. The expression of Yap1, Foxm1, Birc5, Rela, Ccl2, or Hbegf was normalized to housekeeping genes Hprt, Sdha, or Hprt and Sdha combined.

FIG. 9 is a graph of referent gene comparison displayed as cycle threshold (CT) vs. time. The genes are from left to right Hprt, Sdha, and Hprt and Sdha combined, at 48 h and 72 h.

FIG. 10 illustrates that cell painting profiles identify compounds impacting the p38a pathway. Compounds predicted to perturb p38 activity (triangles) and a set of neutral compounds (Cell Painting profile correlation values to p38a between −0.2 to 0.2; circles) were tested for their influence on p38a activity at 1 (mu)M concentration using a two sided t-test on the single cell distributions of a p38 activity reporter as described in Kaufman, Tom, et al. “Visual Barcodes for Multiplexing Live Microscopy-Based Assays.” (2020), hereby incorporated by reference in its entirety and particularly in relation to the p38 reporter (FDR-adjusted −log₁₀ p-values shown. Two potential inhibitors were found (BRD-K38197229, 3-(butylamino)-4-phenoxy-5-sulfamoylbenzoic acid, referred to in the figure as <K381>) and BRD-A64933752, 2-(6-amino-2-anilinopurin-9-yl)-5-(hydroxymethyl)oxolane-3,4-diol, referred to in the figure as <A649>); an additional compound (BRD-K52394958, 5-Fluoro-3-[2-[4-methoxy-4-[[(R)-phenylsulfinyl]methyl]piperidin-1-yl]ethyl]-1H-indole, referred to in the figure as <523> was also identified via an alternative statistical test as shown in FIG. 13. BRD-54330070 (4-(4-Fluorophenyl)-2-(4-hydroxyphenyl)-5-(4-pyridyl)imidazole), SB202190), referred to in the figure as K543 is a known p38 inhibitor also identified as a match.

FIG. 11 shows that the predicted compounds impact p38 activity in a single-cell reporter assay. Panel a provides the same experiment as shown in FIG. 10, with the exception of using a Kolmogorov-Smirnoff (KS) analysis to detect differences in distribution instead of shifts in the mean. An additional hit, K523 (BRD-K52394958, 5-Fluoro-3-[2-[4-methoxy-4-[[(R)-phenylsulfinyl]methyl]piperidin-1-yl]ethyl]-1H-indole) was identified with this analysis. Panels b-i illustrate the single cell distribution plots showing the shifts induced, at both 1 (mu)M and 10 (mu)M, by SB202190, a known p38 inhibitor (panels b-c), by the hits identified by the t-test analysis shown in FIG. 10 (panels d-g), and by the hit from the KS analysis (panels (h-i).

FIG. 12 (12A-12B) show that Cell Painting profiles identify compounds impacting PPARGC1A (PGC-1α) overexpression phenotypes. FIG. 12A is Cell Painting images for PPARGC1A (PGC-1α) overexpression compared to negative control (EMPTY). Scale bar=60 μm; images are cropped roughly 30% relative to the field of view. FIG. 12B provides the correlation of compounds to PGC-1α overexpression that were chosen for further study (hence all samples are below ˜-0.35 or above ˜0.35 on the X axis). The samples with high correlation show generally high blobbiness, as plotted on the Y axis as number of standard deviations (normalized to the negative controls).

FIG. 13 (13A-13B) illustrate that certain subpopulations of cells are over- or under-represented when PPARGC1A is overexpressed. Following the procedure described in Rohban et al., eLife 2017; 6:e24060, incorporated herein by reference in its entirety, cells were clustered based on their morphological profiles and subpopulations were identified which were (FIG. 13A) over- or (FIG. 13B) under-represented when PPARGC1A is overexpressed. Scale bars=39.36 μM.

FIG. 14 (14A-14B) provides the PPARG reporter gene assay dose-response curves in the absence (FIG. 14A) or presence (FIG. 14B) of added PPARG agonist, Rosiglitazone. Representative data of the ten most active compounds is shown and reported as normalized light units. Compounds BRD-K95785537 and BRD-K29542628 are structurally related pyrazolo-pyrimidines.

FIG. 15 (15A-15B) shows compounds predicted to influence pathways containing PGC-1a impact an ERRa reporter assay in 293T cells. In this reporter system, a mammalian one-hybrid fusion protein containing the Gal4 DNA binding domain and the ERR alpha ligand binding domain is co-expressed with the Firefly luciferase gene under control of the Gal4 Upstream Activating Sequence. Renilla luciferase was included for normalization. The assay was performed in the presence (a) or absence (b) of ectopically expressed PGC-Ta; their behavior being similar in these two conditions suggests, but does not prove, that the compounds do not directly target PGC-1a but instead modulate other targets in the relevant pathway, consistent with having been discovered by the morphological matching approach which assesses impact on the cell system rather than a particular desired target.

FIG. 16 shows each indicated compound impact a mitochondrial motility assay in rat cortical neurons. 23 compounds were tested because one of the original 24 tested in FIG. 3c became unavailable. (Panel a) For most compounds, the integrated distance traveled for each motile mitochondrion (the length of travel, or the sum of all movements, including changes in direction) is comparable to the negative control (Mock), but a few compounds (A01, A06, A10, A11, B03, and B04) consistently have a z-score >3, as does the positive control, Calcimycin, a calcium ionophore that arrests mitochondria. Two separate experiments are plotted (week 1 and week 2), and the values are the Z-prime factor of the Kolmogorov-Smimov (KS) statistic calculated for each compound. Panel b provides the mean values of the mitochondrial distance; these are the values that underlie the statistical analysis in FIG. 16A. Panel c provides the average intensity of tetramethylrhodamine ethyl ester (TMRE) staining which reflects the mitochondrial membrane potential, a measure of mitochondrial function. Interestingly, A01, A06 and A11 all show normal levels of TMRE staining, suggesting a specific effect on mitochondrial motility rather than a more general decrease in neuronal or mitochondrial health. This cannot be said for B03 and B04 (and A10 to a lesser extent), which apparently reduce membrane potential, although additional validation with TMRE is needed to conclude that they are in fact detrimental to cell health. Of note, four of these compounds were also active in the PPARG reporter assay (FIGS. 14A and 14B): A01 and A11 are structurally related molecules of the pyrazolo-pyrimidine family, 1-Naphthyl-PP1 and PP2, which are Src family kinase inhibitors with additional targets including TGFbeta receptors and others. A06 is Phorbol myristate acetate (also referred to as TPA, PMA). B09 is annotated as an HSP-90 inhibitor CCT-018159.

FIG. 17 provides Cell Painting results illustrating that YAP1 in U2OS produces elongated cells with more cell protrusions, lower RNA staining, and disjoint, bright mitochondria patterns.

FIG. 18 (18A-18I) illustrates the use of Cell Painting profiles to identify compounds impacting the Hippo pathway. FIG. 18A provides the Relative transcript levels of YAP1, CTGF, and CYR61 in H9 human pluripotent stem cells treated with NB4A or DMSO control for 24 hrs. *P<0.05; **P<0.01; ***P<0.001; ****P<0.0001 (one-way ANOVA with Dunnett's multiple comparisons test). Mean 1 SEM. n=3. FIG. 18B provides representative images of YAP1 immunofluorescence (left, labelled DAPI) and quantification of mean nuclear/cytoplasmic YAP1 intensity (right, Labelled YAP1) in H9 cells after treatment with 10 μM NB4A or DMSO control for 24 hours. The integrated signal intensities for the measurements are provided on the right, with a p=value as determined by t-tailed student's t-test. n=3; 3 fields per condition per experiment. FIG. 18C shows the western blot analysis of phospho-YAP1 (S127) and total YAP1 from H9 cells treated with DMSO or NB4A for 24 hrs. Latrunculin-A (Lat. A), an actin inhibitor, is used as a positive control for YAP1 phosphorylation. FIG. 18D show the normalized enrichment scores of GSEA Hallmark pathways up- and down-regulated in NB4A-treated vs. control KP230 cells. Up to the 10 most significant pathways in each comparison are shown (FDR-adjusted P<0.25). n=3. FIG. 18E provides representative western blot demonstrating Yap1 expression in NB4A-treated and control KP230 cells. FIG. 18F shows the immunofluorescence-based analysis of total Yap1 expression in NB4A-treated and control KP230 cells with relative p-values as determined by two-tailed student's t-test. Barrs show the mean±SEM. n=3. FIG. 18G shows the immunofluorescence-based analysis of nuclear Yap1 expression in NB4A-treated and control KP230 cells (normalized to total Yap1). Two-tailed student's t-test. Mean±SEM. n=3. For FIGS. 18F and 18G, the “normalized integrated density” of an image is equivalent to integrated density normalized to cell number. FIG. 18H provides representative images for the analyses shown in FIGS. 18D and 18E. 5 fields per condition per experiment were acquired. Scale bar=100 μM. FIG. 18I shows the growth curves of NB4A-treated and control KP230 (left) and TC32 (right) sarcoma cells. **P<0.01; ****P<0.0001 DMSO vs. NB4A (72 hrs.; 2-way ANOVA with Sidak's multiple comparisons test). Mean 1 SEM. n=3. For FIGS. 18D and 18I, cells were treated with 10 μM NB4A daily for 72 hours.

FIG. 19 illustrates that some compounds do not dampen expression from a YAP1-responsive reporter. A TEAD luciferase reporter was co-transfected with or without a Yap expression construct into HEK293T cells followed by treatment for 48 hours with DMSO or the indicated compounds. The data shown are the average of three samples within a representative experiment.

FIG. 20 (20A-20D) provides a table illustrating the RNA-sequencing-based enriched analysis of Hallmark gene sets up- and down-regulated in KP230 cells by NB4A, and some relevant parameters in that analysis provided in FIG. 18D. FIG. 20C identifies the Hallmark genes upregulated by NB4A treatment. FIG. 20D identifies the Hallmark genes downregulated by NB4A treatment.

FIG. 21 (21A-21G) provides that the predicted Hippo pathway-modulating compounds impact proliferation in a cell type-specific manner. FIGS. 21A and 21B: Growth curves of YAP1-dependent human sarcoma cells treated with 10 μuM NB4A or DMSO control. FIG. 21C: Growth curve of HCT-116 colon cancer cells treated with 10 μuM NB4A or DMSO control. FIGS. 21A-C are not significantly different at any time point (2-way ANOVA with Sidak's multiple comparisons test). n=3. Mean 1 SEM. FIG. 21D: Growth curve of HCT-116 cells infected with YAP1-targeting shRNAs or scrambled shRNA control (sh:SCR); no conditions were significantly different at any time point (vs. sh: SCR; 2-way ANOVA with Dunnett's multiple comparisons test). n=3. Mean 1 SEM. FIG. 21E: Relative YAP1 expression in the cells depicted in panel d ****P<0.0001 vs. sh:SCR (1-way ANOVA with Dunnett's multiple comparisons test). FIG. 21F: Growth curves of KP230 cells treated with 10 μM BRD-K28862419, BRD-K34692511, or DMSO control. **P<0.01 vs. DMSO (72 hrs.; 2-way ANOVA with Dunnett's multiple comparisons test). n=2 Mean±SEM. FIG. 21G: Percent viability of KP230 cells depicted in panel f **P<0.01 vs. DMSO (72 hrs.; 2-way ANOVA with Dunnett's multiple comparisons test). n=3. Mean 1 SEM. For panels a, b, c, f, and g, cells were treated with 10 μuM of the indicated inhibitor daily for 72 hours.

FIG. 22 (22A-22D) illustrates that BRD-K34692511 upregulates YAP1- and target-gene mRNA levels in murine periosteal cells. FIGS. 22A and 22B: YAP1 and Cyr61 mRNA levels in murine periosteal cells after 48 hours of treatment with BRD-K34692511 (K34) in the presence or absence of doxycycline-induced YAP^{S127A}. FIGS. 22C and 22D: YAP1 and Cyr61 mRNA levels after 1 and 4 hours of treatment. Gene expression was evaluated by one and two-way ANOVA with Tukey post hoc test n=3/group/time-point. * indicates p<0.05 compared to untreated controls.

FIG. 23 shows that BRD-K28862419 and BRD-K34692511 did not dramatically impact mRNA levels of hippo pathway members in hPSCs. Relative transcript levels of YAP1, CTGF, and CYR61 from H9 hPSCs treated with DMSO, BRD-K28862419, or BRD-K34692511 for 24 hrs. Error bars represent mean+SEM, from n=3 biological replicates (one-way ANOVA with Dunnett multiple comparison test).

### DETAILED DESCRIPTION OF THE INVENTION

The invention features compositions and methods that inhibit the proliferation, survival, or growth of NF-κB and/or Hippo associated neoplasias. Without wishing to be bound by theory, the NF-κB and/or Hippo signaling pathways may function independently in neoplasias. However, these pathways may also function and interact with each other such that the compounds identified herein may modulate signaling in one or both of the NF-κB or Hippo signaling pathway in cells.

The invention is based, at least in part, on the discovery of agents (e.g., N-benzylquinazolin-4-amine) that modulate the NF-κB growth pathway. The agents were identified using a two-pronged approach that combined a Cell Painting morphological analysis with transcriptional data analysis. In particular, overexpression of YAP1 and TAZ in U2OS cells yielded a distinctive morphology (via the Cell Painting set of organelle stains) and overexpression of NF-κB members gave the opposite visual phenotype, implying a negative regulation of YAP1 and TAZ. Here, it was discovered that a computer-based analysis of images of cells treated with candidate compounds identified thirty compounds that appeared to phenocopy YAP1/TAZ negative or positive regulation. The compounds were then characterized for function as described herein. Taken together, these studies revealed an unexpected relationship in human cells between two major signaling pathways, Hippo and NF-κB. Both pathways are under intense study for their involvement in cancer. It has also been discovered that YAP1/TAZ-directed transcription is negatively regulated by NF-κB pathway effectors. Without intending to be bound by theory, the data suggests a novel regulatory mechanism that is independent of upstream Hippo kinases.

To date, there has been little evidence of the intersection between these important signaling pathways. Recent work examining osteoclast-osteoblast differentiation has suggested that Hippo pathway kinases, such as Mst2, also known as Human serine/threonine-protein kinase 3 (STK3), may affect the NF-κB pathway through phosphorylation of IkB proteins, thereby promoting nuclear translocation of NF-κB transcription factors. TAZ was found to be a direct target of NF-κB transcription factors and its expression is regulated via NF-κB signaling. Recent work, however, supports a possible additional mode of interaction, whereby regulators of NF-κB signaling directly regulate the function of YAP1 and TAZ as transcriptional co-factors. Recent work has demonstrated, in Drosophila, that NF-κB activation via Toll receptor signaling negatively regulates the transcriptional activity of Yorkie, the homolog of YAP1/TAZ, through activation of canonical hippo pathway kinases. Recent work identifies, for the first time in a mammalian system, that a negative regulatory relationship exists between NF-κB activation and YAP1/TAZ transcriptional function. Furthermore, this regulation of YAP1/TAZ occurs in a manner that is independent of Hippo pathway-mediated phosphorylation events on YAP1/TAZ, suggesting a more direct relationship between NF-κB and YAP1/TAZ signaling.

Compounds that Modulate Proliferation

Accordingly, the invention provides compounds useful for treating NF-κB associated neoplasias (e.g., N-benzylquinazolin-4-amine, and derivatives or analogs thereof).

Other compounds useful for treating NF-κB associated neoplasias include N-[[(8R,9S)-6-[(2R)-1-hydroxypropan-2-yl]-8-methyl-5-oxo-10-oxa-1,6,13,14 tetrazabicyclo[10.2.1]pentadeca-12(15),13-dien-9-yl]methyl]-N-methyl-4 phenoxybenzenesulfonamide, (4S,5R)-5-((dimethylamino)methyl)-2-((R)-1-hydroxypropan-2-yl)-4-methyl-8-(pyridin-2-ylethynyl)-2,3,4,5-tetrahydrobenzo[b][1,4,5]oxathiazocine 1,1-dioxide, N-[(4S,7S,8S)-8-methoxy-4,7,10-trimethyl-11-oxo-2-oxa-5,10-diazabicyclo[10.4.0]hexadeca-1(12),13,15-trien-15-yl]-4-phenylbenzamide, 1-[(4R,7S,8S)-8-methoxy-4,7,10-trimethyl-11-oxo-2-oxa-5,10-diazabicyclo[10.4.0]hexadeca-1(12),13,15-trien-14-yl]-3-[4-(trifluoromethyl)phenyl]urea, 2-[(3S,6aR,8S,10aR)-3-hydroxy-1-(3-methoxyphenyl)sulfonyl-3,4,6,6a,8,9,10,10a-octahydro-2H-pyrano[2,3-c][1,5]oxazocin-8-yl]-1-(4-phenyl-1-piperazinyl)ethanone, 1-pyridin-4-yl-3-(2,4,6-trichlorophenyl)urea, 4-(5,7,7,10,10-pentamethyl-8,9-dihydronaphtho[2,3-b][1,4]benzodiazepin-13-yl)benzoic acid (LE-135), 1-(3,3a,4,5,6,6a-hexahydro-1H-cyclopenta[c]pyrrol-2-yl)-3-(4-methylphenyl)sulfonylurea, 5-(1,4-diazepan-1-ylsulfonyl)-2H-isoquinolin-1-one, 1-[(3,4-dimethoxyphenyl)methyl]-6,7-dimethoxyisoquinoline, 5-(4-chlorophenyl)-6-ethylpyrimidine-2,4-diamine, 7-hydroxy-3-(4-hydroxyphenyl)chromen-4-one, N-benzylquinazolin-4-amine, (2R,3R,3aS,9bS)-7-(1-cyclohexenyl)-N-(cyclopropylmethyl)-3-(hydroxymethyl)-6-oxo-1,2,3,3a,4,9b-hexahydropyrrolo[2,3-a]indolizine-2-carboxamide, N-[(1R,3R,4aS,9aR)-3-[2-[(3-fluorophenyl)methylamino]-2-oxoethyl]-1-(hydroxymethyl)-3,4,4a,9a-tetrahydro-TH-pyrano[3,4-b]benzofuran-6-yl]-1,3-benzodioxole-5-carboxamide, (1S,9R,10R,11R)-11-N-ethyl-10-(hydroxymethyl)-5-(2-methoxyphenyl)-6-oxo-12-N-propyl-7,12-diazatricyclo[7.2.1.02,7]dodeca-2,4-diene-11,12-dicarboxamide, N-[(1S,3S,4aR,9aS)-1-(hydroxymethyl)-3-[2-oxo-2-(1-piperidinyl)ethyl]-3,4,4a,9a-tetrahydro-1H-pyrano[3,4-b]benzofuran-6-yl]-4-oxanecarboxamide, N-[(5S,6S,9S)-8-(cyclopropylmethyl)-5-methoxy-3,6,9-trimethyl-2-oxo-11-oxa-3,8-diazabicyclo[10.4.0]hexadeca-1(12),13,15-trien-14-yl]-2-fluorobenzamide, N-[(4R,7S,8R)-8-methoxy-4,7,10-trimethyl-11-oxo-5-(1,3-thiazol-2-ylmethyl)-2-oxa-5,10-diazabicyclo[10.4.0]hexadeca-1(12),13,15-trien-14-yl]cyclohexanecarboxamide, 2-[(3S,6aR,8R,10aR)-1-(1,3-benzodioxol-5-ylmethyl)-3-hydroxy-3,4,6,6a,8,9,10,10a-octahydro-2H-pyrano[2,3-c][1,5]oxazocin-8-yl]-1-piperidin-1-ylethanone, 2-[(1R,3R,4aS,9aR)-1-(hydroxymethyl)-6-[(3-methoxyphenyl)sulfonylamino]-3,4,4a,9a-tetrahydro-1H-pyrano[3,4-b]benzofuran-3-yl]acetic acid methyl ester, 4-fluoro-N-[(2R,3R)-5-[(2R)-1-hydroxypropan-2-yl]-3-methyl-2-(methylaminomethyl)-6-oxo-3,4-dihydro-2H-1,5-benzoxazocin-10-yl]benzenesulfonamide, N-[(2S,3S,6R)-2-(hydroxymethyl)-6-[2-oxo-2-(1,3-thiazol-2-ylamino)ethyl]oxan-3-yl]-3-piperidin-1-ylpropanamide, N-[(4S,7R,8R)-8-methoxy-4,7,10-trimethyl-11-oxo-5-(phenylmethyl)-2-oxa-5,10-diazabicyclo[10.4.0]hexadeca-1(12),13,15-trien-14-yl]butanamide, 2-[(2R,3R,6S)-3-[[(2,5-difluoroanilino)-oxomethyl]amino]-2-(hydroxymethyl)-3,6-dihydro-2H-pyran-6-yl]-N-[3-(4-morpholinyl)propyl]acetamide, N-benzyl-2-chloroquinazolin-4-amine, N-[(2R,3S,6S)-6-[2-[(4-fluorophenyl)sulfonylamino]ethyl]-2-(hydroxymethyl)oxan-3-yl]oxane-4-carboxamide, 5-amino-1-[2,6-dichloro-4-(trifluoromethyl)phenyl]-4-(trifluoromethane)sulfinyl-1H-pyrazole-3-carbonitrile, N-[(1S,3S,4aS,9aR)-1-(hydroxymethyl)-3-[2-oxo-2-(pyridin-2-ylmethylamino)ethyl]-3,4,4a,9a-tetrahydro-1H-pyrano[3,4-b][1]benzofuran-6-yl]cyclobutanecarboxamide, and 1-[[(8S,9R)-6-[(2S)-1-hydroxypropan-2-yl]-8-methyl-5-oxo-10-oxa-1,6,14,15-tetrazabicyclo[10.3.0]pentadeca-12,14-dien-9-yl]methyl]-1-methyl-3-(3-pyridin-2-yloxyphenyl)urea;

or pharmaceutically acceptable salts thereof, or solvates, tautomers, stereoisomers, and/or prodrugs of any of the foregoing.

Compounds useful in modulating proliferation (e.g., of neoplasias associated with the NF-κB pathway, of neoplasias associated with the Hippo pathway) are provided in Table 1.

TABLE 1
Representative compounds

| Compound | | |
|---|---|---|
| ID | Structure | Name |
| BRD- K96698997 (Cmpd. 1) | 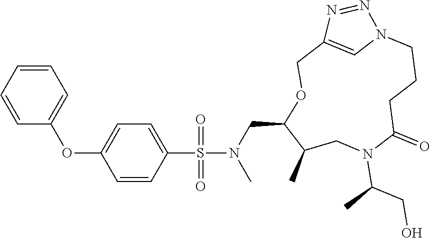 [SMILES: C[C@@H]1CN([C@H](C)CO)C(=O)CCCn2cc(nn2)CO[C@@H]1CN(C)S(=O)(=O)c1ccc(Oc2ccccc2)cc1] | N-[[(8R,9S)-6-[(2R)-1-hydroxypropan- 2-yl]-8-methyl-5-oxo-10-oxa- 1,6,13,14- tetrazabicyclo[10.2.1]pentadeca- 12(15),13-dien-9-yl]methyl]-N-methyl- 4-phenoxybenzenesulfonamide |
| BRD- K13719685 (Cmpd. 2) | 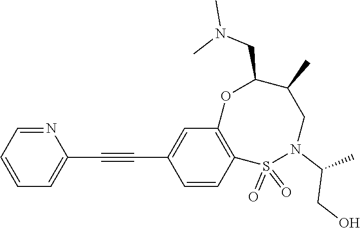 [SMILES: C[C@H](CO)N1C[C@H](C)[C@H](CN(C)C)Oc2cc(C#Cc3ccccn3)ccc2S1(=O)=O] | (4S,5R)-5-((dimethylamino)methyl)-2- ((R)-1-hydroxypropan-2-yl)-4-methyl- 8-(pyridin-2-ylethynyl)-2,3,4,5- tetrahydrobenzo[b][1,4,5]oxathiazocine 1,1-dioxide |
| BRD- K28862419 (Cmpd. 4) | 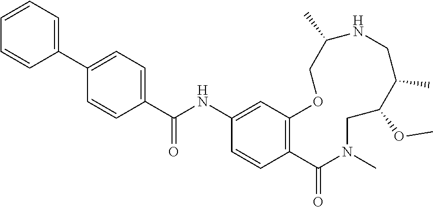 [SMILES: CO[C@@H]1CN(C)C(=O)c2ccc(NC(=O)c3ccc(-c4ccccc4)cc3)cc2OC[C@H](C)NC[C@@H]1C] | N-[(4S,7S,8S)-8-methoxy-4,7,10- trimethyl-11-oxo-2-oxa-5,10- diazabicyclo[10.4.0]hexadeca- 1(12),13,15-trien-15-yl]-4- phenylbenzamide |
| BRD- K34692511 (Cmpd. 3) | 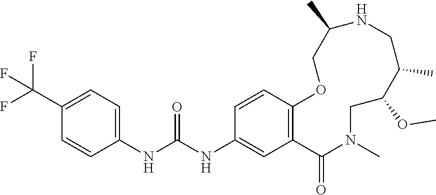 [SMILES: CO[C@@H]1CN(C)C(=O)c2cc(NC(=O)Nc3ccc(C(F)(F)F)cc3)ccc2OC[C@@H](C)NC[C@@H]1C] | 1-[(4R,7S,8S)-8-methoxy-4,7,10- trimethyl-11-oxo-2-oxa-5,10- diazabicyclo[10.4.0]hexadeca- 1(12),13,15-trien-14-yl]-3-[4- (trifluoromethyl)phenyl]urea |
| BRD- K70003473 (Cmpd. 5) | 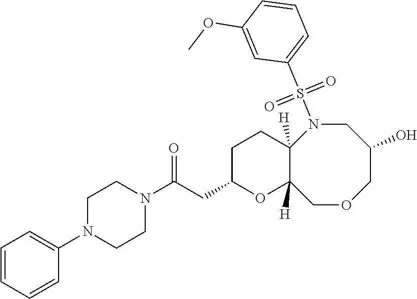 [SMILES: COc1cccc(S(=O)(=O)N2C[C@H](O)COC[C@@H]3O[C@H](CC(=O)N4CCN(c5ccccc5)CC4)CC[C@H]32)c1] | 2-[(3S,6aR,8S,10aR)-3-hydroxy-1-(3- methoxyphenyl)sulfonyl- 3,4,6,6a,8,9,10,10a-octahydro-2H- pyrano[2,3-c][1,5]oxazocin-8-yl]-1-(4- phenyl-1-piperazinyl)ethanone |
| BRD- K46678324 (Cmpd. 6) | 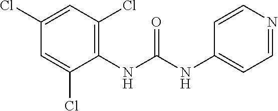 [SMILES: O=C(Nc1ccncc1)Nc1c(Cl)cc(Cl)cc1Cl] | 1-pyridin-4-yl-3-(2,4,6- trichlorophenyl)urea |
| BRD- K06593056 (Cmpd. 7) | 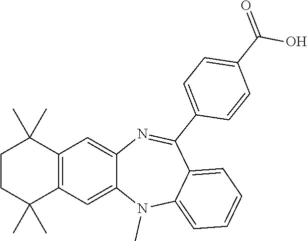 [SMILES: CN1c2cc3c(cc2N=C(c2ccc(C(=O)O)cc2)c2ccccc21)C(C)(C)CCC3(C)C] | 4-(5,7,7,10,10-pentamethyl-8,9- dihydronaphtho[2,3- b][1,4]benzodiazepin-13-yl)benzoic acid |
| BRD- A61154809 (Cmpd. 8) | 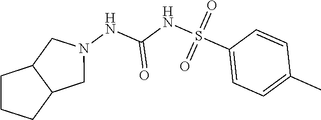 [SMILES: Cc1ccc(S(=O)(=O)NC(=O)NN2CC3CCCC3C2)cc1] | 1-(3,3a,4,5,6,6a-hexahydro-1H- cyclopenta[c]pyrrol-2-yl)-3-(4- methylphenyl)sulfonylurea |
| BRD- K77793136 (Cmpd. 9) | 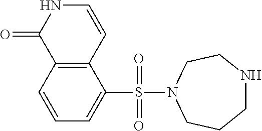 [SMILES: O=c1[nH]ccc2c(S(=O)(=O)N3CCCNCC3)cccc12] | 5-(1,4-diazepan-1-ylsulfonyl)-2H- isoquinolin-1-one |
| BRD- K15567136 (Cmpd. 10) | 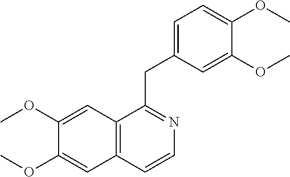 | 1-[(3,4-dimethoxyphenyl)methyl]- 6,7-dimethoxyisoquinoline |
| BRD- K88429204 (Cmpd. 11) | 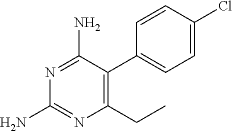 [SMILES: CCc1nc(N)nc(N)c1-c1ccc(Cl)cc1] | 5-(4-chlorophenyl)-6- ethylpyrimidine-2,4-diamine |
| BRD- K42095107 (Cmpd. 12) | 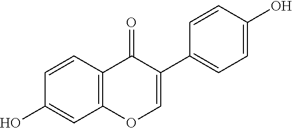 [SMILES: O=c1c(-c2ccc(O)cc2)coc2cc(O)ccc12] | 7-hydroxy-3-(4- hydroxyphenyl)chromen-4-one |
| BRD- K43796186 (Cmpd. 13) | 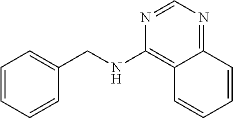 [SMILES: c1ccc(CNc2ncnc3ccccc23)cc1] | N-benzylquinazolin-4-amine |
| BRD- K37451830 (Cmpd. 14) | 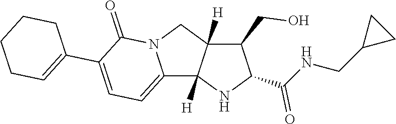 [SMILES: O=C(NCC1CC1)[C@@H]1N[C@@H]2c3ccc(C4=CCCCC4)c(=O)n3C[C@@H]2[C@H]1CO] | (2R,3R,3aS,9bS)-7-(1-cyclohexenyl)- N-(cyclopropylmethyl)-3- (hydroxymethyl)-6-oxo-1,2,3,3a,4,9b- hexahydropyrrolo[2,3-a]indolizine-2- carboxamide |
| BRD- K03953354 (Cmpd. 15) | 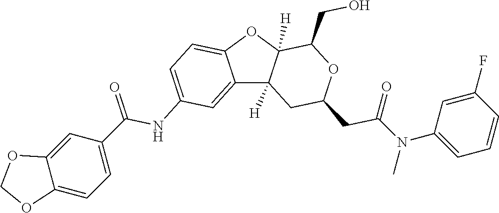 [SMILES: CN(C(=O)C[C@H]1C[C@H]2c3cc(NC(=O)c4ccc5c(c4)OCO5)ccc3O[C@H]2[C@@H](CO)O1)c1cccc(F)c1] | N-[(1R,3R,4aS,9aR)-3-[2-[(3- fluorophenyl)methylamino]-2- oxoethyl]-1-(hydroxymethyl)- 3,4,4a,9a-tetrahydro-1H-pyrano[3,4- b]benzofuran-6-yl]-1,3-benzodioxole- 5-carboxamide |
| BRD- K39839146 (Cmpd. 16) | 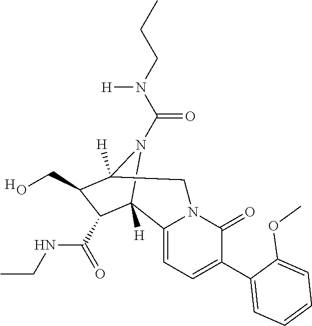 | (1S,9R,10R,11R)-11-N-ethyl-10- (hydroxymethyl)-5-(2- methoxyphenyl)-6-oxo-12-N-propyl- 7,12-diazatricyclo[7.2.1.02,7]dodeca- 2,4-diene-11,12-dicarboxamide |
| BRD- K62768599 (Cmpd. 17) | 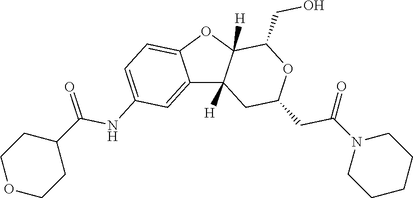 [SMILES: O=C(Nc1ccc2c(c1)[C@H]1C[C@@H](CC(=O)N3CCCCC3)O[C@@H](CO)[C@H]1O2)C1CCOCC1] | N-[(1S,3S,4aR,9aS)-1- (hydroxymethyl)-3-[2-oxo-2-(1- piperidinyl)ethyl]-3,4,4a,9a-tetrahydro- 1H-pyrano[3,4-b]benzofuran-6-yl]-4- oxanecarboxamide |
| BRD- K42367391 (Cmpd. 18) | 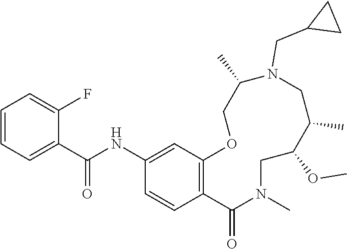 [SMILES: CO[C@@H]1CN(C)C(=O)c2ccc(NC(=O)c3ccccc3F)cc2OC[C@H](C)N(CC2CC2)C[C@@H]1C] | N-[(5S,6S,9S)-8-(cyclopropylmethyl)- 5-methoxy-3,6,9-trimethyl-2-oxo-11- oxa-3,8-diazabicyclo[10.4.0]hexadeca- 1(12),13,15-trien-14-yl]-2- fluorobenzamide |
| BRD- K22874335 (Cmpd. 19) | 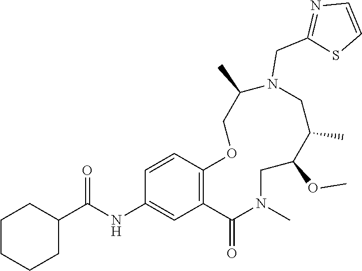 [SMILES: CO[C@H]1CN(C)C(=O)c2cc(NC(=O)C3CCCCC3)ccc2OC[C@@H](C)N(Cc2nccs2)C[C@@H]1C] | N-[(4R,7S,8R)-8-methoxy-4,7,10- trimethyl-11-oxo-5-(1,3-thiazol-2- ylmethyl)-2-oxa-5,10- diazabicyclo[10.4.0]hexadeca- 1(12),13,15-trien-14- yl]cyclohexanecarboxamide |
| BRD- K41723088 (Cmpd. 20) | 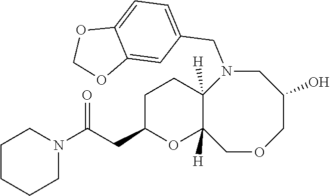 [SMILES: O=C(C[C@H]1CC[C@@H]2[C@H](COC[C@@H](O)CN2Cc2ccc3c(c2)OCO3)O1)N1CCCCC1] | 2-[(3S,6aR,8R,10aR)-1-(1,3- benzodioxol-5-ylmethyl)-3-hydroxy- 3,4,6,6a,8,9,10,10a-octahydro-2H- pyrano[2,3-c][1,5]oxazocin-8-yl]-1- piperidin-1-ylethanone |
| BRD- K68530167 (Cmpd. 21) | 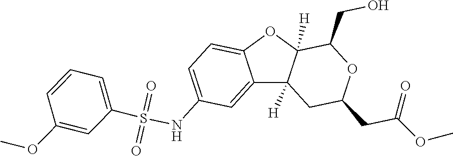 | 2-[(1R,3R,4aS,9aR)-1- (hydroxymethyl)-6-[(3- methoxyphenyl)sulfonylamino]- 3,4,4a,9a-tetrahydro-1H-pyrano[3,4- b]benzofuran-3-yl]acetic acid methyl ester |
| BRD- K00135177 (Cmpd. 24) | 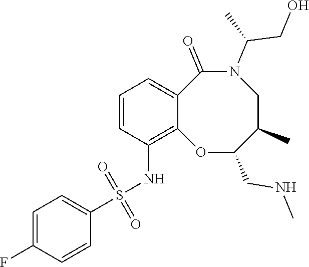 [SMILES: CNC[C@@H]1Oc2c(NS(=O)(=O)c3ccc(F)cc3)cccc2C(=O)N([C@H](C)CO)C[C@H]1C] | 4-fluoro-N-[(2R,3R)-5-[(2R)-1- hydroxypropan-2-yl]-3-methyl-2- (methylaminomethyl)-6-oxo-3,4- dihydro-2H-1,5-benzoxazocin-10- yl]benzenesulfonamide |
| BRD- K11266478 (Cmpd. 23) | 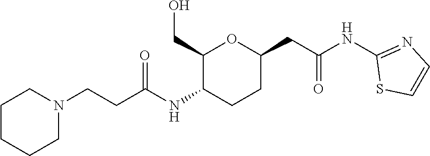 [SMILES: O=C(C[C@H]1CC[C@H](NC(=O)CCN2CCCCC2)[C@@H](CO)O1)Nc1nccs1] | N-[(2S,3S,6R)-2-(hydroxymethyl)-6- [2-oxo-2-(1,3-thiazol-2- ylamino)ethyl]oxan-3-yl]-3-piperidin- 1-ylpropanamide |
| BRD- K22754756 (Cmpd. 22) | 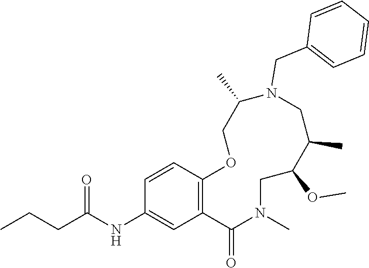 [SMILES: CCCC(=O)Nc1ccc2c(c1)C(=O)N(C)C[C@H](OC)[C@H](C)CN(Cc1ccccc1)[C@@H](C)CO2] | N-[(4S,7R,8R)-8-methoxy-4,7,10- trimethyl-11-oxo-5-(phenylmethyl)-2- oxa-5,10- diazabicyclo[10.4.0]hexadeca- 1(12),13,15-trien-14-yl]butanamide |
| BRD- K40143134 (Cmpd. 25) | 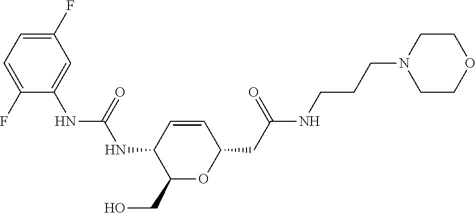 [SMILES: O=C(C[C@H]1C=C[C@@H](NC(=O)Nc2cc(F)ccc2F)[C@H](CO)O1)NCCCN1CCOCC1] | 2-[(2R,3R,6S)-3-[[(2,5- difluoroanilino)-oxomethyl]amino]-2- (hydroxymethyl)-3,6-dihydro-2H- pyran-6-yl]-N-[3-(4- morpholinyl)propyl]acetamide |
| BRD- K11758216 (Cmpd. 26) | 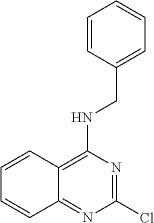 [SMILES: Clc1nc(NCc2ccccc2)c2ccccc2n1] | N-benzyl-2-chloroquinazolin-4-amine |
| BRD- K48052543 (Cmpd. 27) | 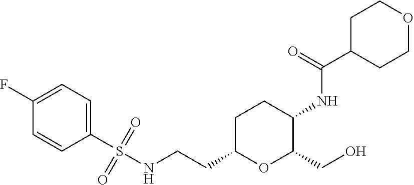 [SMILES: O=C(N[C@H]1CC[C@@H](CCNS(=O)(=O)c2ccc(F)cc2)O[C@H]1CO)C1CCOCC1] | N-[(2R,3S,6S)-6-[2-[(4- fluorophenyl)sulfonylamino]ethyl]-2- (hydroxymethyl)oxan-3-yl]oxane-4- carboxamide |
| BRD- A50675702 (Cmpd. 28) | 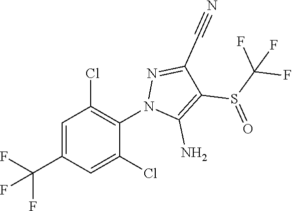 [SMILES: N#Cc1nn(-c2c(Cl)cc(C(F)(F)F)cc2Cl)c(N)c1S(=O)C(F)(F)F] | 5-amino-1-[2,6-dichloro-4- (trifluoromethyl)phenyl]-4- (trifluoromethane)sulfinyl-1H- pyrazole-3-carbonitrile |
| BRD- K28043081 (Cmpd. 29) | 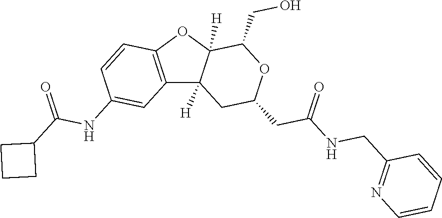 [SMILES: O=C(C[C@@H]1C[C@H]2c3cc(NC(=O)C4CCC4)ccc3O[C@H]2[C@H](CO)O1)NCc1ccccn1] | N-[(1S,3S,4aS,9aR)-1- (hydroxymethyl)-3-[2-oxo-2-(pyridin- 2-ylmethylamino)ethyl]-3,4,4a,9a- tetrahydro-1H-pyrano[3,4- b][1]benzofuran-6- yl]cyclobutanecarboxamide |
| BRD- K19969618 (Cmpd. 30) | 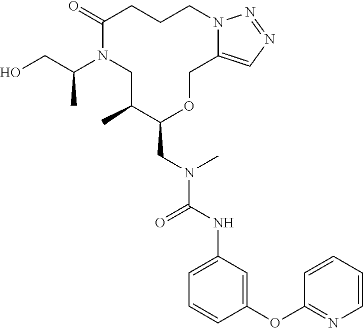 [SMILES: C[C@H]1CN([C@@H](C)CO)C(=O)CCCn2nncc2CO[C@H]1CN(C)C(=O)Nc1cccc(Oc2ccccn2)c1] | 1-[[(8S,9R)-6-[(2S)-1-hydroxypropan- 2-yl]-8-methyl-5-oxo-10-oxa- 1,6,14,15- tetrazabicyclo[10.3.0]pentadeca-12,14- dien-9-yl]methyl]-1-methyl-3-(3- pyridin-2-yloxyphenyl)urea |

It will be understood that in the event of any inconsistency between the chemical formula, Compound Name, and BRD Name in any table herein including Tables 1, 3, and 4, each compound will be considered part of the present disclosure.

Pharmaceutical Therapeutics

Compounds that modulate the Hippo and/or NF-κB pathways are useful in the methods of the invention. For example, compounds that modulate the Hippo and/or NF-κB pathways are useful for the treatment of proliferative diseases, such as cancer (e.g., sarcoma, pancreas, prostate, head and neck, liver, and breast cancer).

In one embodiment, agents discovered to have medicinal value using the methods described herein are useful as a drug or as information for structural modification of existing compounds, e.g., by rational drug design. Such methods are useful for screening agents having an effect on Hippo and/or NF-κB pathway modulation.

For therapeutic uses, the compositions or agents identified using the methods disclosed herein may be administered systemically, for example, formulated in a pharmaceutical composition such as those containing a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the disease. Generally, amounts will be in the range of those used for other agents used in the treatment of other diseases associated with, although in certain instances lower amounts will be needed because of the increased specificity of the compound. A compound is administered at a dosage that is determined by a method known to one skilled in the art or using any assay that measures the modulation of the Hippo and/or NF-κB pathways.

Pharmaceutical Compositions

The administration of a compound for the treatment of a disease associated with modulation of the Hippo and/or NF-κB pathways may be by any suitable means that results in a concentration of the therapeutic that, combined with other components, is effective in ameliorating, reducing, or stabilizing the disease. Provided herein are compounds for the treatment of a disease. Additionally, the compounds of the present disclosure may be for the preparation of a medicament for the treatment of a disease.

Any compound of the present disclosure may be formulated in a pharmaceutical composition comprising the compound and one or more pharmaceutically acceptable carriers, diluents, or carriers. The compound may be contained in any appropriate amount in any suitable carrier substance and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 μg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/Kg body weight. In other embodiments, it is envisaged that doses may be in the range of about 5 mg compound/Kg body to about 20 mg compound/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target the Hippo and/or NF-κB pathways by using carriers or chemical derivatives to deliver the therapeutic agent to a particular targeted site (e.g., cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added. The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a disease, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution-enhancing or solubilizing agent can be added, or the solvent may include 10-60% w/w of propylene glycol or the like.

Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Solid Dosage Forms for Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated, or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active therapeutic substance). The coating may be applied on the solid dosage form in a similar manner as that described in Encyclopedia of Pharmaceutical Technology, supra.

At least two therapeutics may be mixed together in the tablet or may be partitioned. In one example, the first active therapeutic is contained on the inside of the tablet, and the second active therapeutic is on the outside, such that a substantial portion of the second therapeutic is released prior to the release of the first therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

Controlled Release Oral Dosage Forms

Controlled release compositions for oral use may, e.g., be constructed to release the active therapeutic by controlling the dissolution and/or the diffusion of the active substance. Dissolution or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, camauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

Methods of Treatment

The disclosure includes a method of treating or preventing a proliferative disease, such as cancer, in a subject in need thereof. The methods of the disclosure may comprise administering to the subject a therapeutically effective amount of at least one compound of the invention, which is optionally formulated in a pharmaceutical composition. In certain embodiments, the method further comprises administering to the subject an additional therapeutic agent that treats or prevents cancer. The compound may be a compound for the treatment of a proliferative disease. In certain embodiments, the compound may be a compound for the manufacture of a medicament for the treatment of a hyperproliferative disease. The method for the treatment of a proliferative disease may comprise the administration of a compound or pharmaceutical composition as disclosed herein.

Provided here in are compounds for the treatment or prevention of a disease (e.g., a proliferative disease). In some embodiments, the Compound may a compound selected from Table 1, 2, 3, 4, or 5. The compound may also be for the preparation of a medicament for the treatment or prevention of a disease.

Dosage

The effective amount or dose of a compound of the present invention depends on the age, sex and weight of the patient, the current medical condition of the patient and the progression of a cancer in the patient being treated. The skilled artisan is able to determine appropriate dosages depending on these and other factors. For example, a suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. In some embodiments, the therapeutically effective amount may be from 0.01 to 1000 mg/kg subject (e.g., from 0.01 to 0.1 mg/kg, from 0.1 mg/kg to 1 mg/kg, from 1 mg/kg to 10 mg/kg, from 10 mg/kg to 100 mg/kg, from 100 mg/kg to 1000 mg/kg, etc.). The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses. In certain embodiments, the compound is administered daily, weekly, monthly, or multiple times per day, in an amount from 0.1 mg to 1000 mg (e.g., from 0.1 mg to 1 mg, from 1 mg to 10 mg, from 10 mg to 100 mg, from 100 mg to 1000 mg, etc.).

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of a compound of the invention is optionally given continuously; alternatively, the dose of drug being administered is temporarily reduced or temporarily suspended for a certain length of time. The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 6%, 70%, 75%, 80%, 85%, 90%, 95%, or 100%.

Once improvement of the subject's conditions has occurred, a maintenance dose may be administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

Kits or Pharmaceutical Systems

The present compositions may be assembled into kits or pharmaceutical systems for use in ameliorating a disease associated with modulating the Hippo and/or NF-κB pathways. Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the agents of the invention. Kits of the invention include at least one or more agents that modulate the Hippo and/or NF-κB pathways. If desired, the kit also includes an additional agent. Optionally, the kit includes instructions for administering the agent in combination with one or more agents that bind to a binding site or that reduce activity, thereby increasing the efficacy of the agent relative to the efficacy of the agent administered alone. Methods for measuring the efficacy of agents are known in the art and are described herein (e.g., measuring the IC₅₀).

Combination Therapies

The compounds and pharmaceutical compositions can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will consider compatibility of the desired therapeutics and/or procedures and the desired therapeutic effect to be achieved. It will also be appreciated that the therapies employed may achieve a desired effect for the same disorder, or they may achieve different effects (e.g., control of any adverse effects).

In some embodiments, anti-cancer compounds are coadministered with other anti-cancer therapies. Examples of other anti-cancer therapies include chemotherapy, radiotherapy, gene therapy, surgery, hormonal therapy, anti-angiogenic therapy, and immunotherapy. Other examples of drugs to combine with the compounds described herein include pharmaceuticals for the treatment of different, yet associated or related symptoms or indications. Combination methods can involve the use of the two (or more) agents formulated together or separately, as determined to be appropriate by those of skill in the art. In one example, two or more drugs are formulated together for the simultaneous or near simultaneous administration of the agents.

When the compositions of the present disclosure include a combination of a compound of the disclosure described herein and one or more additional therapeutic or prophylactic agents, both the compound and the additional agent can be present at dosage levels of between 1 to 100%, and more preferably between 5 to 95% of the dosage normally administered in a monotherapy regimen.

Screening Assays

As described herein, the disclosure provides specific examples of chemical compounds that modulate the Hippo and/or NF-κB pathways when administered alone or in combination with an additional agent. The disclosure further provides protocols for identifying agents (including nucleic acids, peptides, small molecule inhibitors, and mimetics) that are capable of modulating the Hippo and/or NF-κB pathways and/or verifying the activity of the agents described herein. Compounds identified are expected to be useful for the treatment or prevention of proliferative diseases such as cancer (e.g., sarcoma, pancreas, prostate, head and neck, liver, and breast cancer) or a disorder or symptom thereof.

Virtually any agent that specifically modulates Hippo and/or NF-κB pathways may be employed in the methods of the invention, particularly those involving screening. Methods of the invention are useful for the high-throughput low-cost screening of candidate agents that impact, positively or negatively, the Hippo and/or NF-κB pathways. A candidate agent that specifically modulates the Hippo and/or NF-κB pathways is then isolated and tested for activity in an in vitro assay or in vivo assay for its ability to modulate the Hippo and/or NF-κB pathways. One skilled in the art appreciates that the effects of a candidate agent on the Hippo and/or NF-κB pathways is typically compared to a corresponding control not contacted with the candidate agent. Thus, the screening methods include comparing the Hippo and/or NF-κB pathways contacted by a candidate agent to those of an untreated control.

In other embodiments, the expression or activity of a Hippo and/or NF-κB pathway treated with a candidate agent is compared to untreated control to identify a candidate compound that modulates the Hippo and/or NF-κB pathways. Polypeptide expression or activity can be compared by procedures well known in the art, such as Western blotting, flow cytometry, immunocytochemistry, binding to magnetic and/or specific antibody-coated beads, in situ hybridization, fluorescence in situ hybridization (FISH), ELISA, microarray analysis, RT-PCR, Northern blotting, or colorimetric assays, such as the Bradford Assay and Lowry Assay.

In one working example, one or more candidate agents are added at varying concentrations. An agent that modulates the Hippo and/or NF-κB pathways is considered useful in the invention; such an agent may be used, for example, as a therapeutic to prevent, delay, ameliorate, stabilize, or treat proliferative diseases such as cancer (e.g., sarcoma, pancreas, prostate, head and neck, liver, and breast cancer) or a disorder or symptom thereof. An agent identified according to a method of the invention is locally or systemically delivered to treat such a disease in situ.

In one embodiment, the effect of a candidate agent may, in the alternative, be measured at the level of polypeptide production using the same general approach and standard immunological techniques, such as Western blotting or immunoprecipitation with an antibody specific for the specific target. For example, immunoassays may be used to detect or monitor the expression of particular Hippo and/or NF-κB pathway members. In one embodiment, the invention identifies a polyclonal or monoclonal antibody (produced as described herein) that is capable of binding to a binding site and reducing the biological activity of a polypeptide. A compound that reduces the expression or activity of a polypeptide is considered particularly useful. Again, such an agent may be used, for example, as a therapeutic to prevent or treat proliferative diseases such as cancer (e.g., sarcoma, pancreas, prostate, head and neck, liver, and breast cancer) or a disorder or symptom thereof.

Alternatively, or in addition, candidate compounds may be identified by first assaying those that specifically modulate the Hippo and/or NF-κB pathways of the invention and subsequently testing their effect as described in the Examples. In one embodiment, the efficacy of a candidate agent is dependent upon its ability to interact with the polypeptide. Such an interaction can be readily assayed using any number of standard binding techniques and functional assays (e.g., those described in the Materials and Methods Examples). A candidate compound may be tested in vitro for interaction and binding with the Hippo and/or NF-κB pathways and its ability to modulate may be assayed by any standard assays (e.g., those described herein). In one embodiment, modulating the Hippo and/or NF-κB pathways is determined by assaying using flow cytometry analysis. In another embodiment, expression is monitored immunohistochemically.

Potential agents include organic molecules, peptides, peptide mimetics, polypeptides, nucleic acid ligands, aptamers, and antibodies that modulate the Hippo and/or NF-κB pathways. In one particular example, a candidate compound that modulates the Hippo and/or NF-κB pathways may be identified using a chromatography-based technique. For example, a recombinant polypeptide of the invention may be purified by standard techniques from cells engineered to express the polypeptide, or may be chemically synthesized, once purified the peptide is immobilized on a column. A solution of candidate agents is then passed through the column, and an agent that specifically modulates the Hippo and/or NF-κB pathways is identified on the basis of its ability to bind to polypeptide and to be immobilized on the column. To isolate the agent, the column is washed to remove non-specifically bound molecules, and the agent of interest is then released from the column and collected. Agents isolated by this method (or any other appropriate method) may, if desired, be further purified (e.g., by high performance liquid chromatography). In addition, these candidate agents may be tested for their ability to modulate the Hippo and/or NF-κB pathways. Agents isolated by this approach may also be used, for example, as therapeutics to treat or prevent particular diseases. Compounds that are identified as binding to a polypeptide with an affinity constant less than or equal to 1 nM, 5 nM, 10 nM, 100 nM, 1 μM or 10 μM are considered particularly useful in the invention.

Methods for Characterizing Compounds

The methods for identifying agents useful in the invention include, but are not limited to, those described in the examples. For example, in some embodiments, agents are identified and characterized by interrogating their intervention in RIT1-mutant lung cancer using, for example, the PC9-RIT1^{M901} small molecule drug screen described in Vichas et al., bioRxiv preprint doi.org/10.1101/2020.07.03.187310, posted Jul. 8, 2020, incorporated herein by reference.

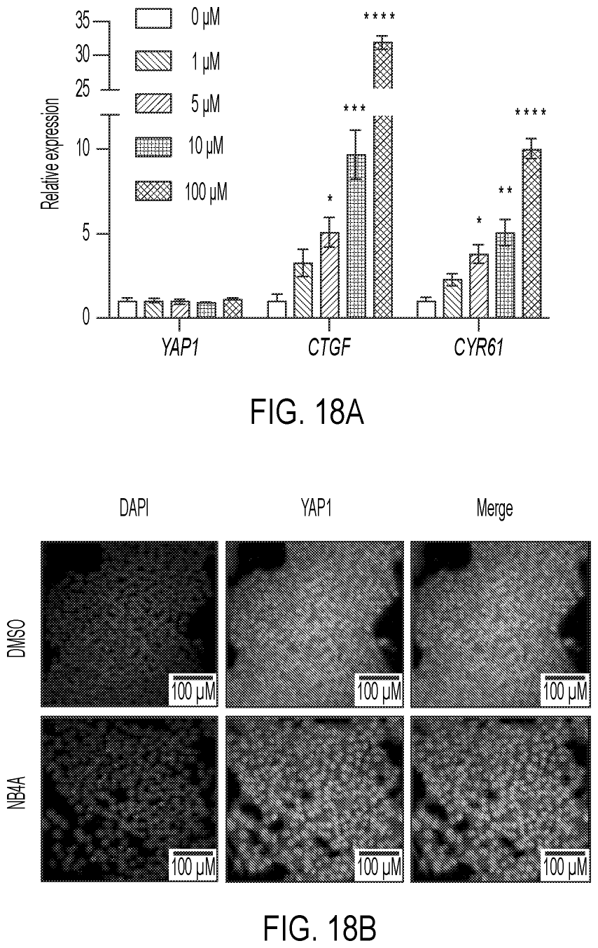

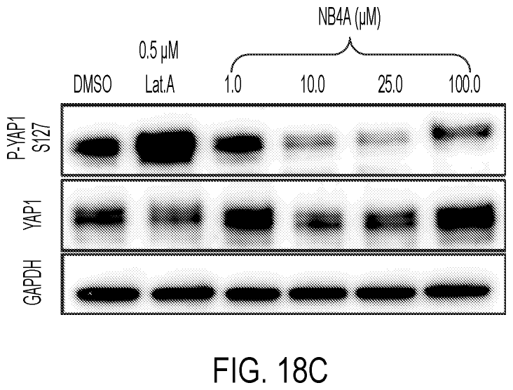

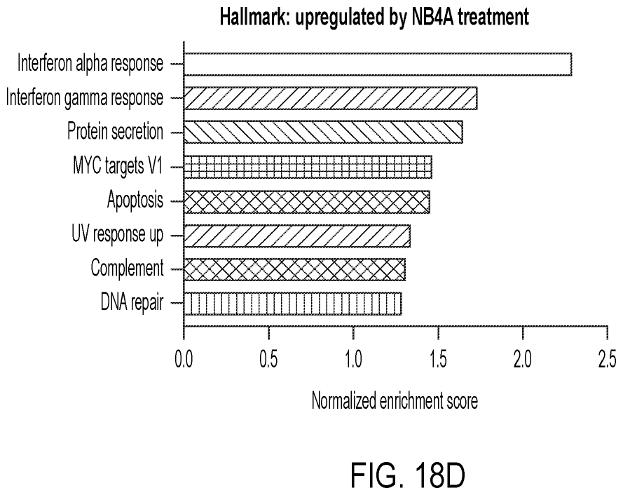

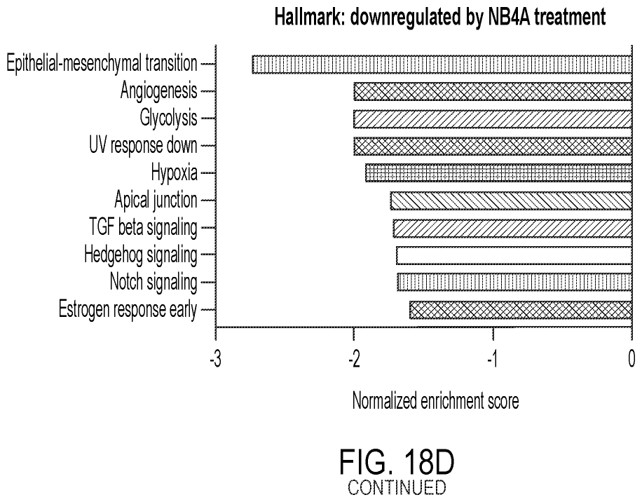

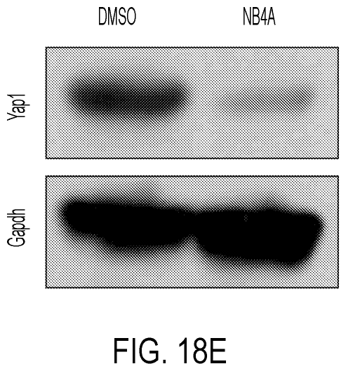

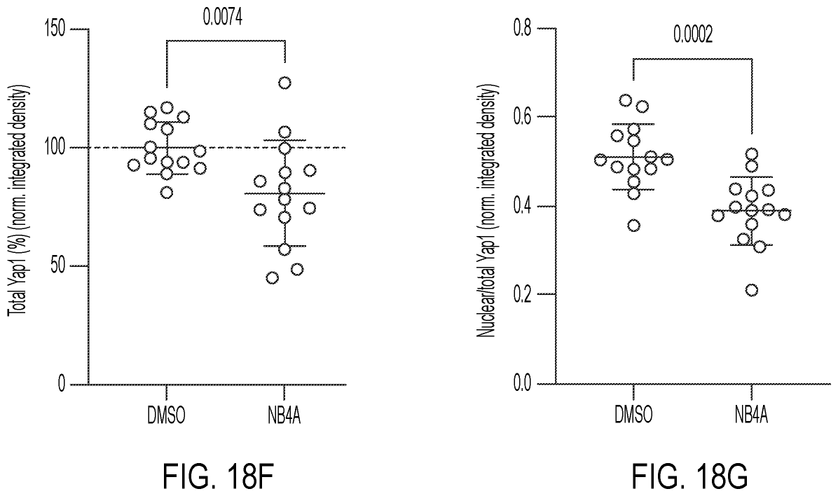

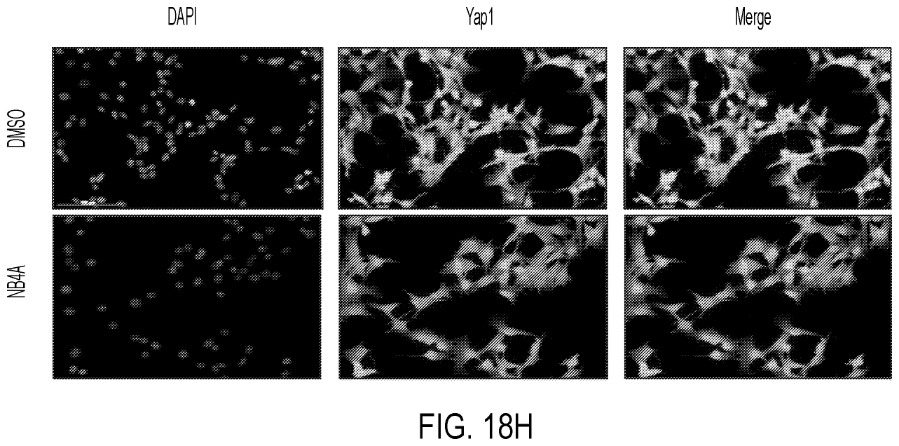

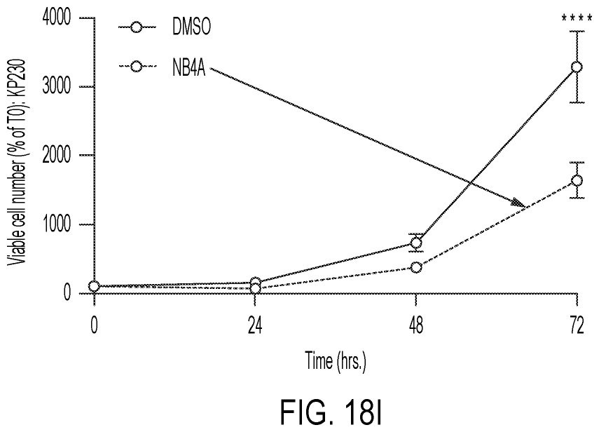

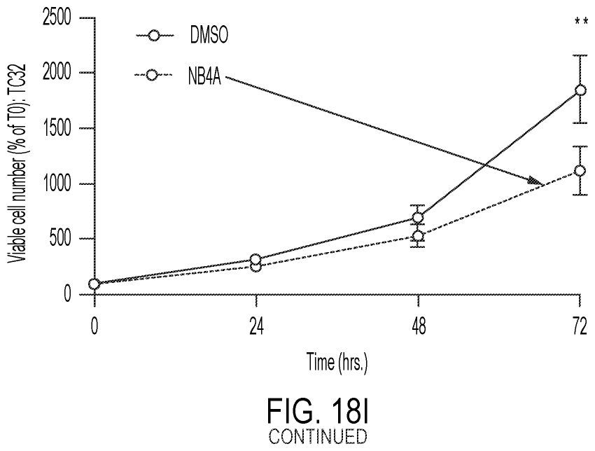

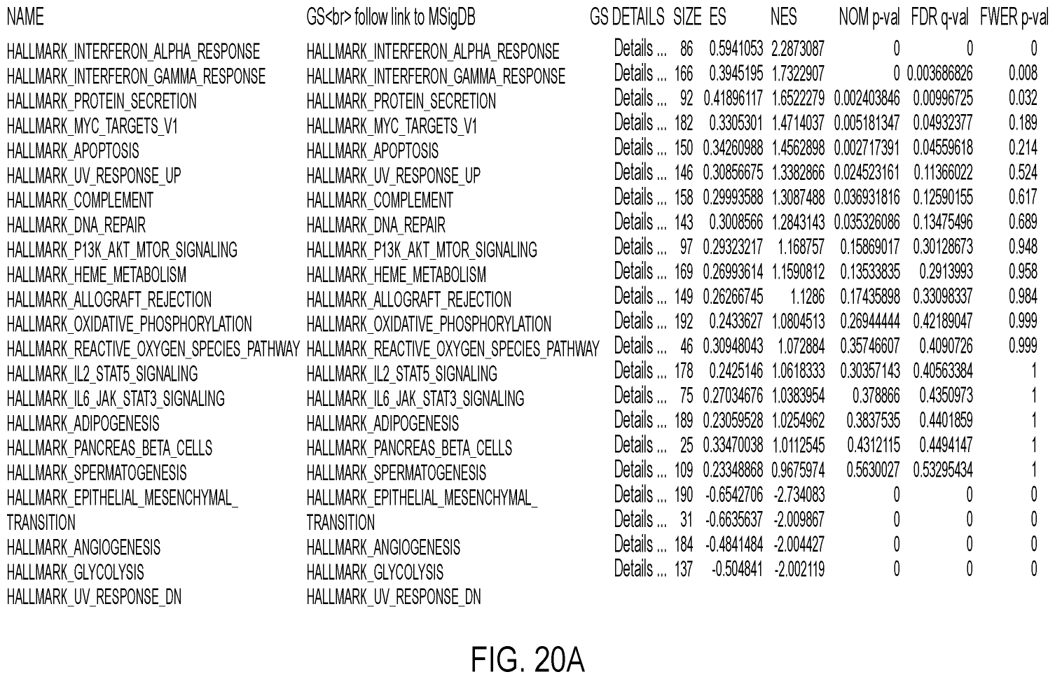

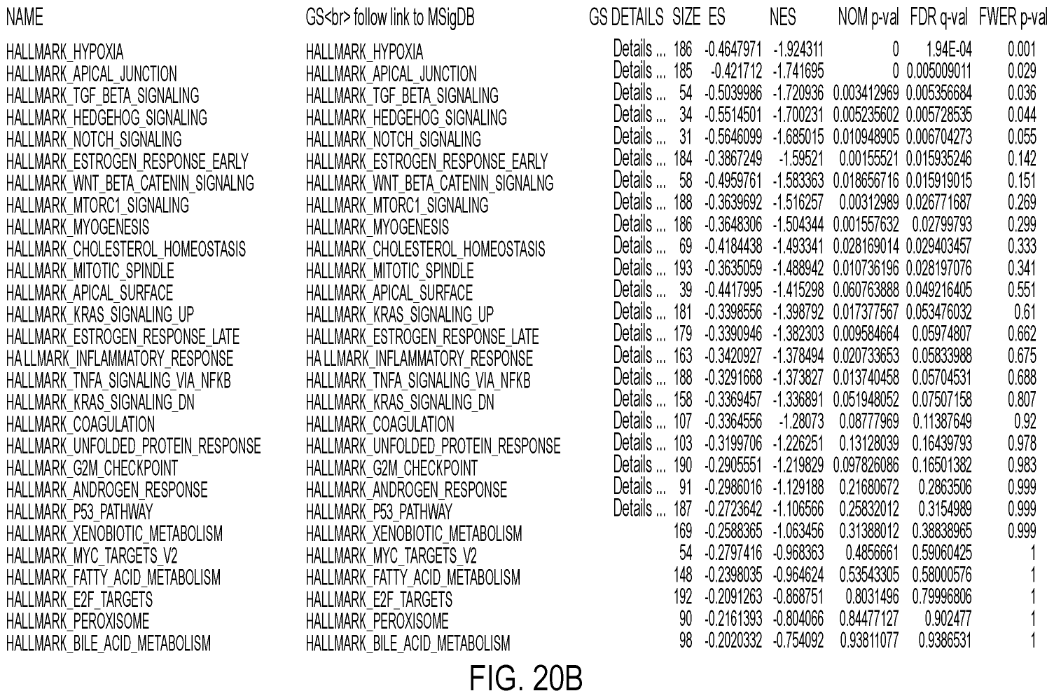

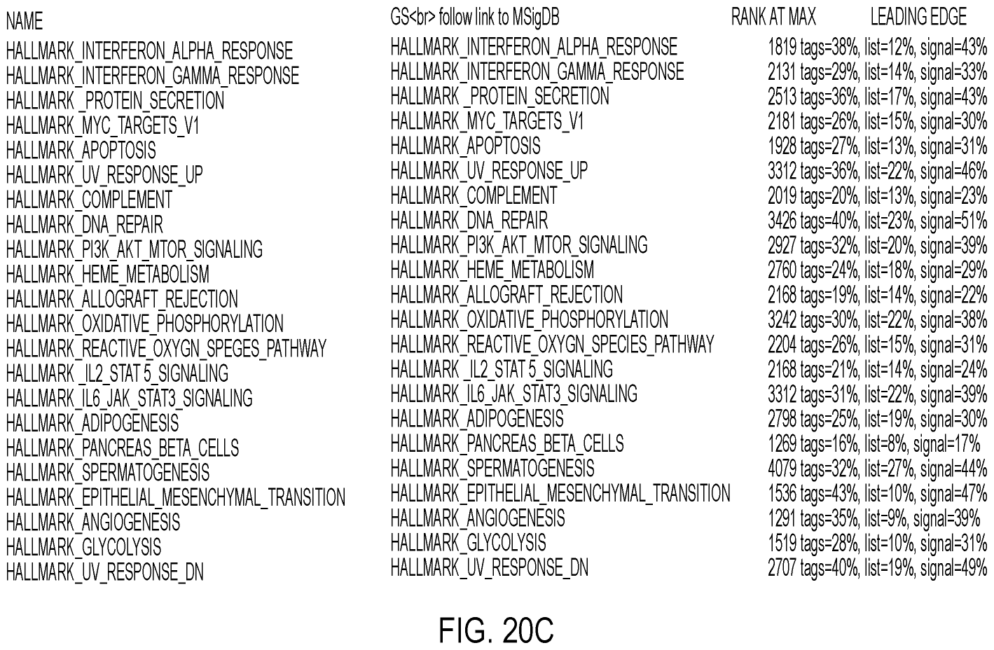

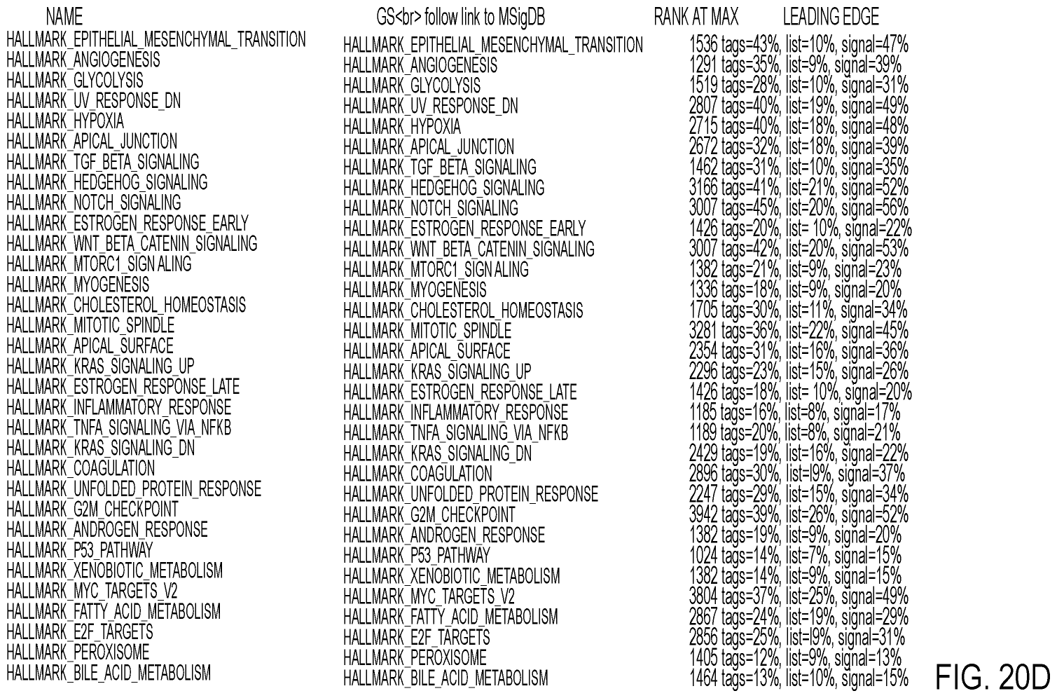

In some embodiments, the compounds of the present disclosure may be characterized by the regulation of certain hallmark genes associated with pathways (following administration of a compound of the present disclosure to a neoplasia associated with the NF-κB and/or Hippo signaling pathway). For example, the compounds of the present disclosure may be characterized as up- or down-regulating any pathway as identified in FIGS. 18D and 20A-D. Modulation of these genes may reduce Yap1 protein levels (e.g., after 72 hours of treatment) and/or attenuate Yap1 nuclear localization. In certain implementations, the compounds may be characterized as reducing Yap1 ability to impact transcription.

Drug Treatment and Proliferation Analysis

In brief, for proliferation assays, cells are plated in 384-well plates at a density of 800 cells per well in 40 μL total volume. One day later, a serial dilution of each inhibitor is performed using a D300e dispenser (Tecan). 96 hours post-treatment, cell viability is determined using CellTiterGlo reagent (Promega) and luminescence quantified on an Envision MultiLabel Plate Reader (PerkinElmer). To calculate the fraction cell viability drug-treated cells are normalized to average cell viability of DMSO-only treated cells. Curve fitting is performed using GraphPad Prism four parameter inhibitor response with variable slope. AUC values were calculated by GraphPad Prism (Graphpad).

In some embodiments, agents are characterized by interrogating their role in promoting skeletal development by regulating osteoblast activity, osteoclast-mediated remodeling, and matrix composition, as described in Kegelman et al. FASEB J. 2018 May; 32(5): 2706-2721, incorporated herein by reference.

In some embodiments, agents are characterized by interrogating their role in neovascular function and transcriptional cytoskeletal feedback as a key regulator of cell motility in the YAP1/TAZ-Rho-ROCK-myosin II feedback axis as described in Mason et al., J. Cell Biol. 2019 Vol. 218 No. 4 1369-1389, incorporated herein by reference.

In some embodiments, agents are characterized by interrogating their role in promoting the expansion and differentiation of periosteal osteoblast precursors to accelerate bone fracture healing as described in Kegelman et al., bioRxiv preprint doi.org/10.1101/2020.03.17 995761 posted Mar. 19, 2020, incorporated herein by reference.

In some embodiments, agents are characterized by interrogating their role in T cell activation and proliferation as described in Stampouloglou et al., PLoS Biol 18(1): e3000591, doi.org/10.1371/journal.pbio.3000591.

In brief, cells may be cultured and stimulated in 96-well plates at 1×10⁵ cells per well. Plates are coated with anti-CD3 antibody (Biolegend) at concentrations of 0, 0.125, 0.25, 0.5, and 1 μg/mL at 4° C. overnight and are washed twice with PBS before incubation. Cells are stimulated in the anti-CD3-coated plates with soluble anti-CD28 at 2 μg/mL (Biolegend). On days 1 and 3, cells are stained with dead cell dye as well as antibodies recognizing the lineage and activation markers CD3 BUV737 (BD), CD4 BUV395 (BD), CD8 PerCP-Cy5.5 (Biolegend), CD69 PE (Biolegend), CD44 BV650 (Biolegend), and CD25 APC (Biolegend). For proliferation assays, CD4+ or CD8+ T cells are isolated and stained using the CellTrace Violet or CFSE Cell Proliferation Kit (Life Technologies). Briefly, purified cells are washed with PBS and incubated with CellTrace dye for 20 minutes at 37° C. protected from light. After 20 minutes, complete RMPI medium is added to the cell suspension, and the cells are incubated 5 minutes further before being washed and resuspended in complete RPMI medium. Cells are cultured in 96-well plates at 1×10⁵ cells per well and were stimulated using anti-CD3/CD28 dynabeads (Gibco) at a 1:1 ratio with T cells. On days 1 and 3, cells are stained with dead cell dye, and proliferation is measured at the same time.

Test Compounds and Extracts

In general, agents that modulate the Hippo and/or NF-κB pathways may be identified from large libraries of natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Agents used in screens may include known those known as therapeutics for the treatment of proliferative diseases such as cancer (e.g., sarcoma, pancreas, prostate, head and neck, liver, and breast cancer) or a disorder or symptom thereof. Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceanographic Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Nat. Acad. Sci. U.S.A. 90:6909, 1993; Erb et al., Proc. Nat. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994, each of which are hereby incorporated by reference in their entirety. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to have activity, further fractionation of the positive lead extract is necessary to isolate molecular constituents responsible for the observed effect. Thus, the goal of the extraction, fractionation, and purification process is the careful characterization and identification of a chemical entity within the crude extract that modulates the Hippo and/or NF-κB pathways. Methods of fractionation and purification of such heterogenous extracts are known in the art. If desired, compounds shown to be useful as therapeutics are chemically modified according to methods known in the art.

The practice of the present invention may employ, unless otherwise indicated, conventional techniques of molecular biology (including recombinant techniques), microbiology, cell biology, microscopy, computational biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Morphological Profiles from Cell Painting

Morphological profiling was tested using overexpression in human cells as a general approach to annotate gene and allele function (see Rohban et al., eLife 2017; 6:e24060, incorporated herein by reference in its entirety). First, a reference series of well-known genes was profiled, and a small number of variants thereof, by Cell Painting (see Bray et al., Nat Protoc. 2016 September; 11(9): 1757-1774, incorporated herein by reference in its entirety). In particular, whether the information content of this strategy would outweigh potential limitations (e.g., due to cellular context or expression level) was to be addressed. It was found that the approach successfully clustered genes and alleles based on functional similarity, revealed specific morphological changes even when present in only a subpopulation of heterogeneous cells, and uncovered novel functional connections between important biological pathways.

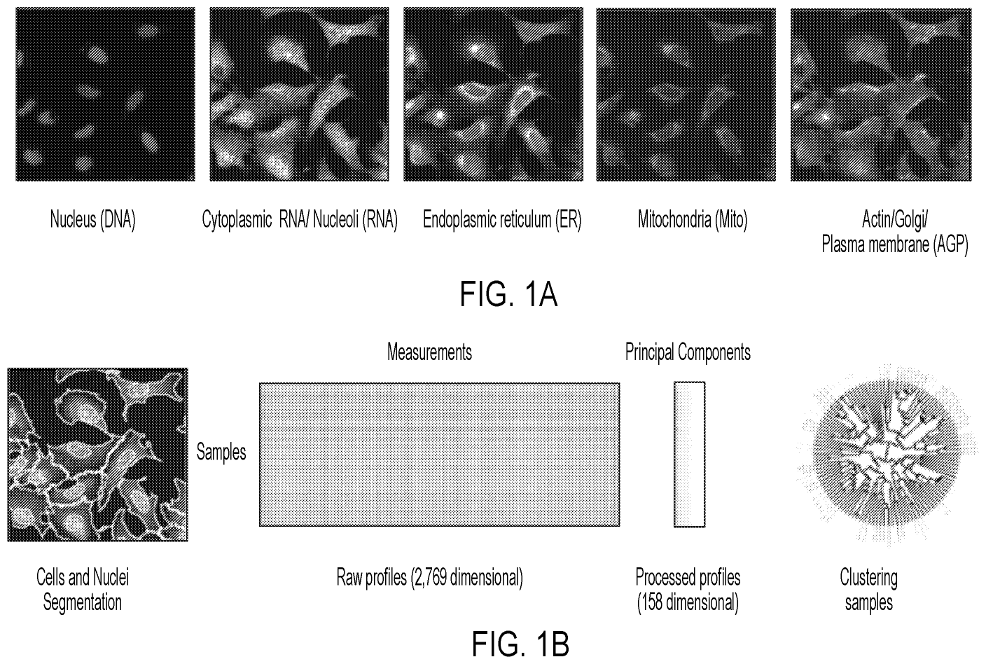

To profile each exogenously expressed gene (or allele therein), the previously developed image-based profiling assay, called Cell Painting was used (see Bray et al., Nat. Protoc. 2016 September; 11(9): 1757-1774). This microscopy-based assay consists of six stains imaged in five channels revealing eight cellular components: DNA, mitochondria, endoplasmic reticulum, Golgi, cytoplasmic RNA, nucleoli, actin, and plasma membrane (FIG. 1A). In five replicates in 384-well plate format, U2OS cells (human bone osteosarcoma cells), chosen for their flat morphology and previous validation in the assay, were infected with an arrayed ‘reference’ expression library of 323 open reading frame (ORF) constructs of partially characterized functions, a subset of which have been previously described. Of these, analysis of the 220 constructs that were most closely representative of the annotated full-length transcripts was prioritized. Morphological profiles were extracted using CellProfiler for image processing, yielding 1384 morphological features per cell, and Python/R scripts for data processing, including feature selection and dimensionality reduction (FIG. 1B). This computational pipeline yielded a 158-dimensional profile for each of 5 replicates for each gene or allele tested. Not all genes are likely to impact cellular morphology given the limitation of the experiment; using a single cell line at a single time point under a single set of conditions and stained with six fluorescent labels.

Next, the pathways that would be most and least likely to yield detectable morphological phenotypes were examined, recognizing that ‘pathways’ are neither separate nor well-defined entities. It was found that genes manually annotated as being in the Hippo, Hedgehog, cytoskeletal reorganization, and Mitogen-activated protein kinases (MAPK) pathways were more likely to result in a phenotype, whereas genes annotated as belonging to the Janus kinase/(signal transducer and activator of transcription proteins) (JAK/STAT), hypoxia, and bone morphogenetic protein (BMP) pathways were among the least likely to yield a phenotype under the conditions tested. Nevertheless, the majority of pathways could be interrogated by morphological profiling.

Relationship Between the Hippo Pathway and Regulators of NF-κB Signaling

Following additional proof of concept experiments, whether novel relationships might emerge from the unbiased classification of gene and allele function based on morphologic profiling was examined. By adopting a two-pronged approach, merging Cell Painting morphological analysis with transcriptional data, an unexpected relationship in human cells between two major signaling pathways, Hippo and NF-κB, both under intense study recently for their involvement in cancer, was identified. Through validation of these clustered genes, it has been identified that YAP1/TAZ-directed transcription is negatively regulated by NF-κB pathway effectors and without wishing to be bound by theory, the data suggests a novel regulatory mechanism that is independent of upstream Hippo kinases.

### Example 2: Data Mining for Morphology

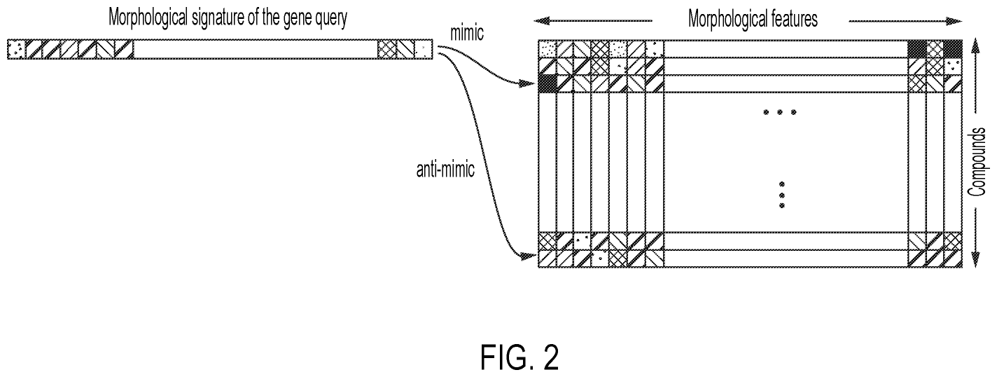

Cell Painting, as described in Example 1, was used to identify a morphological signature of YAP1 and TAZ overexpression in U2OS cells. Of note, the overexpression of NF-κB members gave the opposite visual phenotype, implying a negative regulation (anti-correlation). This negative regulatory relationship was confirmed using transcription experiments. FIG. 2 provides an example characterization of matching the morphological signature of a gene query to the signature(s) of compounds in a library. Connections which show both positive and negative large correlations are considered as matches. In general, U2OS and A549 cells have been most commonly used, however, the staining protocol has worked well for MCF-7, 3T3, HTB-9, HeLa, HepG2, HEKTE, SH-SY5Y, HUVEC, HMVEC, primary human fibroblasts, primary human hepatocyte/3T3-J2 fibroblast co-cultures, and many more.

Using the morphological signature found for YAP1 and TAZ, data mining was performed seeking compounds that effected the same (or opposite) morphology by computationally matching the morphological signature to a public database of small molecule signatures (see Caicedo et al., Nat. Meth. 2017 September; 14(9), 849-63, doi:10.1038/nMeth.4397, incorporated herein by reference). Specifically, the intersection of features in the two datasets was determined, resulting in 605 features (1399 features in the genetic screen, without any feature selection; and 729 features in the compound screen, obtained using the findCorrelation function with a threshold of 0.90 on the original 1,783 dimensional feature set). In order to make values of the corresponding features comparable, each feature was standardized with respect to the negative control. This standardization was done plate wise, based on the mean and standard deviation of the controls at profile level for the compound dataset. The normalization parameters were slightly different for the genetic screen, where median and median absolute deviation (MAD) were used instead, to remove the outlier effects (see Rohban et al., eLife 2017; 6:e24060). The signatures were then obtained by averaging the replicate profiles feature-wise. Pearson correlation was used on the aligned signatures of a gene and compound to score their connection.

From this exercise, 30 compounds (Table 1) were identified as potential regulators of these pathways (see Table 2). Correlation values for these compounds being either higher than 0.35 or lower than −0.35 indicate the compounds as candidate regulators of the Hippo and/or NF-κB pathways, based on the analysis of a benchmark subset of compounds that are bioactive and have annotated targets. Connections with an absolute correlation higher than 0.35 were 2.5 times more enriched in being correctly paired, compared to random connections (p-value=0.007).

TABLE 2
Results from Data Mining Using the YAP1/TAZ Morphological Signature

| Compound | | | |
|---|---|---|---|
| ID | Corr. to YAP1 cluster | Avg. Cell Count z-score | Corr. to TRAF2 |
| BRD- | −0.451816097371088 | −1.09876751317736 | 0.483378769177758 |
| K96698997 | | | |
| BRD- | −0.442645220271481 | 2.10395320180544 | 0.359126741534968 |
| K13719685 | | | |
| BRD- | −0.439820984888043 | 0.478037482862912 | 0.286882376707942 |
| K34692511 | | | |
| BRD- | −0.431674202259506 | −2.04414892904578 | 0.406783833287405 |
| K28862419 | | | |
| BRD- | −0.419476423486351 | 0.57275101988869 | 0.309863968937689 |
| K70003473 | | | |
| BRD- | 0.428418602602677 | −0.725175228242348 | −0.339745295430873 |
| K46678324 | | | |
| BRD- | 0.435680365509664 | −0.790948517843583 | −0.451237141338472 |
| K06593056 | | | |
| BRD- | 0.440145834465088 | 0.820309153702101 | −0.33133766243123 |
| A61154809 | | | |
| BRD- | 0.445765052349004 | −1.05491865344321 | −0.466745148730574 |
| K77793136 | | | |
| BRD- | 0.44641427678599 | −0.656771007057064 | −0.493557215874781 |
| K15567136 | | | |
| BRD- | 0.450273057324876 | −0.21652845532613 | −0.349609799358579 |
| K88429204 | | | |
| BRD- | 0.458309143762898 | −0.881277168895946 | −0.371958320674078 |
| K42095107 | | | |
| BRD- | 0.462214494435451 | −0.570827241978117 | −0.377843726025219 |
| K43796186 | | | |
| BRD- | 0.480168234528875 | −0.00429997421281192 | −0.416740972979351 |
| K37451830 | | | |
| BRD- | 0.483314867234996 | −1.39693975936963 | −0.481607208500513 |
| K03953354 | | | |
| BRD- | 0.483549276460743 | 0.218452233236704 | −0.428362405612274 |
| K39839146 | | | |
| BRD- | 0.488328497910833 | −0.739206863357278 | −0.52723612634195 |
| K62768599 | | | |
| BRD- | 0.488715954293843 | −0.937403709355667 | −0.272772324093652 |
| K42367391 | | | |
| BRD- | 0.490400094569485 | −1.70388177750873 | −0.362018046441038 |
| K22874335 | | | |
| BRD- | 0.491645117137496 | −0.0709502410087301 | −0.43890527237543 |
| K41723088 | | | |
| BRD- | 0.493953439051083 | −0.141108416583381 | −0.461903561022211 |
| K68530167 | | | |
| BRD- | 0.496607051682064 | −1.40921744009519 | −0.297027575600364 |
| K22754756 | | | |
| BRD- | 0.498791551452305 | 0.485053300420377 | −0.439765555665776 |
| K11266478 | | | |
| BRD- | 0.500357831891246 | 0.400863489730796 | −0.364867410280201 |
| K00135177 | | | |
| BRD- | 0.50094854898695 | −0.114799100742887 | −0.342751435013124 |
| K40143134 | | | |
| BRD- | 0.507801351616454 | −1.20225082214997 | −0.440615345966013 |
| K11758216 | | | |
| BRD- | 0.509868432726129 | 0.0980140318335538 | −0.413168132822434 |
| K48052543 | | | |
| BRD- | 0.51019424419505 | −0.118808139347153 | −0.475169203024327 |
| A50675702 | | | |
| BRD- | 0.526963070335337 | −0.553287698084454 | −0.406037778061057 |
| K28043081 | | | |
| BRD- | 0.548319649662317 | 0.77971763783391 | −0.466667305651416 |
| K19969618 | | | |

### Example 3: Proliferation Assay and Results

Proliferation assays were performed on the 30 compounds identified by the Data Mining described in Example 2. These studies identified three compounds of particular interest: BRD-K43796186 (Compound 13), BRD-K34692511 (Compound 3), and BRD-K28862419 (Compound 4).

Mouse sarcoma cells were treated with 1 μM of compound for 48 h. RNA was isolated and qRT-PCR was performed for known NF-κB targets and proliferation-associated targets (i.e., Ki67, FOXM1). Target genes used to assess NF-κB inhibition specific to the cancer contexts are evaluated. For example, to test the efficacy of these compounds against NF-κB transcriptional activity in ubiquitin-proteasome system cells, expression of PHLDA1, IER2, and LITAF is determined. Compounds that alter expression of NF-κB target genes are assessed in proliferation studies using UPS, head and neck, prostate, and breast cancer cell lines previously associated with NF-κB dependence. IC₅₀ studies and Western blot analyses are performed to evaluate total or phosphorylated p65 levels. Small molecules are then assessed to determine their mechanism of action.

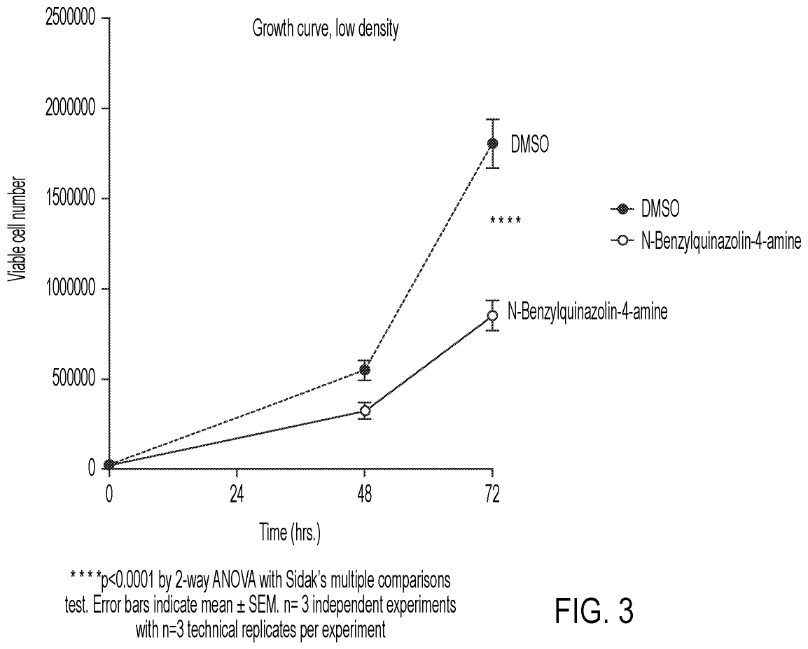

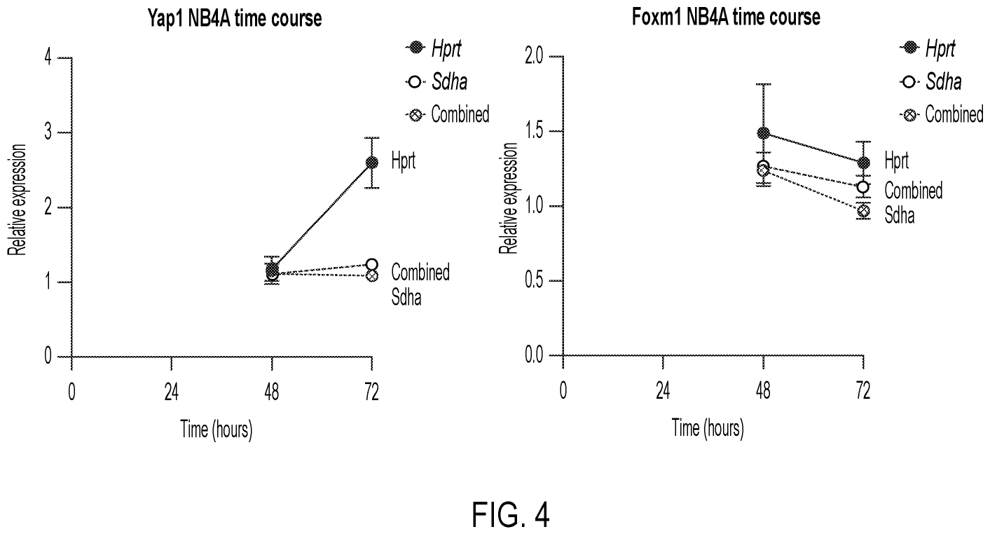

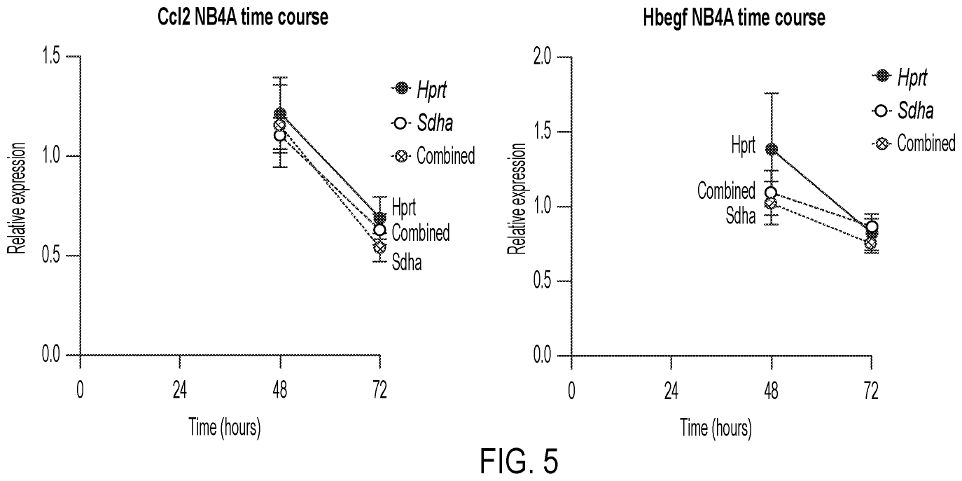

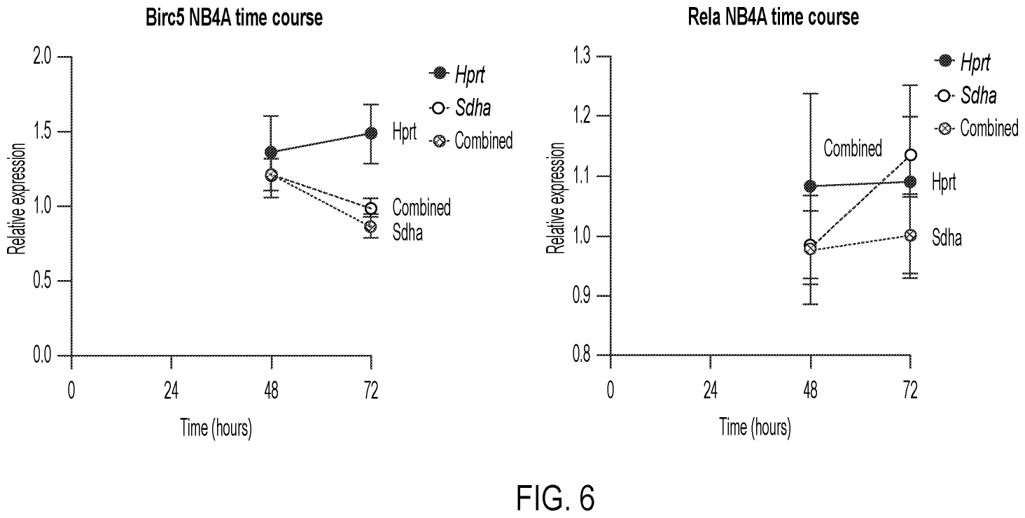

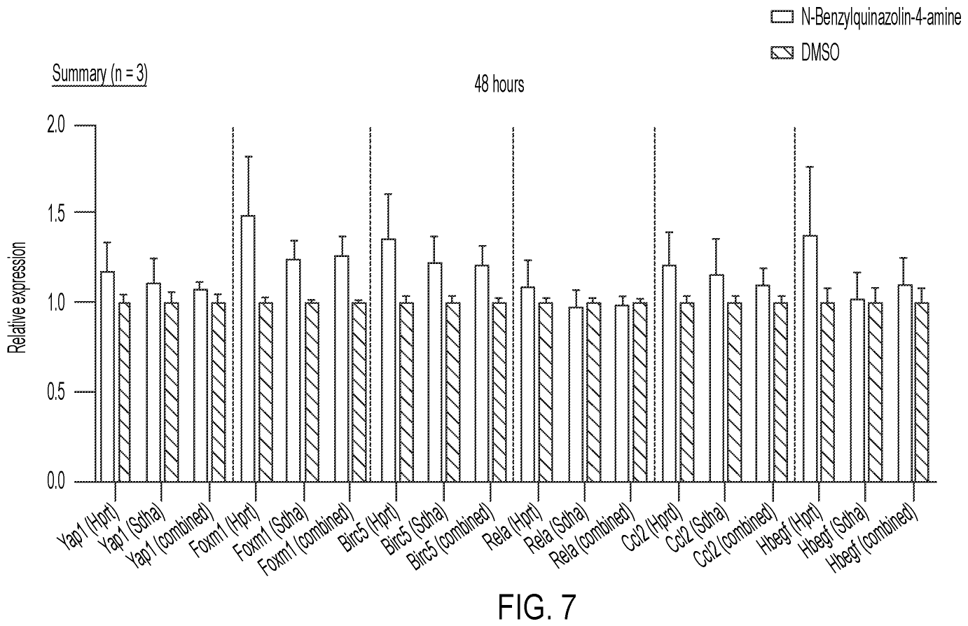

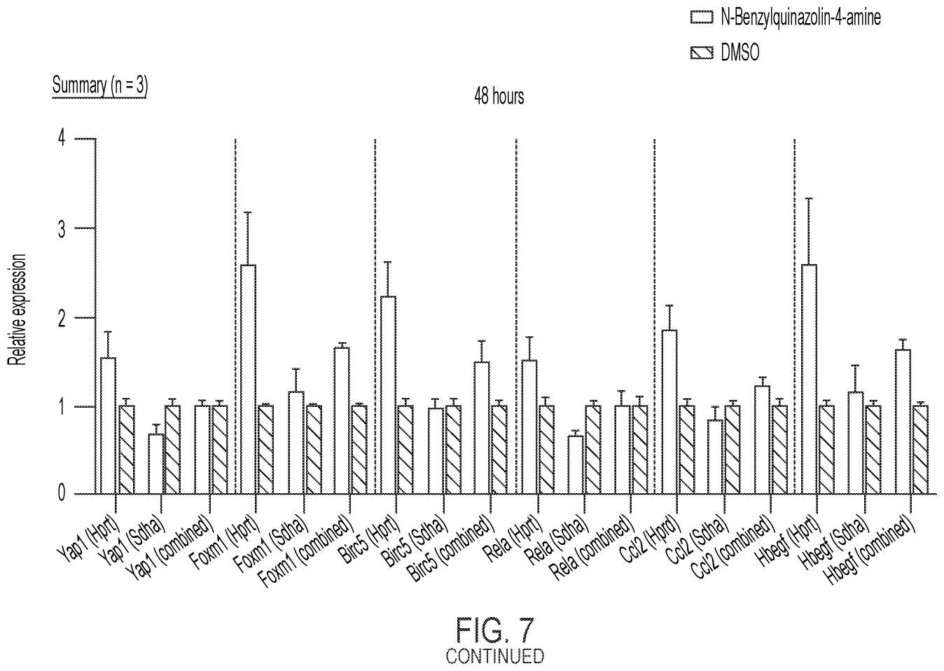

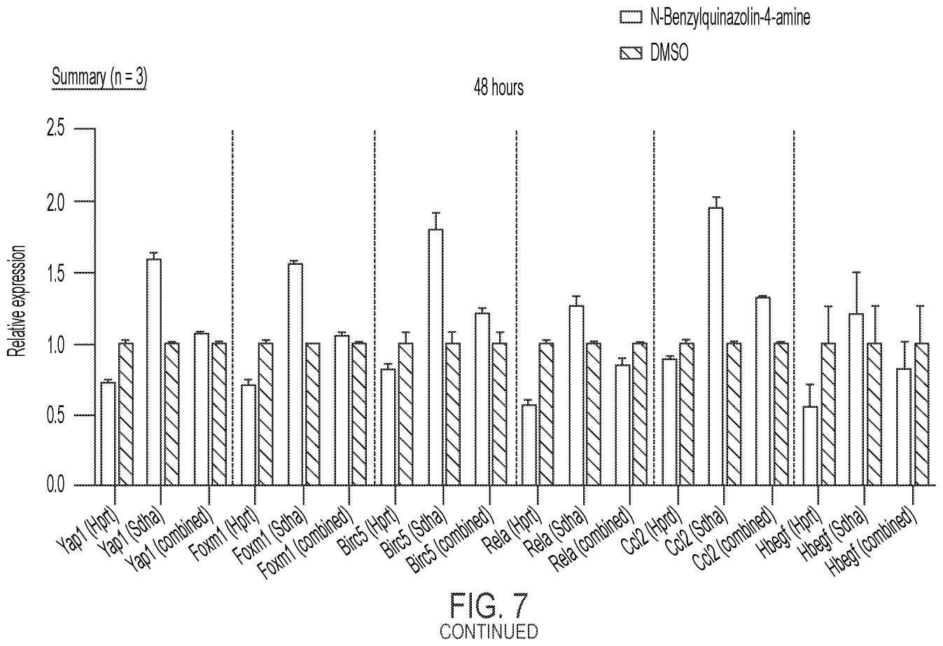

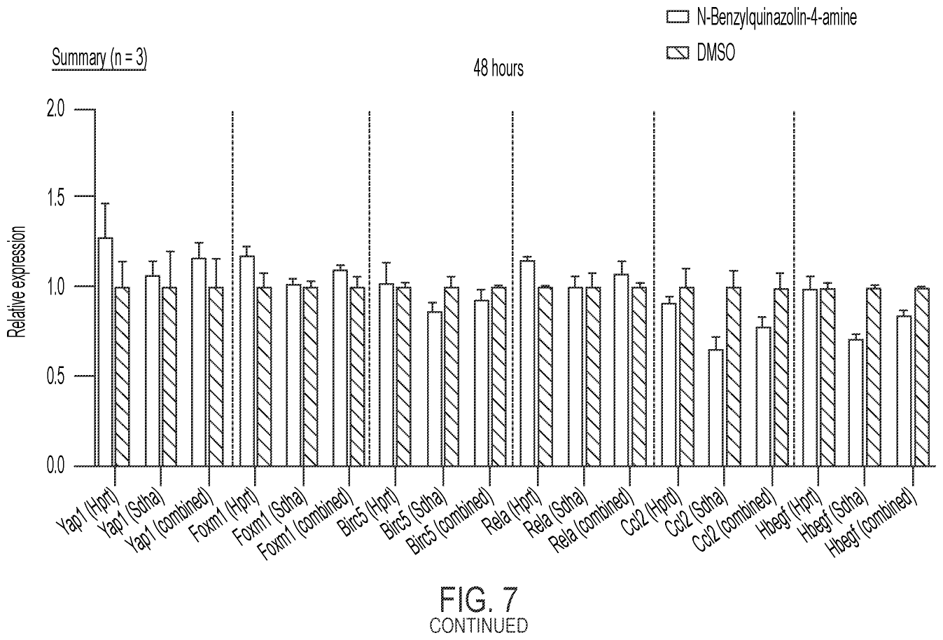

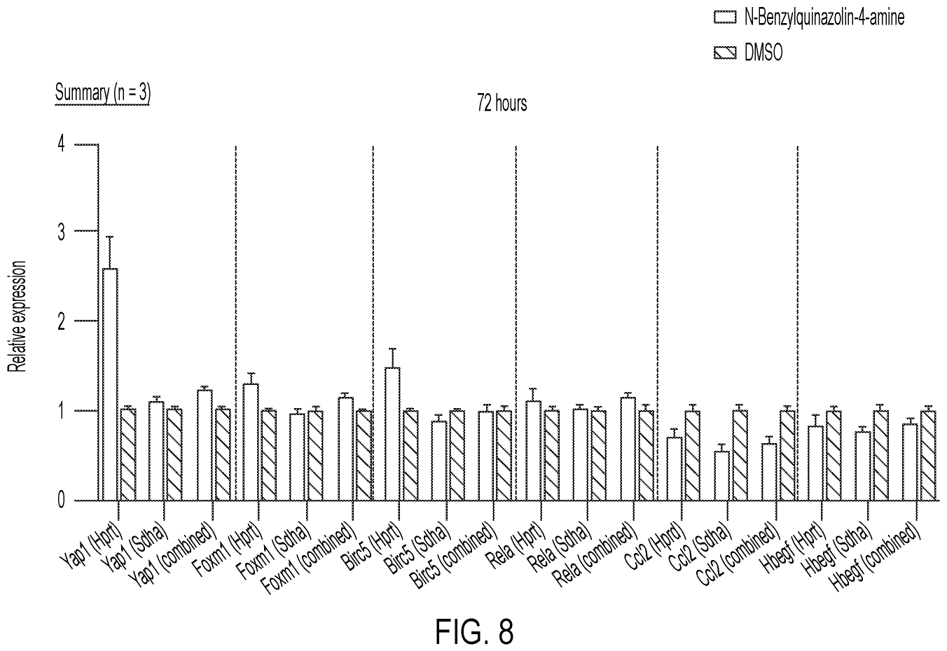

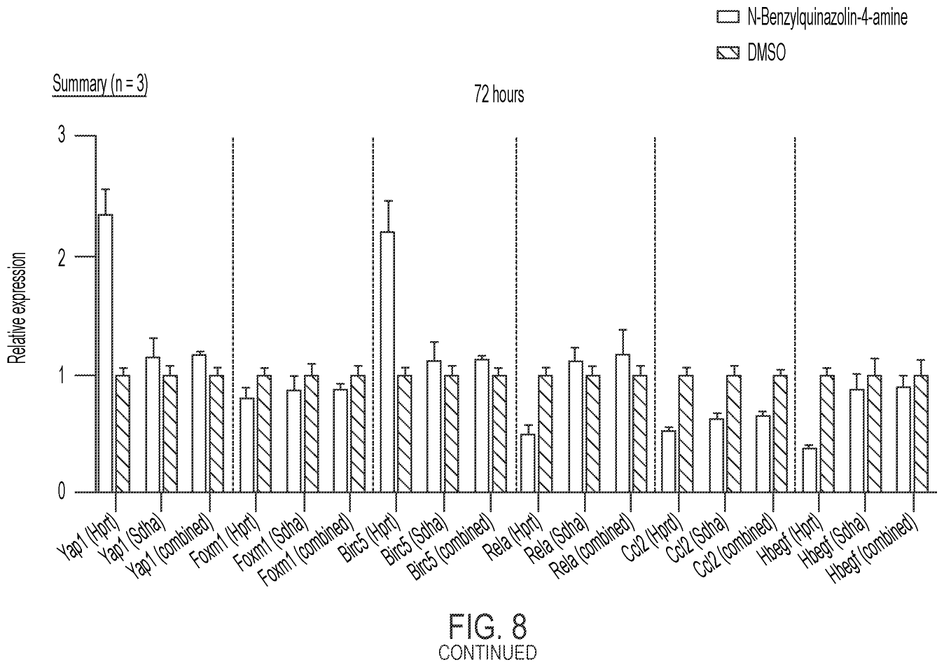

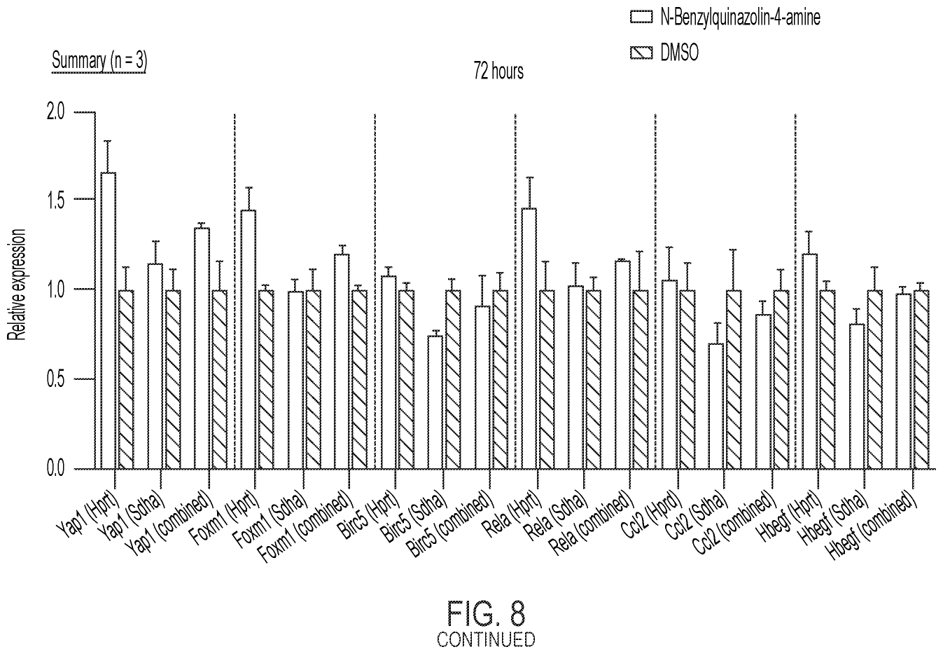

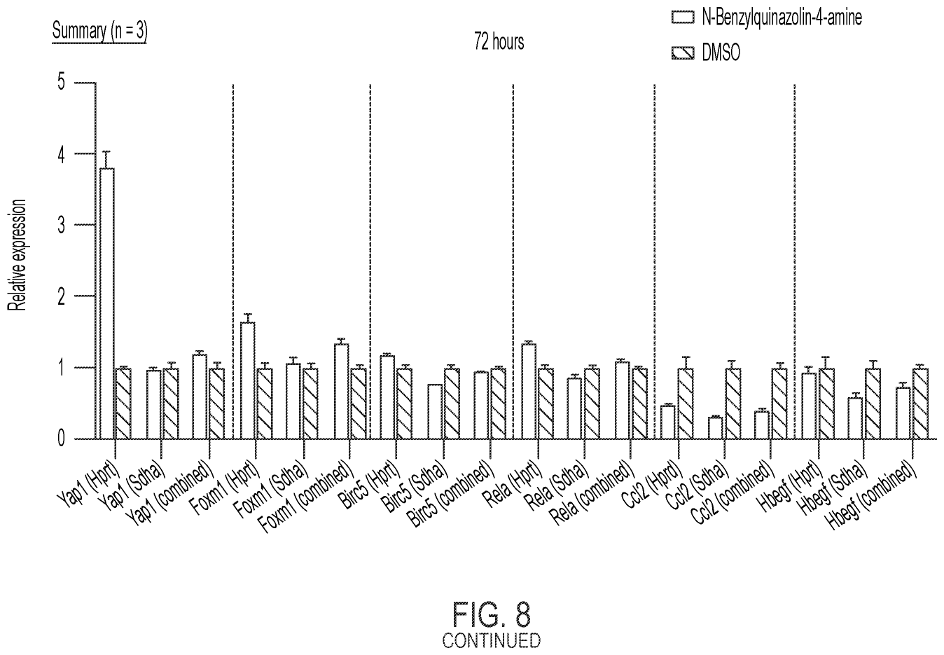

BRD-K43796186 (N-benzylquinazolin-4-amine) was then characterized in more detail. For example, FIG. 3 is a graph quantifying viable mouse sarcoma cell number vs. time for treatment with N-benzylquinazolin-4-amine (NB4A) and DMSO control (three independent experiments with three replicates per experiment). FIGS. 4-6 provide timecourse graphs showing the relative expression normalized to housekeeping genes. FIG. 4 provides two graphs showing the relative expression of Yap1, and the Yap1 target Foxm1 normalized to housekeeping genes Hprt, Sdha, or Hprt and Sdha combined in a N-benzylquinazolin-4-amine (NB4A) timecourse, showing that Yap1 gene expression is not effected, but Foxm1 expression is reduced. FIG. 5 provides two graphs showing the relative expression of Ccl2 and Hbegf normalized to housekeeping genes Hprt, Sdha, or Hprt and Sdha combined in a N-benzylquinazolin-4-amine (NB4A) timecourse, showing that when Ccl2 is normalized, expression is significantly decreased. FIG. 6 provides two graphs showing the relative expression of the Yap1 target Birc5 (left) and Rela, or p65, (right) normalized to housekeeping genes Hprt, Sdha, or Hprt and Sdha combined in a N-benzylquinazolin-4-amine (NB4A) timecourse. FIG. 7 is a set of four bar graphs showing relative expression of Yap1, Foxm1, Birc5, Rela, Ccl2, or Hbegf in the presence of N-benzylquinazolin-4-amine or a DMSO control for 48 hours, normalized to housekeeping genes Hprt, Sdha, or Hprt and Sdha combined. FIG. 8 is a set of four bar graphs showing relative expression of Yap1, Foxm1, Birc5, Rela, Ccl2, or Hbegf in the presence of N-benzylquinazolin-4-amine or a DMSO control for 72 hours, normalized to housekeeping genes Hprt, Sdha, or Hprt and Sdha.

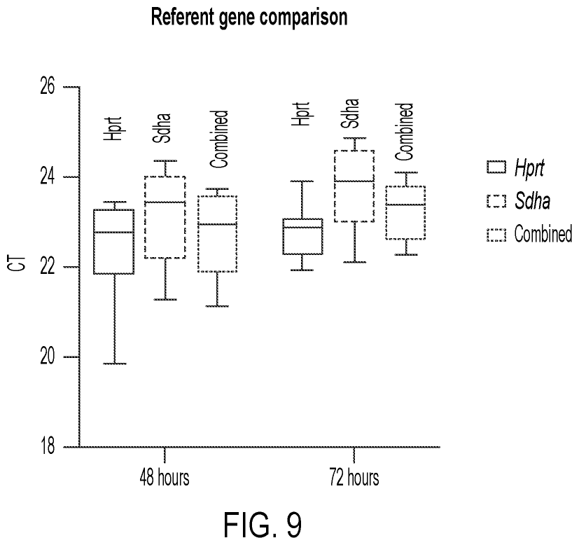

FIG. 9 is a graph of referent gene comparison displayed as CT vs. time. The genes are from left to right Hprt, Sdha, and Hprt and Sdha combined, at 48 h and 72 h. CT is the number of cycles needed for the fluorescence signal to reach a specific threshold level of detection which is inversely correlated with the amount of template nucleic acid present in the reaction.

In some embodiments, the effects of N-benzylquinazolin-4-amine and other compounds identified using Cell Painting and transcriptional analysis are tested in proliferation assays over longer time scales (e.g., days, weeks, months) and in different muscle-derived sarcoma cell lines with particular p53/Rb status. In some embodiments, the effects of the compounds at various dosages are assayed using Western blots to measure protein levels of YAP1 pathway targets, mRNA/cell count, and in cell culture, zebrafish, and mice to analyze migration and/or invasion and/or proliferation of tumor cells.

### Example 4: Discovery of Small Molecules Modulating the p38a (MAPK14) Pathway

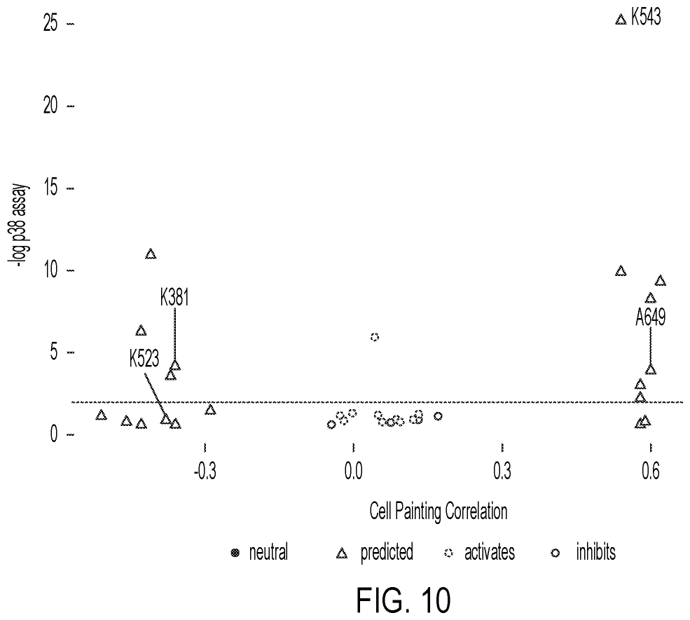

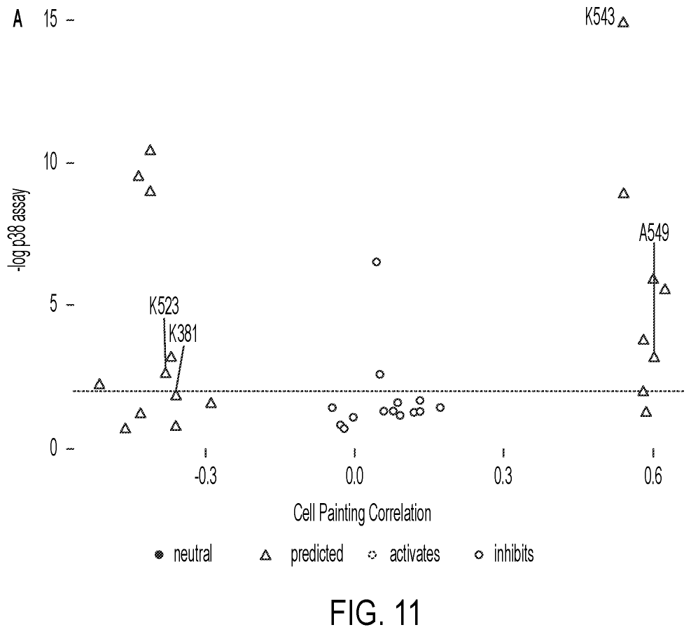

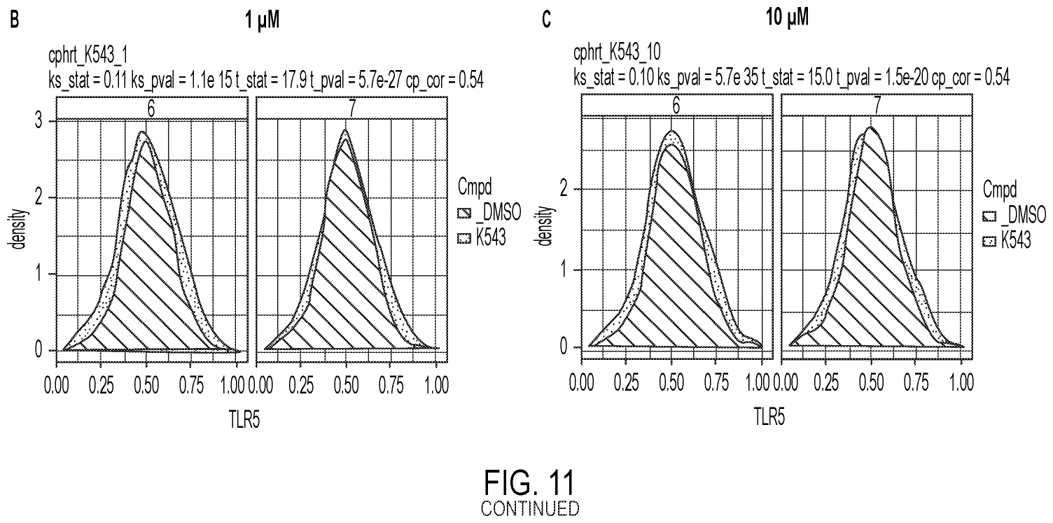

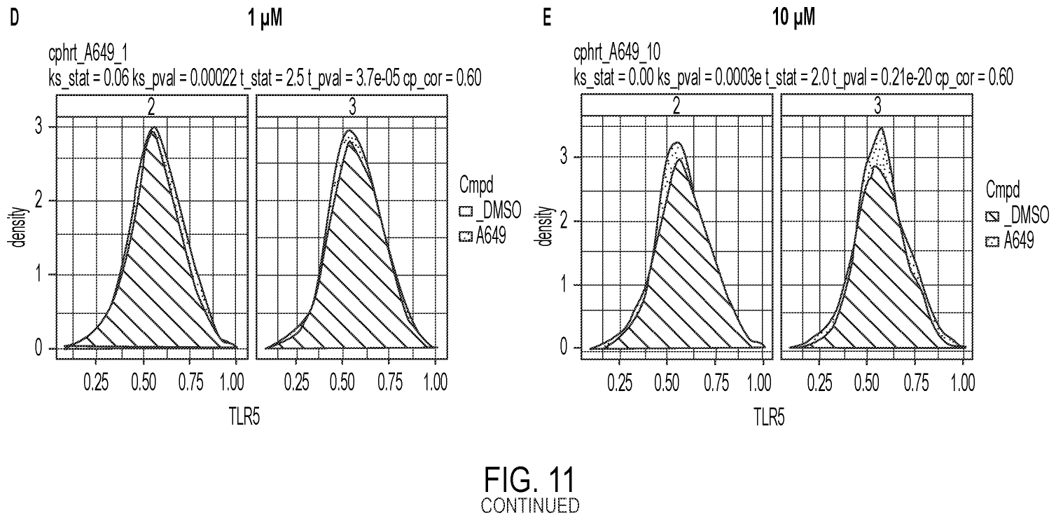

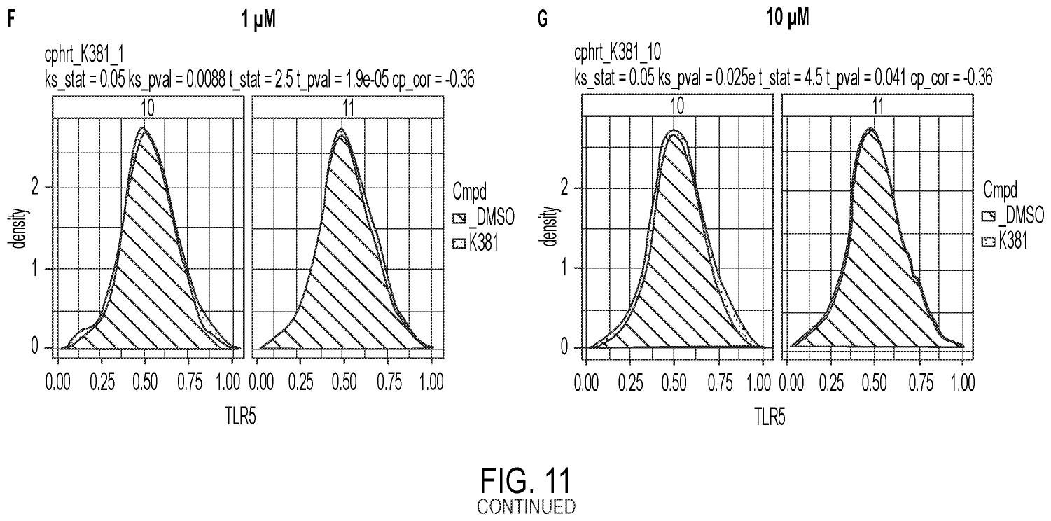

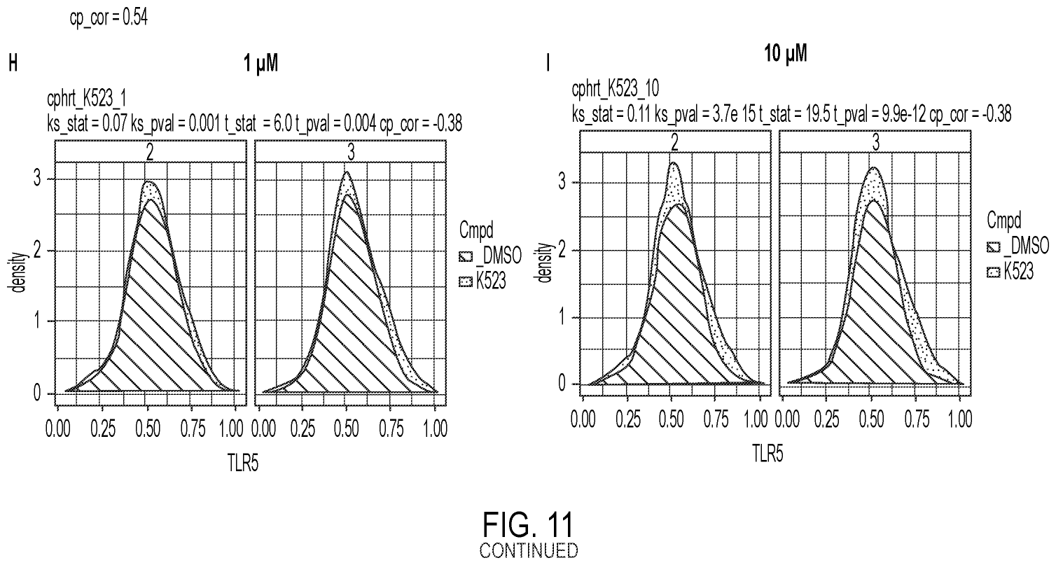

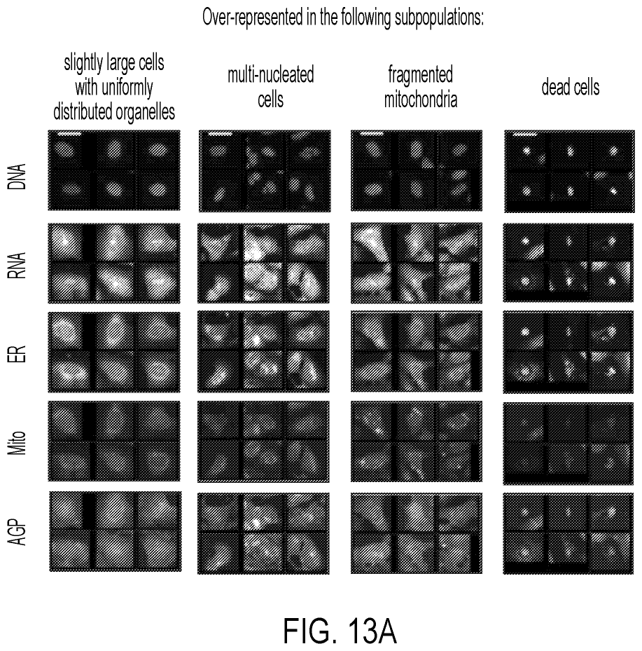

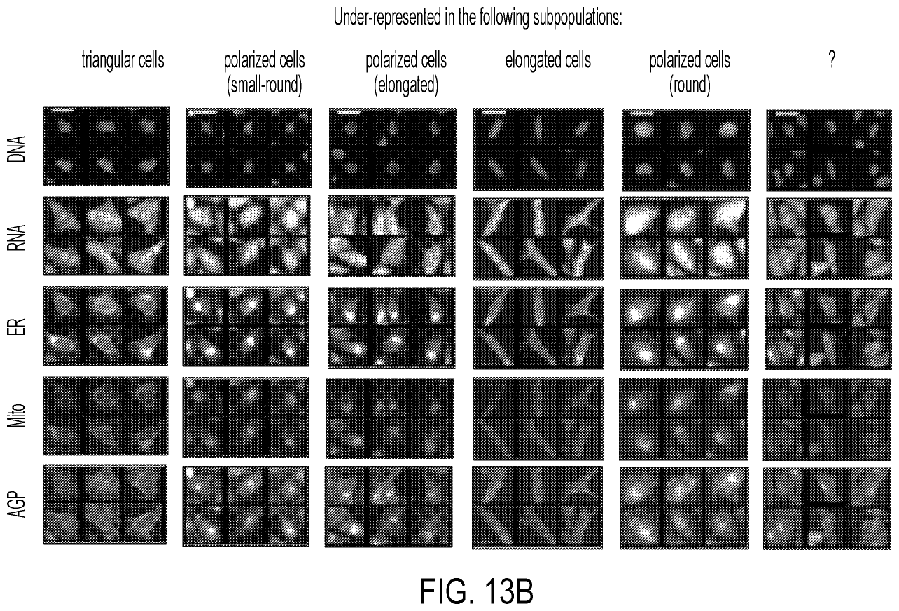

p38α (MAPK14) inhibitors are being sought for a wide variety of disorders, including various cancers, dementia, asthma, and COVID-19. We chose 20 compounds whose Cell Painting profile matched (9) or opposed (11) that of p38a overexpression in U2OS cells. The p38a pathway is activated by many stressors but rarely inhibited, so we focused on compounds that suppressed p38a activity. We found four such compounds in a single-cell p38a activity reporter assay in retinal pigment epithelial (RPE1) cells, including a known p38a MAPK inhibitor, SB202190 (4-(4-(4-fluorophenyl)-5-(pyridin-4-yl)-1H-imidazol-2-yl)phenol, FIG. 10) and confirmed activity at 10 μM as shown in FIG. 11. Therefore, our computational image-based matching method can identify novel compounds impacting the p38a pathway using public Cell Painting data rather than a specific screen designed to measure p38a activity. Two potential inhibitors were found (BRD-K38197229, 3-(butylamino)-4-phenoxy-5-sulfamoylbenzoic acid, referred to in the figure as <K381>) and BRD-A64933752, 2-(6-amino-2-anilinopurin-9-yl)-5-(hydroxymethyl)oxolane-3,4-diol, referred to in the figure as <A649>); an additional compound (BRD-K52394958, 5-Fluoro-3-[2-[4-methoxy-4-[[(R)-phenylsulfinyl]methyl]piperidin-1-yl]ethyl]-1H-indole, referred to in the figure as <523> was also identified via an alternative statistical test as shown in FIG. 13. BRD-54330070 (4-(4-Fluorophenyl)-2-(4-hydroxyphenyl)-5-(4-pyridyl)imidazole), SB202190), referred to in the figure as K543 is a known p38 inhibitor also identified as a match.

### Example 5: Discovery of Small Molecules Impacting PPARGC1A (PGC-1a) Overexpression Phenotypes

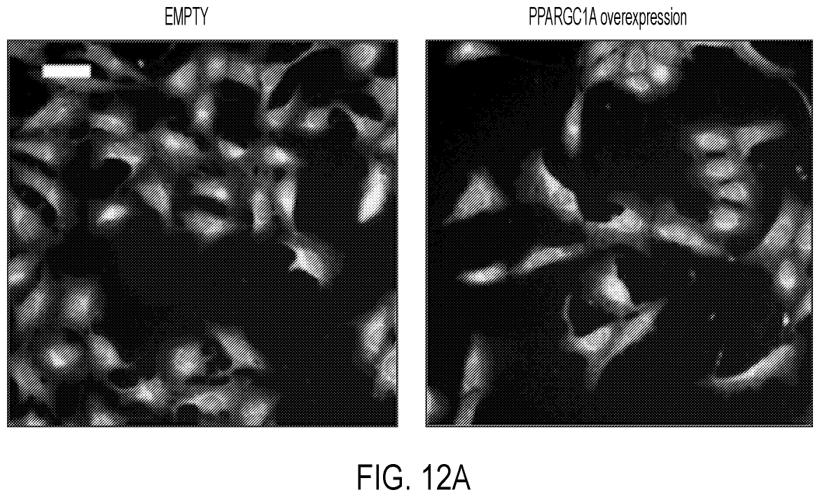

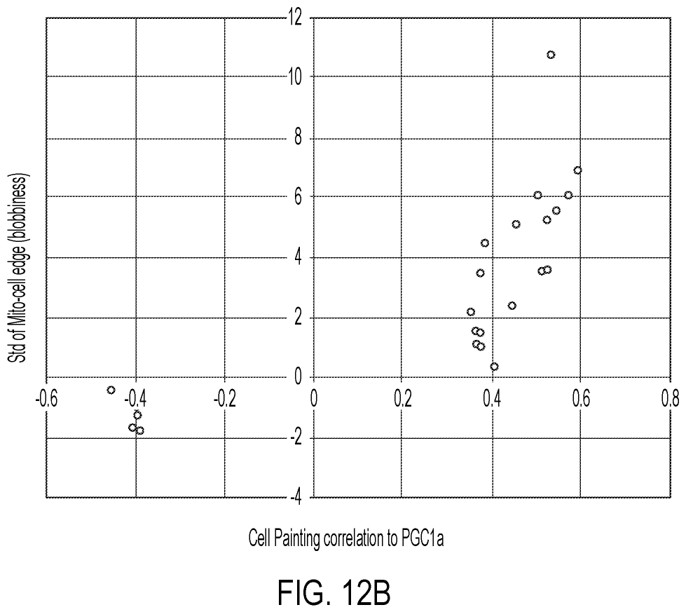

We next identified compounds with strong morphological correlation to overexpression of peroxisome proliferator-activated receptor gamma coactivator 1-alpha (PGC-1α, encoded by the PPARGC1A gene). These compounds generally followed the hits in a published, targeted screen for PGC-1α activity (p=7.7e-06, Fisher's exact test) as described in PubChem Bioassay Record AID 651723 (https://pubchem.ncbi.nlm.nih.gov/bioassay/651723), which is hereby incorporated by reference in its entirety. Identification of similar compounds validates the image profile-based matching approach described herein. The dominant matching phenotype is mitochondrial blobbiness, which can be quantified as the standard deviation of the MitoTracker staining at the edge of the cell being high without major changes to cell proliferation, size, or overall protein content (FIGS. 12A and 12B). Cell subpopulations that are large, multi-nucleate, and contain fragmented mitochondria are enriched when PGC-1α is overexpressed while subpopulations whose organelles are asymmetric are under-represented (FIG. 13). More symmetric organelle morphology is associated with reduced motility and PGC-1α overexpression. The role of PGC-1α in mitochondrial biogenesis is well-appreciated. The phenotype uncovered here using image profile matching is consistent with other recently discovered mitochondrial phenotypes associated with this gene such as the phenotypes described in Halling, J. F. et al. Appl. Physiol, Nutr. Metab. 45 (2020): 927-936, which is hereby incorporated by reference in its entirety.

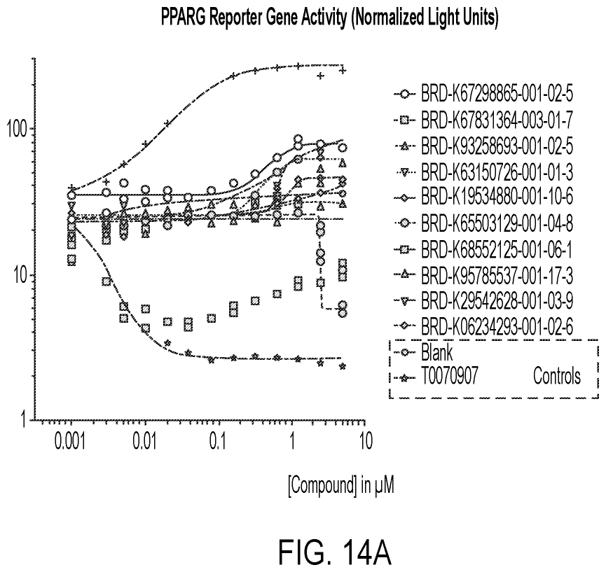

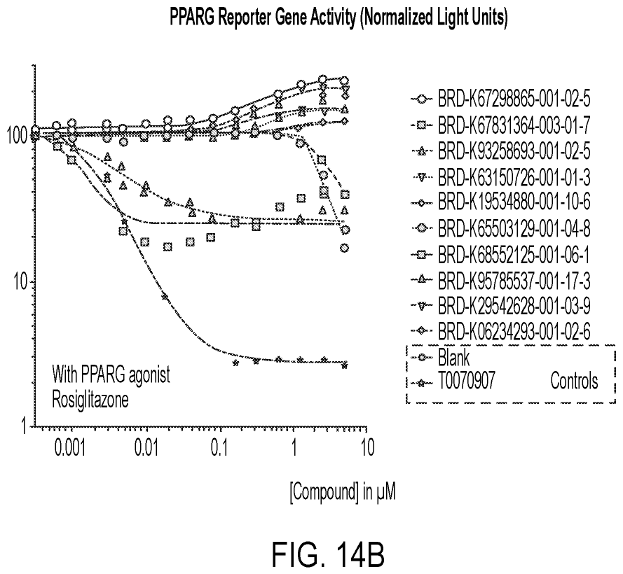

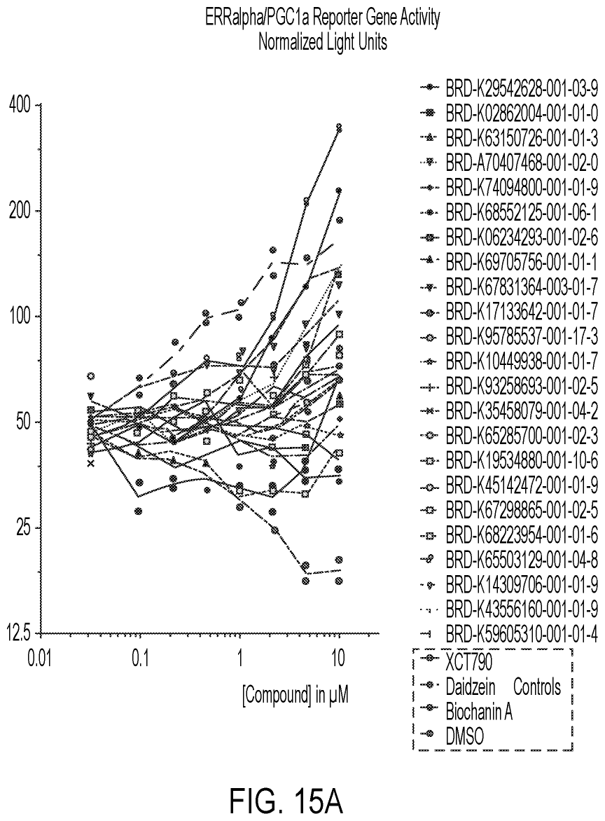

24 compounds were chosen with Cell Painting profiles which correlate or anti-correlate with PGC-1α overexpression in U2OS cells. One of these compounds is a known direct ligand for PPAR gamma, GW-9662 (BRD-K9325869, 2-Chloro-5-nitro-N-phenylbenzamide). PGC-1a is a transcriptional coactivator of several nuclear receptors including PPAR gamma and ERR alpha. We therefore tested compounds in a reporter assay representing FABP4, a prototypical target gene of the nuclear receptor, PPARG, in a bladder cancer cell line (FIGS. 14A and 14B). Three of the five most active compounds leading to reporter activation were structurally related and included two annotated SRC inhibitors, PP1 and PP2, which have a known link to PGC-1a, as well as a novel analog thereof. CCT018159 (BRD-K65503129) and Phorbol 12-myristate 13-acetate (BRD-K68552125) inhibited reporter activity. Compounds identified in FIG. 14 are provided in Table 3.

TABLE 3

| Compound ID | Structure | Name |
|---|---|---|
| BRD- K67298865 | 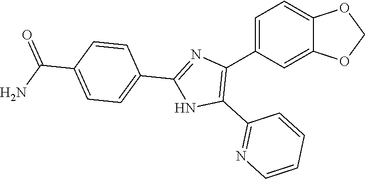 [SMILES: NC(=O)c1ccc(-c2nc(-c3ccc4c(c3)OCO4)c(-c3ccccn3)[nH]2)cc1] | 4-(4-(benzo[d][1,3]dioxol- 5-yl)-5-(pyridin-2-yl)-1H- imidazol-2-yl)benzamide |
| BRD- K67831364 | 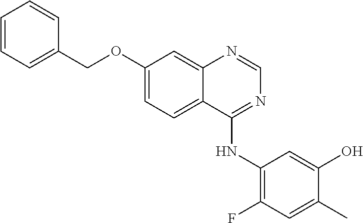 [SMILES: Cc1cc(F)c(Nc2ncnc3cc(OCc4ccccc4)ccc23)cc1O] | 5-((7- (benzyloxy)quinazolin-4- yl)amino)-4-fluoro-2- methylphenol |
| BRD- K93258693 | 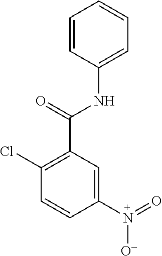 | 2-chloro-5-nitro-N- phenylbenzamide |
| BRD- K63150726 | 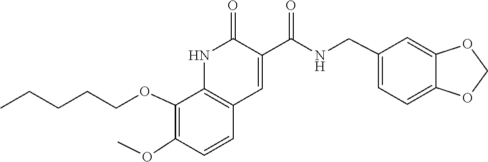 [SMILES: CCCCCOc1c(OC)ccc2cc(C(=O)NCc3ccc4c(c3)OCO4)c(=O)[nH]c12] | n-(1,3-benzodioxol-5- ylmethyl)-1,2-dihydro-7- methoxy-2-oxo-8- (pentyloxy)-3- quinolinecarboxamide |
| BRD- K19534880 | 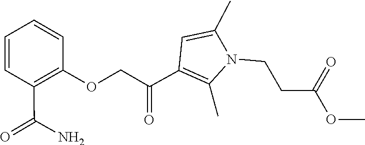 [SMILES: COC(=O)CCn1c(C)cc(C(=O)COc2ccccc2C(N)=O)c1C] | methyl 3-(3-(2-(2- carbamoylphenoxy)acetyl)- 2,5-dimethyl-1H-pyrrol-1- yl)propanoate |
| BRD- K65503129 | 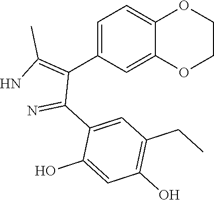 [SMILES: CCc1cc(C(=N)/C(=C(/C)N)c2ccc3c(c2)OCCO3)c(O)cc1O] | 4-[4-(2,3-dihydro-1,4- benzodioxin-6-yl)-5- methyl-1H-pyrazol-3-yl]- 6-ethylbenzene-1,3-diol |
| BRD- K68552125 | 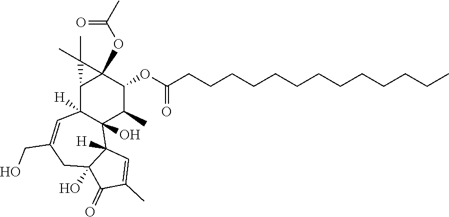 | 12-O- Tetradecanoylphorbol-13- acetate |
| BRD- K95785537 | 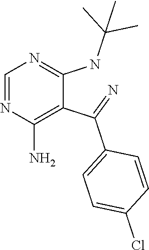 [SMILES: CC(C)(C)Nc1ncnc(N)c1C(=N)c1ccc(Cl)cc1] | 1-(tert-butyl)-3-(4- chlorophenyl)-1H- pyrazolo[3,4-d]pyrimidin- 4-amine |
| BRD- K29542628 | 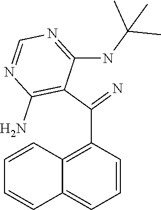 [SMILES: CC(C)(C)Nc1ncnc(N)c1C(=N)c1cccc2ccccc12] | 1-(tert-Butyl)-3- (naphthalen-1-yl)-1H- pyrazolo[3,4-d]pyrimidin- 4-amine |
| BRD- K06234293 | 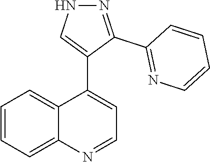 [SMILES: c1ccc(-c2n[nH]cc2-c2ccnc3ccccc23)nc1] | 4-(3-(pyridin-2-yl)-1H- pyrazol-4-yl)quinoline |
| T0070907 | 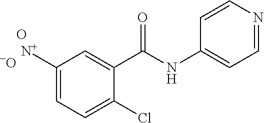 | 2-chloro-5-nitro-N- (pyridin-4-yl)benzamide |

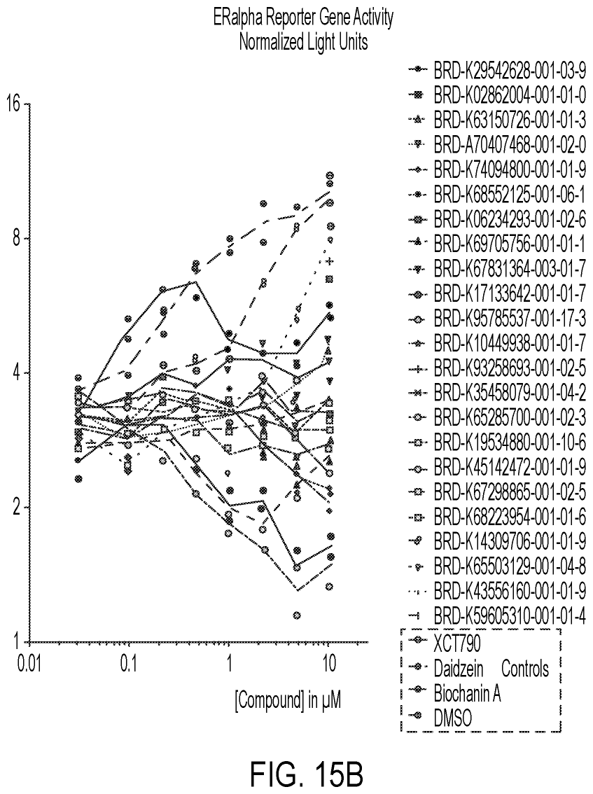

Additionally, many of the same compounds also showed activity in an ERRalpha reporter assay in 293T cells, albeit with opposing effects (FIGS. 15A and 151B). Compounds identified in FIGS. 15A and 15B are provided in Table 4.

TABLE 4

| Compound ID | Structure | Name |
|---|---|---|
| BRD- K29542628 | 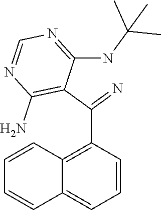 [SMILES: CC(C)(C)Nc1ncnc(N)c1C(=N)c1cccc2ccccc12] | 1-tert-butyl-3-naphthalen-1- ylpyrazolo[3,4-d]pyrimidin- 4-amine |
| BRD- K02862004 | 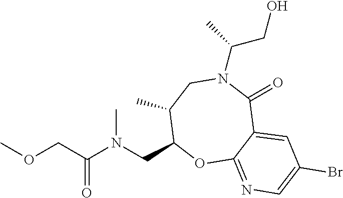 | N-[[(2R,3R)-8-bromo-5- [(2R)-1-hydroxypropan-2- yl]-3-methyl-6-oxo-3,4- dihydro-2H-pyrido[2,3- b][1,5]oxazocin-2- yl]methyl]-2-methoxy-N- methylacetamide |
| BRD- K63150726 | 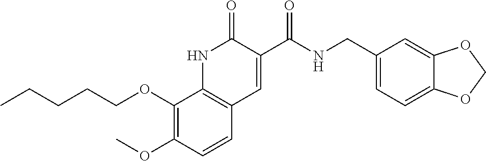 [SMILES: CCCCCOc1c(OC)ccc2cc(C(=O)NCc3ccc4c(c3)OCO4)c(=O)[nH]c12] | N-(1,3-benzodioxol-5- ylmethyl)-7-methoxy-2-oxo- 8-pentoxy-1H-quinoline-3- carboxamide |
| BRD- A70407468 | 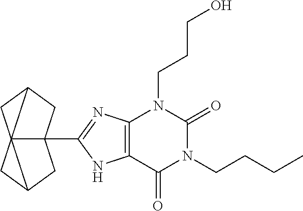 | 1-butyl-3-(3-hydroxypropyl)- 8-(3- tricyclo[3.3.1.0^{3,7}]nonanyl)- 7H-purine-2,6-dione |
| BRD- K74094800 | 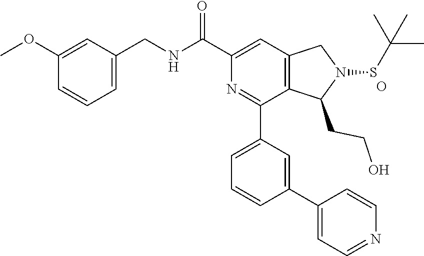 | (3S)-2-[(S)-tert- butylsulfinyl]-3-(2- hydroxyethyl)-N-[(3- methoxyphenyl)methyl]-4- (3-pyridin-4-ylphenyl)-1,3- dihydropyrrolo[3,4- c]pyridine-6-carboxamide |
| BRD- K68552125 | 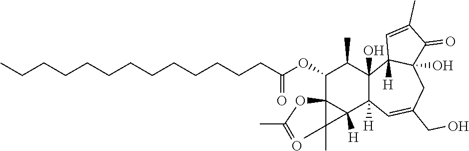 | [(1S,2S,6R,10S,11R,13S,14R, 15R)-13-acetyloxy-1,6- dihydroxy-8- (hydroxymethyl)-4,12,12,15- tetramethyl-5-oxo-14- tetracyclo[8.5.0.0^{2,6}.0^{11,13}] pentadeca-3,8-dienyl] tetradecanoate |
| BRD- K06234293 | 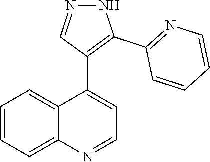 [SMILES: c1ccc(-c2[nH]ncc2-c2ccnc3ccccc23)nc1] | 4-(5-pyridin-2-yl-1H- pyrazol-4-yl)quinoline |
| BRD- K69705756 | 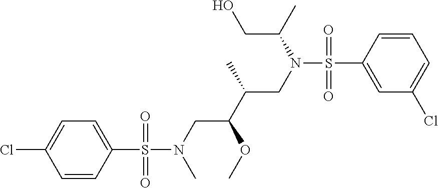 [SMILES: CO[C@@H](CN(C)S(=O)(=O)c1ccc(Cl)cc1)[C@H](C)CN([C@@H](C)CO)S(=O)(=O)c1cccc(Cl)c1] | 3-chloro-N-((2R,3R)-4-((4- chloro-N- methylphenyl)sulfonamido)- 3-methoxy-2-methylbutyl)- N-((S)-1-hydroxypropan-2- yl)benzenesulfonamide |
| BRD- K67831364 | 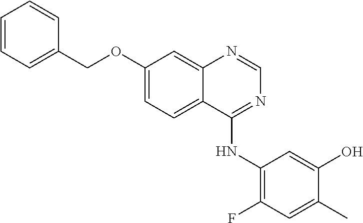 [SMILES: Cc1cc(F)c(Nc2ncnc3cc(OCc4ccccc4)ccc23)cc1O] | 5-((7-(benzyloxy)quinazolin- 4-yl)amino)-4-fluoro-2- methylphenol |
| BRD- K17133642 | 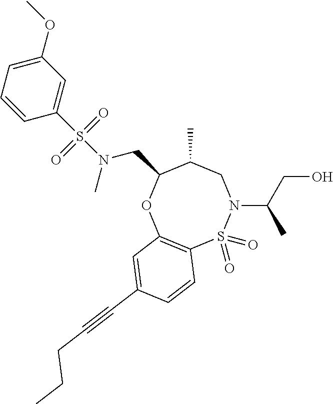 [SMILES: CCCC#Cc1ccc2c(c1)O[C@@H](CN(C)S(=O)(=O)c1cccc(OC)c1)[C@H](C)CN([C@H](C)CO)S2(=O)=O] | N-(((4R,5R)-2-((R)-1- hydroxypropan-2-yl)-4- methyl-1,1-dioxido-8-(pent- 1-yn-1-yl)-2,3,4,5- tetrahydrobenzo[b][1,4,5] oxathiazocin-5-yl)methyl)-3- methoxy-N- methylbenzenesulfonamide |
| BRD- K95785537 | 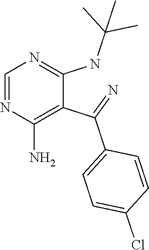 [SMILES: CC(C)(C)Nc1ncnc(N)c1C(=N)c1ccc(Cl)cc1] | 1-tert-butyl-3-(4- chlorophenyl)pyrazolo[3,4- d]pyrimidin-4-amine |
| BRD- K10449938 | 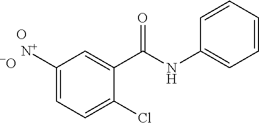 | 3-chloro-N-[(2R,3R)-4-[(4- chlorophenyl)sulfonyl- methylamino]-3-methoxy-2- methylbutyl]-N-[(2R)-1- hydroxypropan-2- yl]benzenesulfonamide |
| BRD- K93258693 | 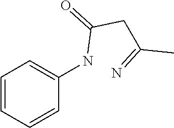 | 2-chloro-5-nitro-N- phenylbenzamide |
| BRD- K35458079 | 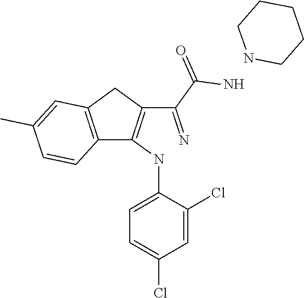 | 5-methyl-2-phenyl-4H- pyrazol-3-one |
| BRD- K65285700 | 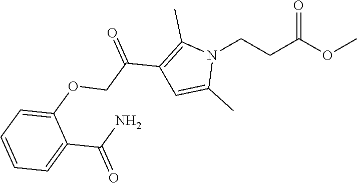 [SMILES: COC(=O)CCn1c(C)cc(C(=O)COc2ccccc2C(N)=O)c1C] | 1-(2,4-dichlorophenyl)-6- methyl-N-piperidin-1-yl-4H- indeno[1,2-c]pyrazole-3- carboxamide |
| BRD- K19534880 | 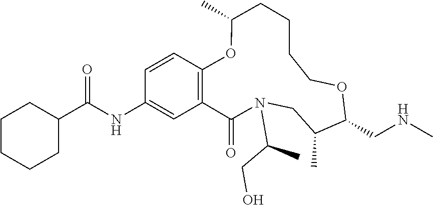 [SMILES: CNC[C@H]1OCCCC[C@@H](C)Oc2ccc(NC(=O)C3CCCCC3)cc2C(=O)N([C@@H](C)CO)C[C@H]1C] | methyl 3-[3-[2-(2- carbamoylphenoxy)acetyl]- 2,5-dimethylpyrrol-1- yl]propanoate |
| BRD- K45142472 | 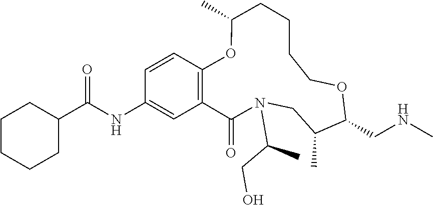 [SMILES: CNC[C@H]1OCCCC[C@@H](C)Oc2ccc(NC(=O)C3CCCCC3)cc2C(=O)N([C@@H](C)CO)C[C@H]1C] | N-[(3R,9S,10R)-12-[(2S)-1- hydroxypropan-2-yl]-3,10- dimethyl-9- (methylaminomethyl)-13- oxo-2,8-dioxa-12- azabicyclo[12.4.0]octadeca- 1(14),15,17-trien-16- yl]cyclohexanecarboxamide |
| BRD- K67298865 | 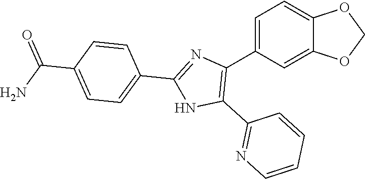 [SMILES: NC(=O)c1ccc(-c2nc(-c3ccc4c(c3)OCO4)c(-c3ccccn3)[nH]2)cc1] | 4-[4-(1,3-benzodioxol-5-yl)- 5-pyridin-2-yl-1H-imidazol- 2-yl]benzamide |
| BRD- K68223954 | 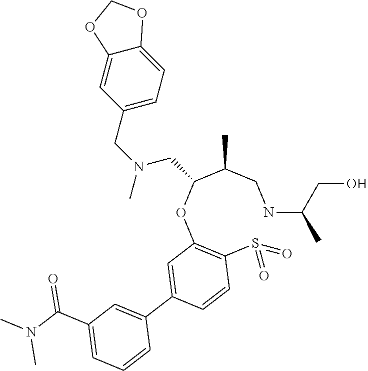 | 3-((4S,5S)-5- (((benzo[d][1,3]dioxol-5- ylmethyl)(methyl)amino) methyl)-2-((R)-1- hydroxypropan-2-yl)-4- methyl-1,1-dioxido-2,3,4,5- tetrahydrobenzo[b][1,4,5] oxathiazocin-8-yl)-N,N- dimethylbenzamide |
| BRD- K14309706 | 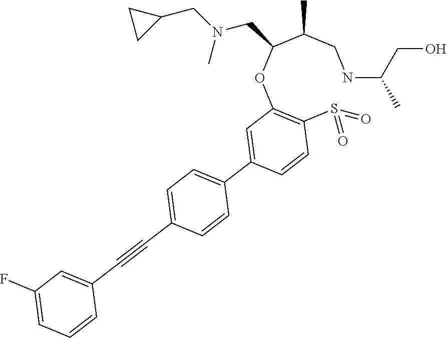 | (4S,5R)-5- (((cyclopropylmethyl)(methyl) amino)methyl)-8-(4-((3- fluorophenyl) ethynyl)phenyl)-2-((S)-1- hydroxypropan-2-yl)-4- methyl-2,3,4,5- tetrahydrobenzo[b][1,4,5] oxathiazocine 1,1-dioxide |
| BRD- K65503129 | 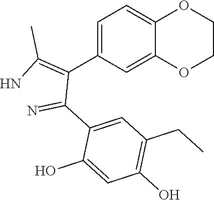 [SMILES: CCc1cc(C(=N)/C(=C(/C)N)c2ccc3c(c2)OCCO3)c(O)cc1O] | 4-[4-(2,3-dihydro-1,4- benzodioxin-6-yl)-5-methyl- 1H-pyrazol-3-yl]-6- ethylbenzene-1,3-diol |
| BRD- K43556160 | 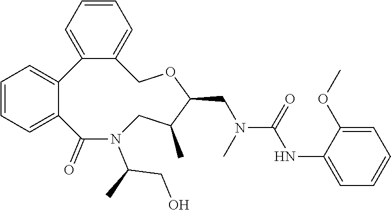 | 1-[[(10R,11S)-13-[(2R)-1- hydroxypropan-2-yl]-11- methyl-14-oxo-9-oxa-13- azatricyclo[13.4.0.0^{2,7}] nonadeca-1(19),2,4,6,15,17- hexaen-10-yl]methyl]-3-(2- methoxyphenyl)-1- methylurea |
| BRD- K43556160 | 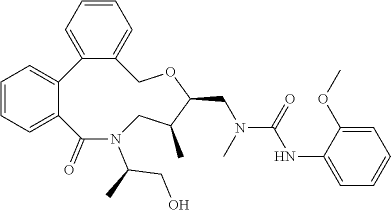 | 1-[[(10R,11S)-13-[(2R)-1- hydroxypropan-2-yl]-11- methyl-14-oxo-9-oxa-13- azatricyclo[13.4.0.0^{2,7}] nonadeca-1(19),2,4,6,15,17- hexaen-10-yl]methyl]-3-(2- methoxyphenyl)-1- methylurea |
| BRD- K59605310 | 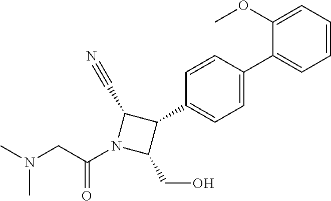 [SMILES: COc1ccccc1-c1ccc([C@H]2[C@@H](C#N)N(C(=O)CN(C)C)[C@H]2CO)cc1] | (2S,3S,4R)-1-[2- (dimethylamino)acetyl]-4- (hydroxymethyl)-3-[4-(2- methoxyphenyl)phenyl] azetidine-2-carbonitrile |

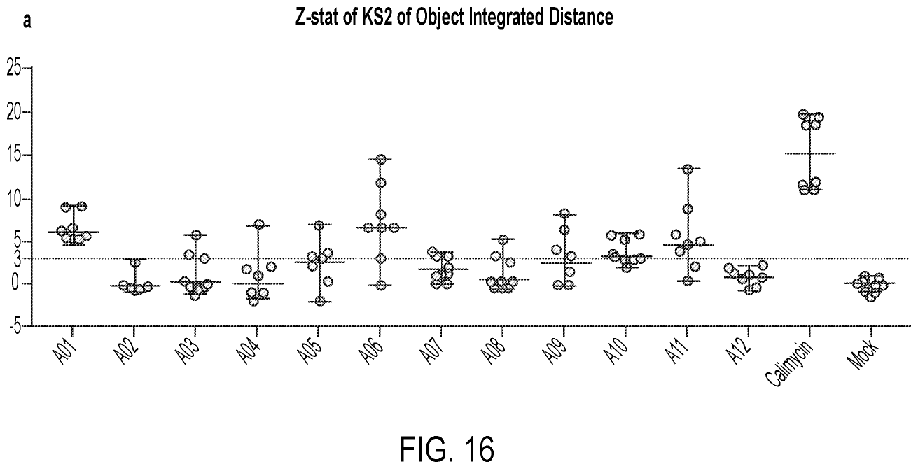

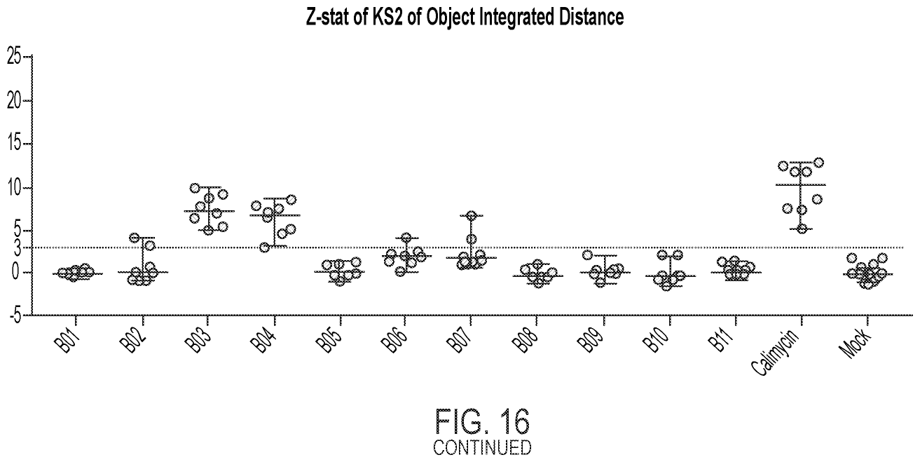

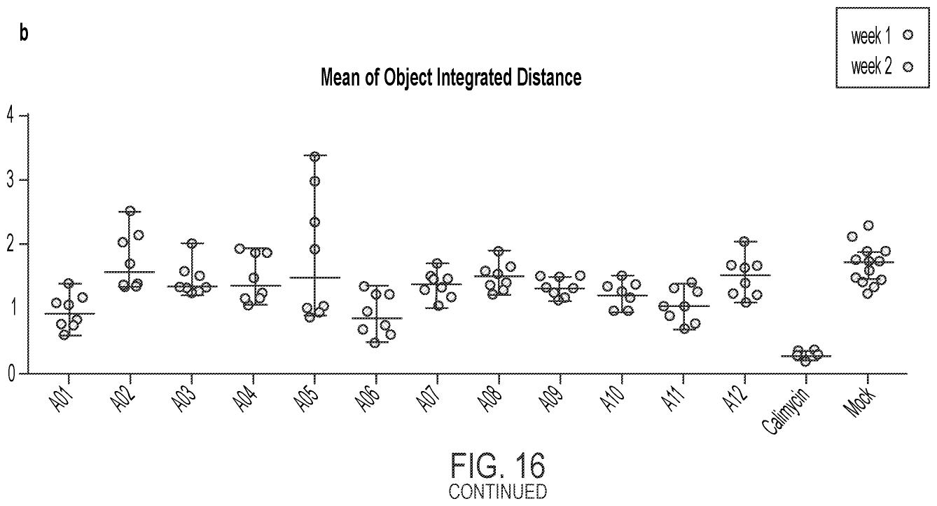

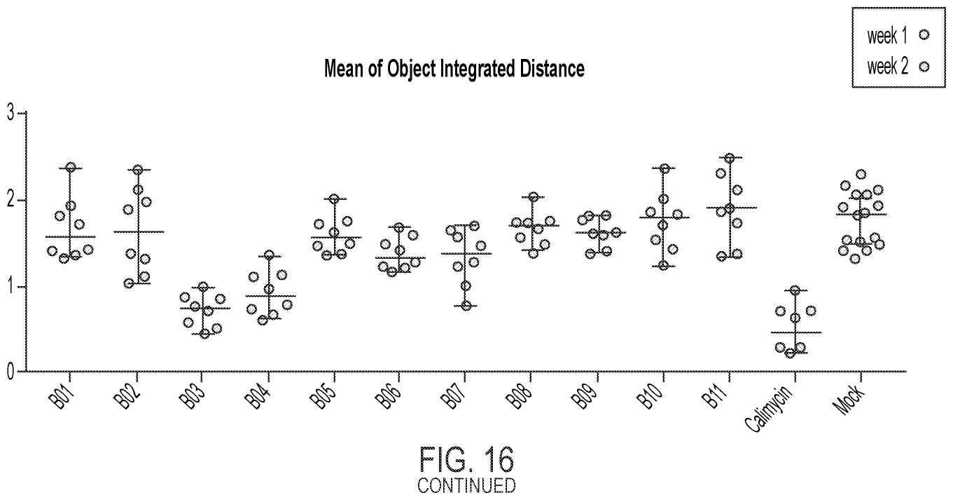

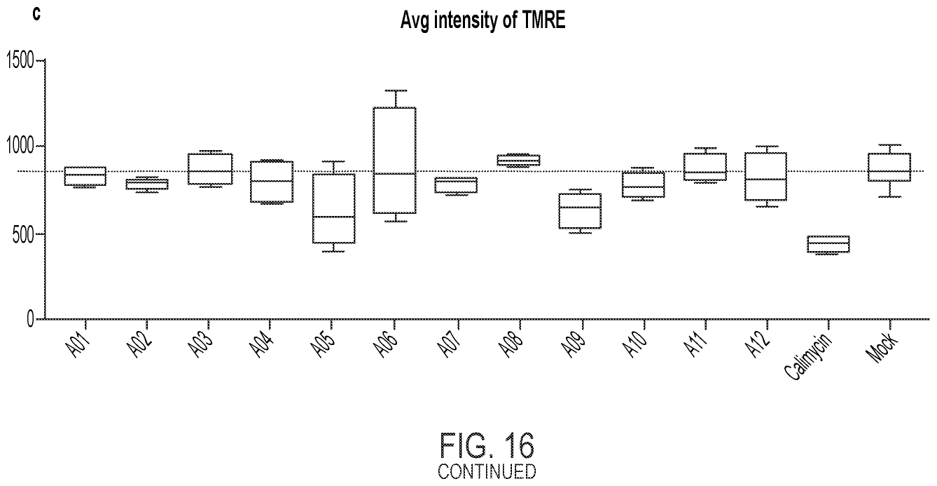

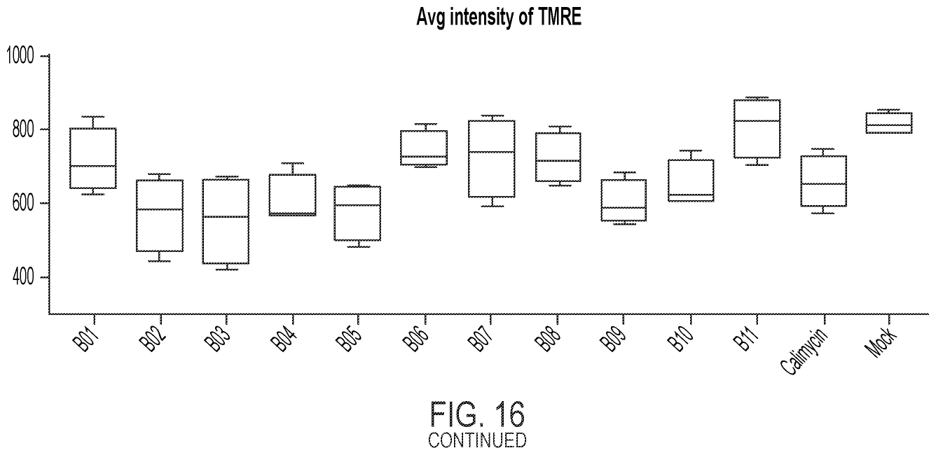

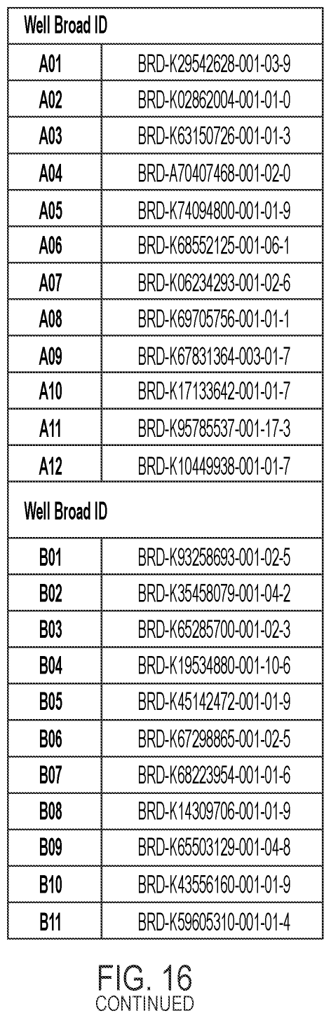

The impact of the compounds on mitochondrial motility was tested, given the observed mitochondrial phenotype. In an automated imaging assay of rat cortical neurons, several compounds were found with decreased mitochondrial motility; and none increased motility (FIG. 16). Although the latter is preferred due to therapeutic potential, this result provided evidence that the virtual screening strategy, applied to a larger set of compounds, might identify novel motility-promoting compounds. We found 3 of the 23 compounds suppress motility but do not decrease mitochondrial membrane potential; this is a much higher hit rate (13.0%) than in our prior screen of 3,280 bioactive compounds, which yielded two such compounds (0.06%).

### Example 6: Discovery of Small Molecules Modulating the Hippo Pathway

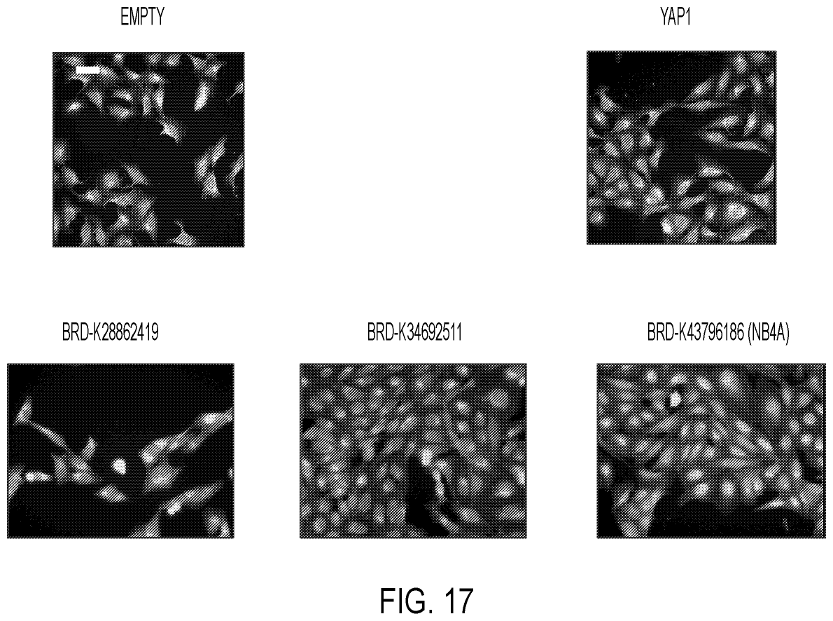

The Hippo pathway plays a key role in development, organ size regulation, and tissue regeneration. Small molecules that alter its activity are highly sought-after for basic research and as potential therapeutics for cancer and other diseases. We tested 30 compounds whose Cell Painting profile matched (25 compounds) or opposed (5 compounds) the overexpression of the Hippo pathway effector Yes-associated protein 1 (YAP1), which were explored previously as identified in Table 1 and Table 2 (FIG. 17). One hit, fipronil, has a known tie to the Hippo pathway: its impact on mRNA profiles matches that of another calcium channel blocker, ivermectin, a potential YAP1 inhibitor (99.9 connectivity score in the Connectivity Map). After identifying 5 promising compounds in a cell proliferation assay in KP230 cells (described later), we focused on the three strongest in various assays and cell contexts, as follows.

N-Benzylquinazolin-4-amine (NB4A, BRD-K43796186) is annotated as an EGFR inhibitor and shares structural similarity with kinase inhibitors. NB4A showed activity in 30 of 606 assays recorded in PubChem, one of which detected inhibitors of TEAD-YAP interaction in HEK-TIYL cells. Its morphological profile positively correlated with that of YAP1 overexpression (0.46) and, consistently, negatively correlated with overexpression of STK3/MST2 (−0.49), a known negative regulator of YAP1.

Because the Hippo pathway can regulate the pluripotency and differentiation of human pluripotent stem cells (hPSCs), the effect of NB4A in H9 hPSCs was investigated. NB4A did not affect YAP1 expression but increased the expression of YAP1 target genes (CTGF and CYR61) in a dose-dependent manner (FIG. 18A), confirming that NB4A impacts the Hippo pathway. Accordingly, NB4A increased YAP1 nuclear localization (FIG. 18B) and decreased YAP1 S127 phosphorylation, which promotes YAP1 cytoplasmic sequestration (FIG. 18C).

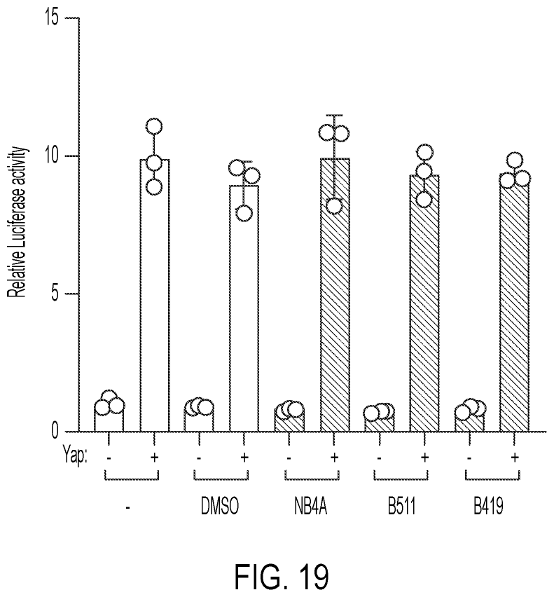

Effects of NB4A on YAP1 mRNA expression were not universal across cell types, consistent with the Hippo pathway's known context-specific functions. In most cell types represented in the Connectivity Map, YAP1 mRNA is unaffected, but in HT29 cells, YAP1 mRNA is up-regulated after six hours of NB4A treatment (z-score=3.16; also z-score=2.04 for TAZ) and in A375 cells, YAP1 mRNA is slightly down-regulated (at 6 and 24 hours; z-score ˜-0.7). NB4A had no effect in a 48h YAP1-responsive reporter assay in HEK-293 cells (FIG. 19).

Compounds influencing the Hippo pathway might be therapeutic for undifferentiated pleomorphic sarcoma (UPS), an aggressive mesenchymal tumor that lacks targeted treatments. In UPS, YAP1 promotes tumorigenesis and is inversely correlated with patient survival. To assess the impact of NB4A on the Hippo pathway, we treated KP230 cells, derived from a mouse model of UPS. In these cells, NB4A did not regulate transcription of Yap1, its sarcoma target genes (Foxm1, Ccl2, Hbegf, Birc5, and Rela), nor Yap1's negative regulator, angiomotin (Amot) (data not shown). Instead, pathways such as interferon alpha and gamma responses were up-regulated, whereas pathways such as the epithelial-mesenchymal transition, angiogenesis, and glycolysis were down-regulated, according to RNA sequencing and gene set enrichment analysis (FIG. 18D, FIG. 20A-D). Nevertheless, we identified impact on the Hippo pathway: Yap1 protein levels were reduced after 72 hours of treatment (FIGS. 18E-F and FIG. 18H). NB4A also significantly attenuated Yap1 nuclear localization (FIGS. 18G-H), which, without wishing to be bound by theory is known to reduce Yap1 ability to impact transcription.

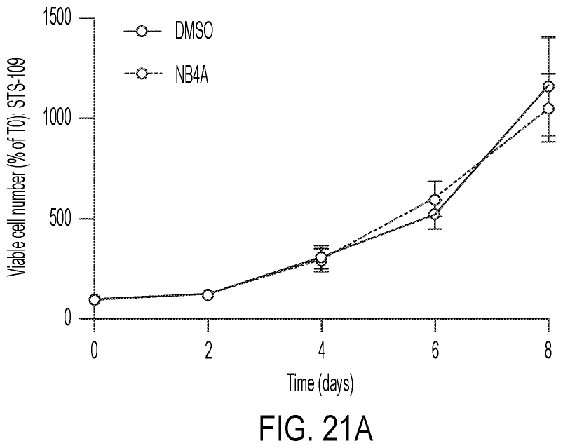

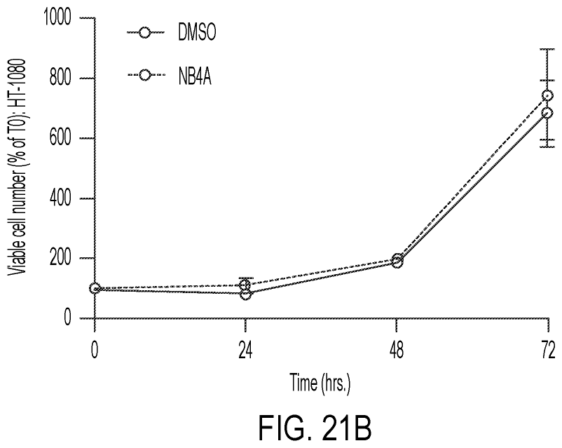

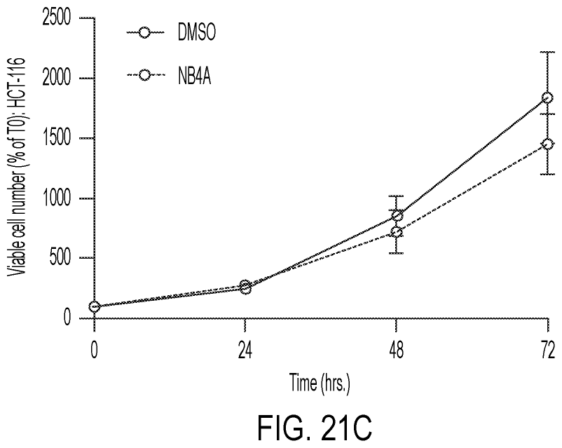

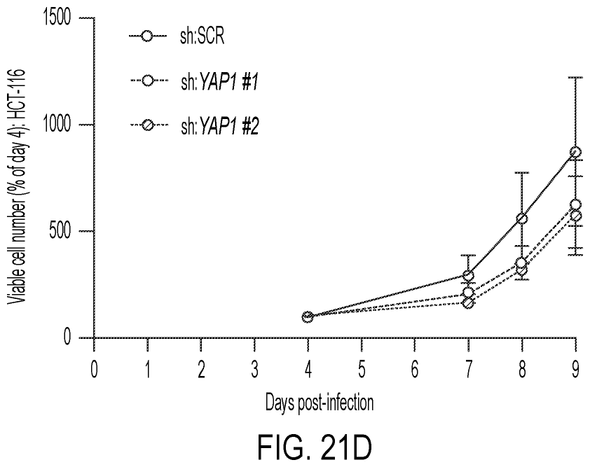

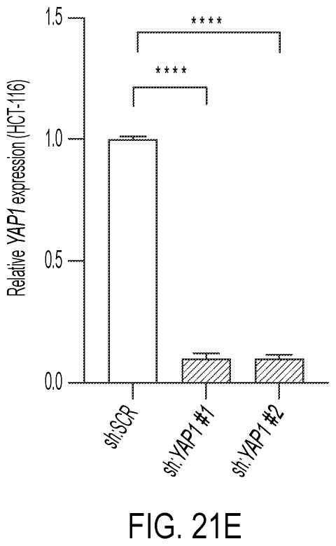

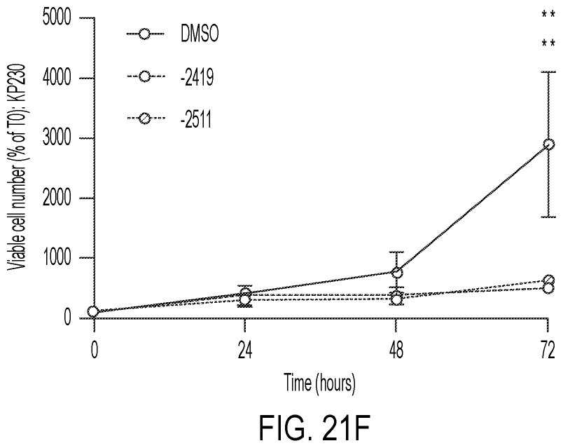

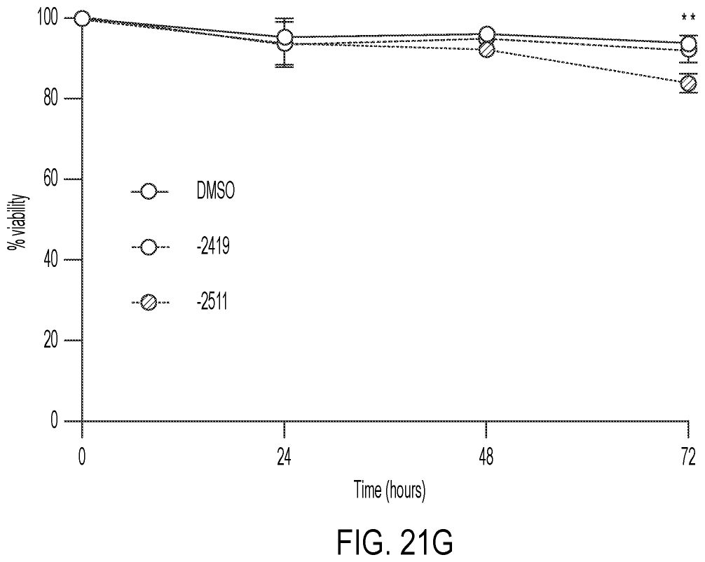

Genetic and pharmacologic inhibition of Yap1 suppresses UPS cell proliferation in vitro and tumor initiation and progression in vivo. Consistent with being a Hippo pathway regulator, NB4A inhibited the proliferation of two YAP1-dependent cell lines: KP230 cells and TC32 human Ewing's family sarcoma cells (FIG. 18I). NB4A did not affect the proliferation of two other YAP1-dependent lines, STS-109 human UPS cells (FIG. 21A) and HT-1080 fibrosarcoma cells (FIG. 21B), nor YAP1-independent HCT-116 colon cancer cells (FIG. 21C-E). Interestingly, NB4A treatment did not exhibit overt toxicity by trypan blue staining in any of these (not shown), suggesting it may inhibit cell proliferation by a mechanism other than eliciting cell death.

Finally, we investigated two structurally similar compounds (BRD-K28862419 and BRD-K34692511, distinct from NB4A's structure) whose Cell Painting profiles negatively correlated with YAP1's overexpression profile (−0.43 for BRD-K28862419 and −0.45 for BRD-K34692511) and positively correlated with TRAF2 overexpression (0.41 for BRD-K28862419 and 0.29 for BRD-K34692511) (FIG. 17). Compounds are shown in Table 5.

TABLE 5

| Compound | | |
|---|---|---|
| ID | Structure | Name |
| NB4A | 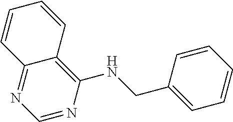 [SMILES: c1ccc(CNc2ncnc3ccccc23)cc1] | N-benzylquinazolin-4-amine |
| BRD- K34692511 (Cmpd. 3) | 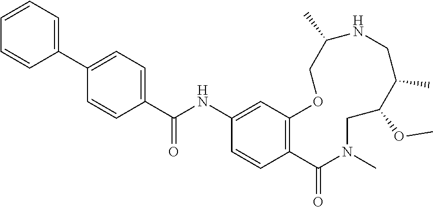 [SMILES: CO[C@@H]1CN(C)C(=O)c2ccc(NC(=O)c3ccc(-c4ccccc4)cc3)cc2OC[C@H](C)NC[C@@H]1C] | N-[(4S,7S,8S)-8-methoxy-4,7,10- trimethyl-11-oxo-2-oxa-5,10- diazabicyclo[10.4.0]hexadeca- 1(12),13,15-trien-15-yl]-4- phenylbenzamide |
| BRD- K28862419 (Cmpd. 4) | 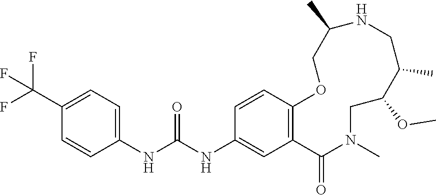 [SMILES: CO[C@@H]1CN(C)C(=O)c2cc(NC(=O)Nc3ccc(C(F)(F)F)cc3)ccc2OC[C@@H](C)NC[C@@H]1C] | 1-[(4R,7S,8S)-8-methoxy-4,7,10- trimethyl-11-oxo-2-oxa-5,10- diazabicyclo[10.4.0]hexadeca- 1(12),13,15-trien-14-yl]-3-[4- (trifluoromethyl)phenyl]urea |

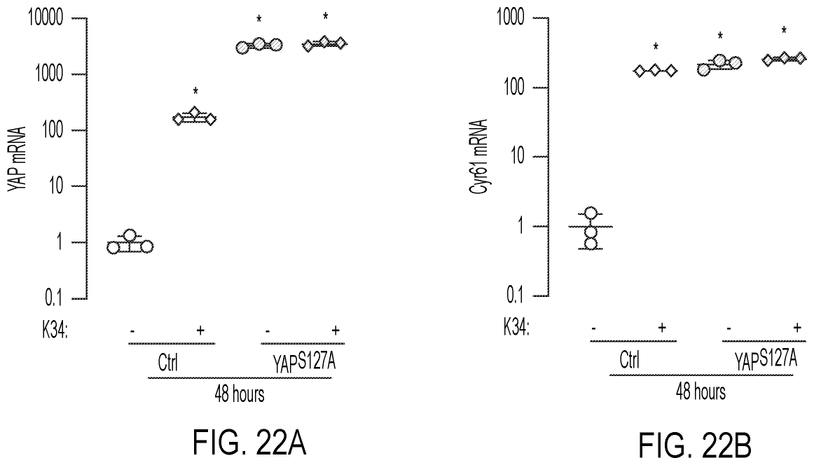

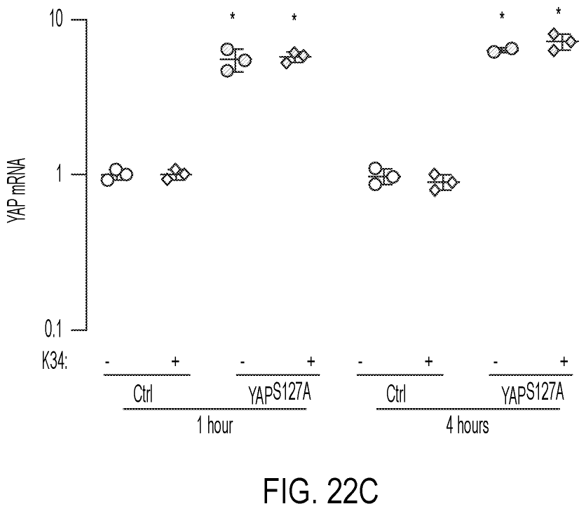

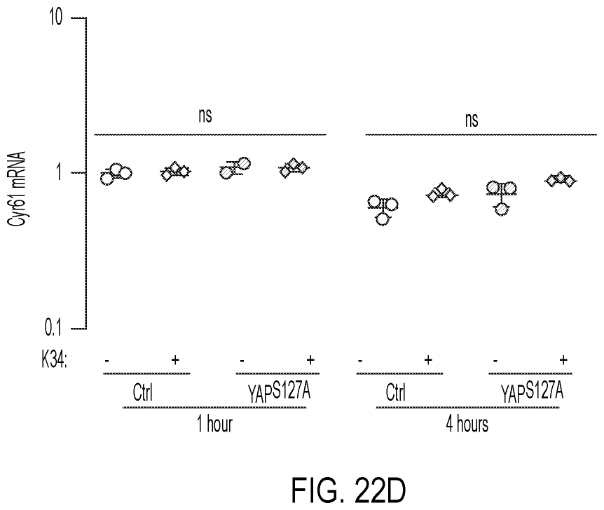

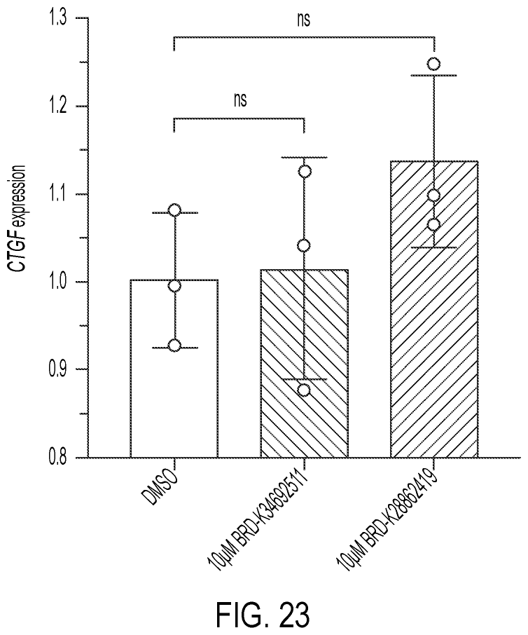

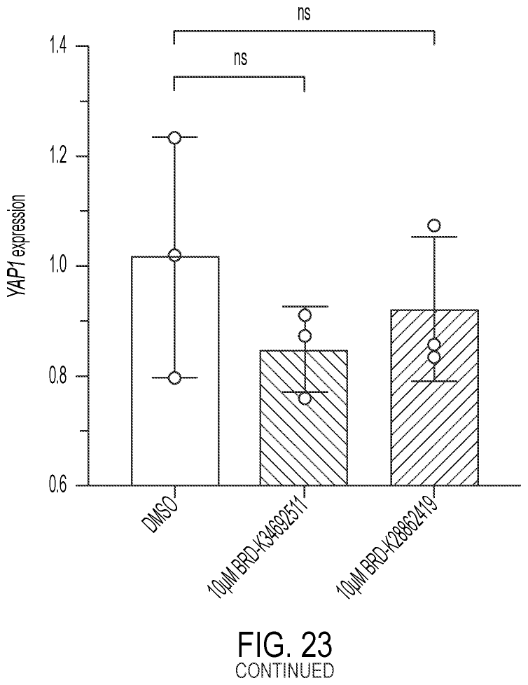

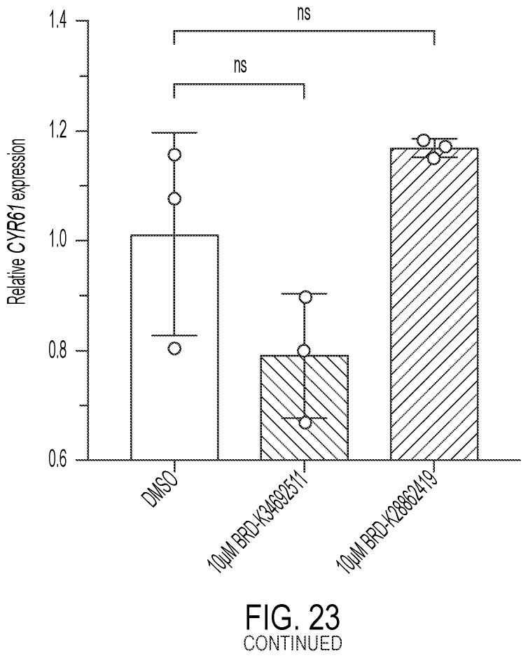

The impact of each compound on the Hippo pathway using mesenchymal lineage periosteal cells isolated from 4-day old femoral fracture callus from mice with DOX-inducible YAP-S127A. BRD-K34692511 substantially upregulated mRNA levels of relevant Hippo components including Yap1 and Cyr61 after 48 hours of treatment, but not at 1 and 4 hours (FIGS. 22A-D). By contrast, the compounds had no effect on YAP1 or its target genes in H9 hPSCs (FIG. 23), nor in a 48 h TEAD reporter assay in HEK-293 cells (FIG. 19).

Like NB4A, the effects of these compounds on proliferation were cell-type specific. In the U2OS Cell Painting images, BRD-K28862419 reduced proliferation (−2.0 st dev). Per PubChem, it inhibits cell proliferation in HEK293, HepG2, A549 cells (AC50 5-18 μM) and it inhibits PAX8, which is known to influence TEAD/YAP signaling. BRD-K34692511 had none of these impacts. Interestingly, both compounds inhibited KP230 cell proliferation (FIG. 21F). Also noteworthy, BRD-K28862419 modestly yet significantly reduced KP230 cell viability (FIG. 21G), indicating its mechanism of action and/or therapeutic index may differ from that of NB4A and BRD-K34692511.

In summary, although deconvoluting the targets and behaviors of these compounds in various cell contexts remains to be further ascertained, we conclude that the strategy identified compounds that modulate the Hippo pathway. This demonstrates that, although the directionality and cell specificity will typically require further study, image-based pathway profiling can identify modulators of a given pathway.

Materials and Methods

p38a experiments

Cell Painting profiles for two wild-type variants of p38a (MAPK14) were averaged to create a p38a Cell Painting profile. 20 compounds whose Cell Painting profile correlated positively or negatively to that of p38a overexpression were selected in addition to 14 “non-correlated compounds” defined as having an absolute value of correlation of less than 0.2 as negative/neutral controls. The compounds were tested for their influence on p38a activity using the RPE1-p38 kinase translocation reporter (KTR) line that was previously generated. This cell line has been tested and confirmed to be negative for mycoplasma contamination, but not necessarily authenticated. P38 activity is measured by phosphorylation of its substrate, MEF2C, which is preferentially phosphorylated by p38 α, while p380 and p386 contribute less. RPE1-p38KTR cells were cultured in DMEM/F12 medium supplemented with 10% Fetal Bovine Serum at 37C in a humidified atmosphere with 5% CO₂. 1000 cells were plated in 96-well plates and treated with 1p M or 10 μM of each compound (n=4 per concentration per compound, no replicates) for 48 hours. Only the middle 60 wells were used to prevent potential confounds from the edge effect. Cells were then fixed in 4% paraformaldehyde for 10 min, followed by permeabilization in cold methanol at −20C for 5 min. Cells were stained with 0.4 μg/mL Alexa Fluor 647 carboxylic acid, succinimidyl ester for 2 hr at RT, followed by 1 μg/mL DAPI for 10 min at RT to facilitate the segmentation of individual cells. p38a activity in single cells was calculated using the ratio of the median intensity of the p38-KTR in a 5-pixel-wide cytoplasmic ring around the nucleus to the median intensity of the p38-KTR in the nucleus. p38a activity measurements were normalized to DMSO conditions within the same plate. The Student's t-test Kolomogorov-Smirnov (KS) test was used to assess the score and significance of the single cell distributions of p38a activity for each compound relative to control. Even for the positivve control known inhibitor, the effect sizes are small. When reporting hits from this assay, KS test and t-test p-values were adjusted to control the false discovery rate using the Benjamini-Hochberg method, using the p.adjust method=‘BH” in R.

PPARGC1A (PGC-1α) Experiments

Reporter assays: To measure PGC-1α activity related to PPARG, RT112/84 cells expressing nuclear GFP (pTagGFP2-H2B, Evrogen) and engineered with the NanoLuc gene cloned into the 3′ UTR of the FABP4 (as described in Usui, M., et al., Genes Cells 21 (2016): 302-310 (2016), which is hereby incorporated by reference in its entirety) for study. These NanoLuc gene cloned RT112/84 cells were plated in 384-well plates at ˜10,000 cells/well. Cells were dosed with indicated compounds in the absence or presence of EC50 of PPARG agonist, rosiglitazone, using an HP D300 digital dispenser. The following day nuclei were counted for normalization (IncuCyte S3, Essen Bioscience) and the reporter activity was evaluated using the NanoGlo Luciferase Assay System (Promega). Normalized data is reported as NanoGlo arbitrary light units divided by cell number. PPARG agonist, rosiglitazone, and inhibitor, T0070907, were obtained from Tocris and included as controls.

To measure effects on PGC1a/ERRalpha, 293T cells were co-transfected with Gal4-ERRα, with and without PGC-1a (pCDNA3.1-Flag-HA-PGC-1α) kind gifts from Pere Puigserver, in combination with the Gal4 UAS reporter construct, pGL4.35 [luc2P/9XGAL4UAS/Hygro] (Promega) modified by subcloning the HSV-TK promoter into the unique HindIII site that is downstream of the 9xGal4 UAS sites, in addition to a Renilla luciferase expression vector pRL (Promega) for normalization. Cells were dosed with compounds and 24 hours later, plates were analyzed using Dual-Glo Luciferase Assay System (Promega). Normalized light units are reported as Firefly luciferase divided by Renilla luciferase. ERRalpha modulators XCT790, Daidzein, and Biochanin A were obtained from Cayman Chemical and included as controls.

High content mitochondrial motility screen: Mitochondrial motility was measured as described in Shlevkov, E. et al., Cell Rep. 28 (2019): 3224-3237.e5. Briefly, we plated E18 rat cortical neurons in the middle 60 wells of 96 well plates (Greiner)—40,000 cells per well in 150 μl enriched Neurobasal media. Neurons were transfected with mito-DsRed at DIV7 using Lipofectamine2000 (Life Technologies). Plating and transfection were all done using an Integra VIAFLO 96/384 automated liquid handler. At DIV9, test compounds were added into wells to achieve a final concentration of 10 μM each (4 wells per compound), as well at 10 μM calcimycin for neg. control, and DMSO only for mock treatment. Following a 1-2 hour incubation, plates were imaged on a ArrayScan XTI (Thermo Fisher). Mitochondrial motility data was extrapolated from imaging data using a MATLAB and CellProfiler based computational pipeline. Compounds A01-A12 were tested on one plate; B01-B11 were tested separately on another plate on the same day. The experiment was repeated twice in different weeks. In the second week, TMRE was added to all wells after imaging was completed (20 min, then 2 washes) and imaged to measure mitochondrial membrane potential in order to determine mitochondrial and cell health.

YAP1-Related Compounds

For the initial experiments, quality control of the compounds revealed that purity was 88% for A15 (BRD-K34692511-001-01-9), 81% for A05 (BRD-K28862419-001-01-9), and >99% for E07 (BRD-K43796186-001-01-1). For subsequent experiments in the Eisinger lab, BRD-K43796186 (NB4A) was ordered from MuseChem (cat. #M189943) and for the Kiessling lab, from Ambinter (Cat #Amb2554311).

YAP1 cell culture and treatments: Eisinger lab: Murine KP230 cells, a Yap1-dependent cancer cell line, were derived from a tumor from the KP mouse model (Kras^{G12/D}; Trp53^{f/fl}), as described in Eisinger-Mathason, T. S. K. et al., Proc. Natl. Acad. Sci. U.S.A 112 (2015): E3402-11, which is hereby incorporated by reference in its entirety. STS-109 UPS cells were derived from a human UPS tumor and validated by Rebecca Gladdy, MD (Sinai Health System, Toronto, Ontario, Canada). TC32 cells were a gift from Patrick Grohar, MD, PhD (Children's Hospital of Philadelphia). HT-1080, HCT-116, and HEK293T cells were purchased from ATCC. KP230, HT-1080, and HEK-293T cells were grown in DMEM with 10% FBS, 1% L-glutamine, and 1% penicillin/streptomycin (P/S). STS-109 cells were cultured in DMEM with 20% FBS, 1% L-glutamine, and 1% P/S. TC32 cells were grown in RPMI with 10% FBS, 1% L-glutamine, and 1% P/S. HCT-116 cells were cultured in McCoy's 5A medium with 10% FBS and 1% P/S. All cells were confirmed to be negative for mycoplasma contamination and were maintained in an incubator at 37C with 5% CO₂. For experimental purposes, cells were cultured for up to 20 passages before being discarded, and were grown to approximately 50% confluence to circumvent the effects of high cell density on Yap1 expression and activity. All cell lines in the Eisinger laboratory were treated with 10 μM of each inhibitor or an equivalent volume of DMSO every 24 hours for 3 days, except for STS-109 cells, which were treated daily for 8 days.

H9 hPSCs (WiCell) were maintained on vitronectin (Thermo Fisher)-coated plates in Essential 8 (E8) medium. The cells were routinely passaged using 0.5 mM EDTA and treated with 5 μM Y-27632 dihydrochloride (Tocris) on the first day. For testing the effects of the small molecules, H9 hPSCs were seeded at 50K cells/cm² on vitronectin-coated plates in E8 medium supplemented with 5 μM Y-27632 dihydrochloride (day 0). On day 1, the medium was switched to E8 medium. On day 2, the medium was switched to E8 medium supplemented with the small molecules. Following overnight incubation, the cells were collected for subsequent analysis on day 3.

Murine periosteal cells were isolated from a transgenic mouse model (CMV-Cre; R26R-rtTA^{fl}; tetO-YAP^{S127A}) in which YAP1 can be activated in a doxycycline inducible manner (Camargo 2011). This mouse model expresses a mutant form of YAP1 (YAP^{S127A}) that escapes degradation. Cells were cultured in a-MEM with 15% Fetal Bovine serum (S11550, R&D Systems), 1% GlutaMAX-I (Gibco, 35050-061) and 1% Penicillin/Streptomycin (Gibco, 15140-122).

YAP1-related lentiviral production:_Knockdown of YAP1 in HCT-116 cells was performed with shRNAs (TRC clone IDs: TRCN0000107266 and TRCN0000107267); a scrambled shRNA was used as a negative control. shRNA plasmids (Dharmacon) were packaged using the third-generation lentiviral vector system (pVSV-G, pMDLG, and pRSV-REV; Addgene) and expressed in HEK-293T cells using Fugene 6 transfection reagent (Promega). Virus-containing supernatants were collected 24 and 48 hours after transfection and concentrated 40-fold by centrifugation with polyethylene glycol 8000.

YAP1-Related Proliferation Assays

NB4A treatment: Cells were treated with 10 μM of each inhibitor or an equivalent volume of DMSO every 24 hours for 3-8 days, and counted with a hemocytometer with trypan blue exclusion daily (KP230, HT-1080, TC32, HCT-116), or every 2 days (STS-109).

shRNA-mediated YAP1 knockdown: HCT-116 cells were infected with YAP1 shRNA-encoding lentiviruses in the presence of 8 μg/mL polybrene (Sigma). Antibiotic selection (3 μg/mL puromycin) was performed after 48 hours, after which cells were cultured for an additional 48 hours. Cells were then trypsinized, seeded under puromycin-selection conditions, and counted with a hemocytometer with trypan blue exclusion on days 7, 8, and 9 post-infection.

YAP1-related qRT-PCR: Total RNA from cultured cells was isolated with the QIAGEN RNeasy mini kit, and cDNA was synthesized with the High-Capacity RNA-to-cDNA kit (Life Technologies). qRT-PCR analysis was performed with TaqMan “best coverage” probes on a ViiA7 instrument. Hypoxanthine phosphoribosyltransferase (HPRT) and succinate dehydrogenase subunit A (SDHA) were used as endogenous controls. Relative expression was calculated using the ddCt method.

The RNA was extracted using TRIzol (Life Technologies) and Direct-zol™ RNA MiniPrep kit (Zymo Research) as per manufacturer instructions. The RNA was reverse transcribed using iScript cDNA synthesis kit (Bio-Rad). The qPCR was performed on CFX Connect (Bio-Rad) using iTaq Universal SYBR Green Supermix (Bio-Rad). GAPDH was used as a reference gene for normalization. The relative gene expression levels were determined using the ddCt method. The primer sequences used are listed in Table 6.

TABLE 6

| Gene | | |
|---|---|---|
| name | Forward primer | Reverse primer |
| GAPDH | GTGGTCTCCTCTGACTTCA | CCTGTTGCTGTAGCCAAATT |
| | AC (SEQ ID NO: 3) | C (SEQ ID NO: 4) |
| YAP1 | GCTGCCACCAAGCTAGATA | GTGCATGTGTCTCCTTAGAT |
| | A (SEQ ID NO: 5) | CC (SEQ ID NO: 6) |
| CTGF | GTGCATCCGTACTCCCAAA | CTCCACAGAATTTAGCTCGG |
| | (SEQ ID NO: 7) | TAT (SEQ ID NO: 8) |
| CYR61 | AGCCTCGCATCCTATACAA | TTCTTTCACAAGGCGGCACT |
| | CC (SEQ ID NO: 9) | C (SEQ ID NO: 10) |
| Yap1 | GATGTCTCAGGAATTGAGA | CTGTATCCATTTCATCCACA |
| | AC (SEQ ID NO: 11) | C (SEQ ID NO: 12) |
| Cyr61 | CTGCGCTAAACAACTCAAC | GCAGATCCCTTTCAGAGCGG |
| | GA (SEQ ID NO: 13) | (SEQ ID NO: 14) |

To induce YAP^{S127A}, 1p M doxycycline was added to the cell culture medium for 48 hours. This was used as a positive control to compare YAP1 mRNA expression. Cells were also treated with BRD-K34692511-001-01-9 at 5 μM. mRNA was isolated from cells (n=3/group/time point) at 1, 4 or 48 hours after treatment using Qiagen RNeasy Mini kit (Qiagen, 74106). cDNA was prepared as per the manufacturer's protocol using the High-Capacity Reverse Transcription kit (Thermofisher scientific, 4368814). qPCR analysis was performed using the QuantStudio 6 Pro Real-Time PCR System.

YAP1-related reporter assay: HEK293T cells were co-transfected using Lipofectamine 3000 (Thermo Fisher) with a TEAD luciferase reporter construct, 8×GTIIC-luciferase (Addgene plasmid #34615), a plasmid expressing Renilla Luciferase from a CMV promoter as a transfection control, along with a plasmid expressing 3×Flag-tagged wild-type YAP1 from a CMV promoter (pCMV5 backbone). Following transfection the cells were immediately treated with 0.2% DMSO, 10 μM NB4A, BRD-K34692511 or BRD-K28862419 and then lysed 48 hours later. Lysates were examined using the Dual-Luciferase Reporter Assay System (Promega) according to the manufacturer's protocol and measured using a SpectraMax iD3 plate reader (Molecular Devices). Firefly Luciferase activity from the TEAD reporter was normalized to Renilla Luciferase activity and then plotted as relative values.

YAP1-related RNA-sequencing and data analysis: Total RNA from cultured cells was isolated with the QIAGEN RNeasy Mini Kit with on-column DNase digestion. RNA quality checks were performed with an Agilent 2100 Bioanalyzer (Eukaryotic Total RNA Nano kit). Library preparation (500 ng input RNA) was performed with the NEBNext Poly(A) mRNA Magnetic Isolation Module (#E7490) with SPRIselect Beads (Beckman Coulter), the NEBNext Ultra II Single-End RNA Library Prep kit (#7775S), and the NEBNext Multiplex Oligos for Illumina (Index Primers Set 1) according to the manufacturer's instructions. Library size was confirmed with an Agilent 2100 Bioanalyzer (DNA1000 chip). Pooled libraries were diluted to 1.8 μM (concentrations checked with the Qubit Fluorometer high-sensitivity assay, Thermo Fisher), and sequenced on an Illumina NexSeq 500 instrument with the NexSeq 500 75-cycle high-output kit.

For data analysis, FASTQ files were generated with the bcl2fastq command line program (Illumina). Transcript alignment was performed with Salmon. Differential expression analysis (NB4A- vs. DMSO-treated cells) was performed with the DESeq2 R package. DESeq2 “stat” values for each gene were used as inputs to pre-ranked GSEA, where enrichment was tested against the Hallmark gene sets from the Molecular Signatures Database (MSigDB). Access to sequencing data is discussed in the data availability section.

YAP1-related Western blotting: The cells were lysed in RIPA buffer (Pierce) supplemented with Halt Protease inhibitor cocktail and Halt Phosphatase inhibitor cocktail. Cells were lysed in hot Tris-SDS buffer (pH 7.6) and boiled for 5 minutes at 95° C. The protein concentration of each sample was quantified using the Pierce BCA protein assay (Thermo Fisher). The proteins were resolved by SDS-PAGE and transferred to PVDF membranes using the Trans-Blot Turbo Transfer system (Bio-Rad). The membranes were blocked in 5% non-fat milk in TBS-T for up to 1 hour at room temperature and incubated with primary antibodies in 5% bovine serum albumin in TBS-T overnight at 4° C. Then, the membranes were incubated with HRP-conjugated anti-rabbit IgG secondary antibodies at 1:10000 (Kiessling lab) or 1:2500 (Cell Signaling Technology [CST] #7074) for 1 hour at RT and developed in the ChemiDoc MP Imaging system or on autoradiography film using ECL Prime reagent (Amersham). The primary antibodies and dilutions used are: anti-YAP1 (CST 4912S and CST 14074) at 1:1000, anti-phospho-YAP1-S127 (CST 4911S) at 1:1000, and anti-GAPDH (CST 5174 and CST 2118) at 1:15000 and 1:1000, respectively.

YAP1-related immunofluorescence and image analysis: Cells were grown on poly-L-lysine-coated chamber slides were fixed in 4% PFA (15 minutes at room temperature), permeabilized with 0.5% Triton-X100/PBS (15 minutes at room temperature), and blocked with 5% goat serum (Vector Laboratories S-1000; 1 hour at room temperature). Cells were then incubated with anti-Yap1 primary antibodies (CST #14074; 1:1000) diluted in blocking buffer overnight at 4° C. Subsequently, cells were incubated with Alexa Fluor 488-conjugated secondary antibodies (4 ug/mL in blocking buffer; Thermo Fisher Scientific #A-11008) for 1 hour at room temperature. Coverslip mounting was performed with ProLong Gold Antifade reagent with DAPI. Images (5 fields per condition for each of 3 independent experiments) were acquired with a Nikon Eclipse Ni microscope and Nikon NES Elements software. Image analysis was performed with Fiji as follows: For nuclear staining intensity, watershed analysis of DAPI channel images (8-bit) was performed to “separate” nuclei that appeared to be touching. Nuclei were then converted to regions of interest (ROIs) that were “applied” to the corresponding GFP channel image (8-bit format). Analysis of staining intensity in these nuclear ROIs was then performed, excluding objects smaller than 100 pixels² (integrated density normalized to number of nuclei). A similar process was followed to determine whole-cell staining intensity: using 8-bit GFP channel images, cells were distinguished from background via thresholding, and converted to ROIs that were applied back to the 8-bit GFP channel images. Analysis of staining intensity (integrated density normalized to number of nuclei) was then performed in these ROIs, excluding objects smaller than 500 pixels². The ratio of nuclear to total Yap1 expression was determined after subtracting out background GFP signal from no-primary antibody controls.

The cells were fixed with 4% formaldehyde for 15 mins at room temperature. The cells were permeabilized and blocked with PBS containing 2% BSA and 0.1% Triton-X100. The cells were incubated with a primary antibody against YAP1 (sc-101199) at 1:1000 dilution in a blocking buffer overnight at 4° C. Then, a goat anti-mouse Alexa Fluor 488 conjugated secondary antibody was added for 1 hour at room temperature. The nuclei were counterstained with 4′,6-diamidino-2-phyenylindole (DAPI) dilactate (Molecular Probes). Images were collected with Olympus FV1200 microscope and analyzed with CellProfiler. Briefly, nuclei and cell bodies were segmented using DAPI and YAP channels respectively. The cell cytoplasm was determined as the region outside nuclei but within the cell bodies. Then, an average pixel intensity of YAP in the nucleus and cytoplasm was calculated to determine YAP translocation

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for inhibiting proliferation or survival of a neoplasia associated with an NF-κB pathway in a cell, the method comprising contacting the cell with a compound selected from the group consisting of:
N-[[(8R,9S)-6-[(2R)-1-hydroxypropan-2-yl]-8-methyl-5-oxo-10-oxa-1,6,13,14 tetrazabicyclo[10.2.1]pentadeca-12(15),13-dien-9-yl]methyl]-N-methyl-4 phenoxybenzenesulfonamide,
(4S,5R)-5-((dimethylamino)methyl)-2-((R)-1-hydroxypropan-2-yl)-4-methyl-8-(pyridin-2-ylethynyl)-2,3,4,5-tetrahydrobenzo[b][1,4,5]oxathiazocine 1,1-dioxide,
N-[(4S,7S,8S)-8-methoxy-4,7,10-trimethyl-11-oxo-2-oxa-5,10-diazabicyclo[10.4.0]hexadeca-1(12),13,15-trien-15-yl]-4-phenylbenzamide,
1-[(4R,7S,8S)-8-methoxy-4,7,10-trimethyl-11-oxo-2-oxa-5,10-diazabicyclo[10.4.0]hexadeca-1(12),13,15-trien-14-yl]-3-[4-(trifluoromethyl)phenyl]urea,
2-[(3 S,6aR,8S,10aR)-3-hydroxy-1-(3-methoxyphenyl)sulfonyl-3,4,6,6a,8,9,10,10a-octahydro-2H-pyrano[2,3-c][1,5]oxazocin-8-yl]-1-(4-phenyl-1-piperazinyl)ethanone, 1-pyridin-4-yl-3-(2,4,6-trichlorophenyl)urea,
4-(5,7,7,10,10-pentamethyl-8,9-dihydronaphtho[2,3-b][1,4]benzodiazepin-13-yl)benzoic acid (LE-135),
1-(3,3a,4,5,6,6a-hexahydro-1H-cyclopenta[c]pyrrol-2-yl)-3-(4-methylphenyl)sulfonylurea,
5-(1,4-diazepan-1-ylsulfonyl)-2H-isoquinolin-1-one,
1-[(3,4-dimethoxyphenyl)methyl]-6,7-dimethoxyisoquinoline,
5-(4-chlorophenyl)-6-ethylpyrimidine-2,4-diamine,
7-hydroxy-3-(4-hydroxyphenyl)chromen-4-one,
N-benzylquinazolin-4-amine,
(2R,3R,3aS,9bS)-7-(1-cyclohexenyl)-N-(cyclopropylmethyl)-3-(hydroxymethyl)-6-oxo-1,2,3,3a,4,9b-hexahydropyrrolo[2,3-a]indolizine-2-carboxamide,
N-[(1R,3R,4aS,9aR)-3-[2-[(3-fluorophenyl)methylamino]-2-oxoethyl]-1-(hydroxymethyl)-3,4,4a,9a-tetrahydro-1H-pyrano[3,4-b]benzofuran-6-yl]-1,3-benzodioxole-5-carboxamide,
(1S,9R,10R,11R)-11-N-ethyl-10-(hydroxymethyl)-5-(2-methoxyphenyl)-6-oxo-12-N-propyl-7,12-diazatricyclo[7.2.1.02,7]dodeca-2,4-diene-11,12-dicarboxamide,
N-[(1S,3S,4aR,9aS)-1-(hydroxymethyl)-3-[2-oxo-2-(1-piperidinyl)ethyl]-3,4,4a,9a-tetrahydro-1H-pyrano[3,4-b]benzofuran-6-yl]-4-oxanecarboxamide,
N-[(5S,6S,9S)-8-(cyclopropylmethyl)-5-methoxy-3,6,9-trimethyl-2-oxo-11-oxa-3,8-diazabicyclo[10.4.0]hexadeca-1(12),13,15-trien-14-yl]-2-fluorobenzamide,
N-[(4R,7S,8R)-8-methoxy-4,7,10-trimethyl-11-oxo-5-(1,3-thiazol-2-ylmethyl)-2-oxa-5,10-diazabicyclo[10.4.0]hexadeca-1(12),13,15-trien-14-yl]cyclohexanecarboxamide,
2-[(3 S,6aR,8R,10aR)-1-(1,3-benzodioxol-5-ylmethyl)-3-hydroxy-3,4,6,6a,8,9,10,10a-octahydro-2H-pyrano[2,3-c][1,5]oxazocin-8-yl]-1-piperidin-1-ylethanone,
2-[(1R,3R,4aS,9aR)-1-(hydroxymethyl)-6-[(3-methoxyphenyl)sulfonylamino]-3,4,4a,9a-tetrahydro-1H-pyrano[3,4-b]benzofuran-3-yl]acetic acid methyl ester,
4-fluoro-N-[(2R,3R)-5-[(2R)-1-hydroxypropan-2-yl]-3-methyl-2-(methylaminomethyl)-6-oxo-3,4-dihydro-2H-1,5-benzoxazocin-10-yl]benzenesulfonamide,
N-[(2S,3S,6R)-2-(hydroxymethyl)-6-[2-oxo-2-(1,3-thiazol-2-ylamino)ethyl]oxan-3-yl]-3-piperidin-1-ylpropanamide,
N-[(4S,7R,8R)-8-methoxy-4,7,10-trimethyl-11-oxo-5-(phenylmethyl)-2-oxa-5,10-diazabicyclo[10.4.0]hexadeca-1(12),13,15-trien-14-yl]butanamide,
2-[(2R,3R,6S)-3-[[(2,5-difluoroanilino)-oxomethyl]amino]-2-(hydroxymethyl)-3,6-dihydro-2H-pyran-6-yl]-N-[3-(4-morpholinyl)propyl]acetamide,
N-benzyl-2-chloroquinazolin-4-amine,
N-[(2R,3 S,6S)-6-[2-[(4-fluorophenyl)sulfonylamino]ethyl]-2-(hydroxymethyl)oxan-3-yl]oxane-4-carboxamide,
5-amino-1-[2,6-dichloro-4-(trifluoromethyl)phenyl]-4-(trifluoromethane)sulfinyl-1H-pyrazole-3-carbonitrile,
N-[(1S,3S,4aS,9aR)-1-(hydroxymethyl)-3-[2-oxo-2-(pyridin-2-ylmethylamino)ethyl]-3,4,4a,9a-tetrahydro-1H-pyrano[3,4-b][1]benzofuran-6-yl]cyclobutanecarboxamide, and
and 1-[[(8S,9R)-6-[(2S)-1-hydroxypropan-2-yl]-8-methyl-5-oxo-10-oxa-1,6,14,15-tetrazabicyclo[10.3.0]pentadeca-12,14-dien-9-yl]methyl]-1-methyl-3-(3-pyridin-2-yloxyphenyl)urea; or
a pharmaceutically acceptable salt thereof, or a tautomer, stereoisomer, prodrug and/or solvate of any of the foregoing;
thereby inhibiting proliferation or survival of the cell.

2. The method of claim 1, wherein the compound is selected from the group consisting of N-benzylquinazolin-4-amine,
N-[(4S,7S,8S)-8-methoxy-4,7,10-trimethyl-11-oxo-2-oxa-5,10-diazabicyclo[10.4.0]hexadeca-1(12),13,15-trien-15-yl]-4-phenylbenzamide, and
1-[(4R,7S,8S)-8-methoxy-4,7,10-trimethyl-11-oxo-2-oxa-5,10-diazabicyclo[10.4.0]hexadeca-1(12),13,15-trien-14-yl]-3-[4-(trifluoromethyl)phenyl]urea; or
a pharmaceutically acceptable salt thereof, or a tautomer, stereoisomer, prodrug and/or solvate of any of the foregoing.

3. The method of claim 2, wherein the cell is contacted with an effective amount of N-benzylquinazolin-4-amine; or
a pharmaceutically acceptable salt thereof, or a tautomer, stereoisomer, prodrug and/or solvate of any of the foregoing.

4. The method of claim 1, wherein the neoplasia is selected from the group consisting of sarcoma, pancreatic cancer, prostate cancer, head and neck cancer, breast cancer, and liver cancer.

5. The method of claim 1, wherein the cell is a mammalian cell.

6. The method of claim 1, wherein the cell is in vitro or in vivo.

7. A method for inhibiting proliferation or survival of a neoplasia associated with a Hippo pathway in a cell, the method comprising contacting the cell with a compound selected from the group consisting of:
N-[[(8R,9S)-6-[(2R)-1-hydroxypropan-2-yl]-8-methyl-5-oxo-10-oxa-1,6,13,14 tetrazabicyclo[10.2.1]pentadeca-12(15),13-dien-9-yl]methyl]-N-methyl-4 phenoxybenzenesulfonamide,
(4S,5R)-5-((dimethylamino)methyl)-2-((R)-1-hydroxypropan-2-yl)-4-methyl-8-(pyridin-2-ylethynyl)-2,3,4,5-tetrahydrobenzo[b][1,4,5]oxathiazocine 1,1-dioxide,
N-[(4S,7S,8S)-8-methoxy-4,7,10-trimethyl-11-oxo-2-oxa-5,10-diazabicyclo[10.4.0]hexadeca-1(12),13,15-trien-15-yl]-4-phenylbenzamide,
1-[(4R,7S,8S)-8-methoxy-4,7,10-trimethyl-11-oxo-2-oxa-5,10-diazabicyclo[10.4.0]hexadeca-1(12),13,15-trien-14-yl]-3-[4-(trifluoromethyl)phenyl]urea,
2-[(3 S,6aR,8S,10aR)-3-hydroxy-1-(3-methoxyphenyl)sulfonyl-3,4,6,6a,8,9,10,10a-octahydro-2H-pyrano[2,3-c][1,5]oxazocin-8-yl]-1-(4-phenyl-1-piperazinyl)ethanone, 1-pyridin-4-yl-3-(2,4,6-trichlorophenyl)urea,
4-(5,7,7,10,10-pentamethyl-8,9-dihydronaphtho[2,3-b][1,4]benzodiazepin-13-yl)benzoic acid (LE-135),
1-(3,3a,4,5,6,6a-hexahydro-1H-cyclopenta[c]pyrrol-2-yl)-3-(4-methylphenyl)sulfonylurea,
5-(1,4-diazepan-1-ylsulfonyl)-2H-isoquinolin-1-one,
1-[(3,4-dimethoxyphenyl)methyl]-6,7-dimethoxyisoquinoline,
5-(4-chlorophenyl)-6-ethylpyrimidine-2,4-diamine,
7-hydroxy-3-(4-hydroxyphenyl)chromen-4-one,
N-benzylquinazolin-4-amine,
(2R,3R,3aS,9bS)-7-(1-cyclohexenyl)-N-(cyclopropylmethyl)-3-(hydroxymethyl)-6-oxo-1,2,3,3a,4,9b-hexahydropyrrolo[2,3-a]indolizine-2-carboxamide,
N-[(1R,3R,4aS,9aR)-3-[2-[(3-fluorophenyl)methylamino]-2-oxoethyl]-1-(hydroxymethyl)-3,4,4a,9a-tetrahydro-1H-pyrano[3,4-b]benzofuran-6-yl]-1,3-benzodioxole-5-carboxamide,
(1S,9R,10R,11R)-11-N-ethyl-IO-(hydroxymethyl)-5-(2-methoxyphenyl)-6-oxo-12-N-propyl-7,12-diazatricyclo[7.2.1.02,7]dodeca-2,4-diene-11,12-dicarboxamide,
N-[(1S,3S,4aR,9aS)-1-(hydroxymethyl)-3-[2-oxo-2-(1-piperidinyl)ethyl]-3,4,4a,9a-tetrahydro-1H-pyrano[3,4-b]benzofuran-6-yl]-4-oxanecarboxamide,
N-[(5S,6S,9S)-8-(cyclopropylmethyl)-5-methoxy-3,6,9-trimethyl-2-oxo-11-oxa-3,8-diazabicyclo[10.4.0]hexadeca-1(12),13,15-trien-14-yl]-2-fluorobenzamide,
N-[(4R,7S,8R)-8-methoxy-4,7,10-trimethyl-11-oxo-5-(1,3-thiazol-2-ylmethyl)-2-oxa-5,10-diazabicyclo[10.4.0]hexadeca-1(12),13,15-trien-14-yl]cyclohexanecarboxamide,
2-[(3 S,6aR,8R,10aR)-1-(1,3-benzodioxol-5-ylmethyl)-3-hydroxy-3,4,6,6a,8,9,10,10a-octahydro-2H-pyrano[2,3-c][1,5]oxazocin-8-yl]-1-piperidin-1-ylethanone,
2-[(1R,3R,4aS,9aR)-1-(hydroxymethyl)-6-[(3-methoxyphenyl)sulfonylamino]-3,4,4a,9a-tetrahydro-1H-pyrano[3,4-b]benzofuran-3-yl]acetic acid methyl ester,
4-fluoro-N-[(2R,3R)-5-[(2R)-1-hydroxypropan-2-yl]-3-methyl-2-(methylaminomethyl)-6-oxo-3,4-dihydro-2H-1,5-benzoxazocin-10-yl]benzenesulfonamide,
N-[(2S,3S,6R)-2-(hydroxymethyl)-6-[2-oxo-2-(1,3-thiazol-2-ylamino)ethyl]oxan-3-yl]-3-piperidin-1-ylpropanamide,
N-[(4S,7R,8R)-8-methoxy-4,7,10-trimethyl-11-oxo-5-(phenylmethyl)-2-oxa-5,10-diazabicyclo[10.4.0]hexadeca-1(12),13,15-trien-14-yl]butanamide,
2-[(2R,3R,6S)-3-[[(2,5-difluoroanilino)-oxomethyl]amino]-2-(hydroxymethyl)-3,6-dihydro-2H-pyran-6-yl]-N-[3-(4-morpholinyl)propyl]acetamide,
N-benzyl-2-chloroquinazolin-4-amine,
N-[(2R,3 S,6S)-6-[2-[(4-fluorophenyl)sulfonylamino]ethyl]-2-(hydroxymethyl)oxan-3-yl]oxane-4-carboxamide,
5-amino-1-[2,6-dichloro-4-(trifluoromethyl)phenyl]-4-(trifluoromethane)sulfinyl-1H-pyrazole-3-carbonitrile,
N-[(1S,3S,4aS,9aR)-1-(hydroxymethyl)-3-[2-oxo-2-(pyridin-2-ylmethylamino)ethyl]-3,4,4a,9a-tetrahydro-1H-pyrano[3,4-b][1]benzofuran-6-yl]cyclobutanecarboxamide, and
1-[[(8S,9R)-6-[(2S)-1-hydroxypropan-2-yl]-8-methyl-5-oxo-10-oxa-1,6,14,15-tetrazabicyclo[10.3.0]pentadeca-12,14-dien-9-yl]methyl]-1-methyl-3-(3-pyridin-2-yloxyphenyl)urea; or
a pharmaceutically acceptable salt thereof, or a tautomer, stereoisomer, prodrug and/or solvate of any of the foregoing;
thereby inhibiting proliferation or survival of the cell.

8. The A-method of claim 7, wherein the compound is selected from the group consisting of N-benzylquinazolin-4-amine,
N-[(4S,7S,8S)-8-methoxy-4,7,10-trimethyl-11-oxo-2-oxa-5,10-diazabicyclo[10.4.0]hexadeca-1(12),13,15-trien-15-yl]-4-phenylbenzamide, and
1-[(4R,7S,8S)-8-methoxy-4,7,10-trimethyl-11-oxo-2-oxa-5,10-diazabicyclo[10.4.0]hexadeca-1(12),13,15-trien-14-yl]-3-[4-(trifluoromethyl)phenyl]urea; or
a pharmaceutically acceptable salt thereof, or a tautomer, stereoisomer, prodrug and/or solvate of any of the foregoing.

9. The method of claim 8, wherein the cell is contacted with an effective amount of N-benzylquinazolin-4-amine; or
a pharmaceutically acceptable salt thereof, or a tautomer, stereoisomer, prodrug and/or solvate of any of the foregoing.

10. The method of claim 7, wherein the neoplasia is selected from the group consisting of sarcoma, pancreatic cancer, prostate cancer, head and neck cancer, breast cancer, and liver cancer.

11. The method according to claim 10, wherein the neoplasia is undifferentiated pleomorphic sarcoma.

12. The method of claim 7, wherein the cell is a mammalian cell.

13. The method of claim 7, wherein the cell is in vitro or in vivo.

14. A method of treating a neoplasia associated with the NF-κB and/or Hippo pathway in a subject, the method comprising administering to the subject a compound selected from the group consisting of:
(1S,9R,10R,11R)-11-N-ethyl-10-(hydroxymethyl)-5-(2-methoxyphenyl)-6-oxo-12-N-propyl-7,12-diazatricyclo[7.2.1.02,7]dodeca-2,4-diene-11,12-dicarboxamide,
(2R,3R,3aS,9bS)-7-(1-cyclohexenyl)-N-(cyclopropylmethyl)-3-(hydroxymethyl)-6-oxo-1,2,3,3a,4,9b-hexahydropyrrolo[2,3-a]indolizine-2-carboxamide,
(4S,5R)-5-((dimethylamino)methyl)-2-((R)-1-hydroxypropan-2-yl)-4-methyl-8-(pyridin-2-ylethynyl)-2,3,4,5-tetrahydrobenzo[b][1,4,5]oxathiazocine 1,1-dioxide,
1-(3,3a,4,5,6,6a-hexahydro-1H-cyclopenta[c]pyrrol-2-yl)-3-(4-methylphenyl)sulfonylurea,
1-[(3,4-dimethoxyphenyl)methyl]-6,7-dimethoxyisoquinoline,
1-[(4R,7S,8S)-8-methoxy-4,7,10-trimethyl-11-oxo-2-oxa-5,10-diazabicyclo[10.4.0]hexadeca-1(12),13,15-trien-14-yl]-3-[4-(trifluoromethyl)phenyl]urea,
1-[[(8S,9R)-6-[(2S)-1-hydroxypropan-2-yl]-8-methyl-5-oxo-10-oxa-1,6,14,15-tetrazabicyclo[10.3.0]pentadeca-12,14-dien-9-yl]methyl]-1-methyl-3-(3-pyridin-2-yloxyphenyl)urea,
2-[(1R,3R,4aS,9aR)-1-(hydroxymethyl)-6-[(3-methoxyphenyl)sulfonylamino]-3,4,4a,9a-tetrahydro-1H-pyrano[3,4-b]benzofuran-3-yl]acetic acid methyl ester,
2-[(2R,3R,6S)-3-[[(2,5-difluoroanilino)-oxomethyl]amino]-2-(hydroxymethyl)-3,6-dihydro-2H-pyran-6-yl]-N-[3-(4-morpholinyl)propyl]acetamide,
2-[(3 S,6aR,8R,10aR)-1-(1,3-benzodioxol-5-ylmethyl)-3-hydroxy-3,4,6,6a,8,9,10,10a-octahydro-2H-pyrano[2,3-c][1,5]oxazocin-8-yl]-1-piperidin-1-ylethanone,
2-[(3 S,6aR,8S,10aR)-3-hydroxy-1-(3-methoxyphenyl)sulfonyl-3,4,6,6a,8,9,10,10a-octahydro-2H-pyrano[2,3-c][1,5]oxazocin-8-yl]-1-(4-phenyl-1-piperazinyl)ethanone1-pyridin-4-yl-3-(2,4,6-trichlorophenyl)urea,
4-(5,7,7,10,10-pentamethyl-8,9-dihydronaphtho[2,3-b][1,4]benzodiazepin-13-yl)benzoic acid (LE-135),
4-fluoro-N-[(2R,3R)-5-[(2R)-1-hydroxypropan-2-yl]-3-methyl-2-(methylaminomethyl)-6-oxo-3,4-dihydro-2H-1,5-benzoxazocin-10-yl]benzenesulfonamide,
5-(1,4-diazepan-1-ylsulfonyl)-2H-isoquinolin-1-one,
5-(4-chlorophenyl)-6-ethylpyrimidine-2,4-diamine,
5-amino-1-[2,6-dichloro-4-(trifluoromethyl)phenyl]-4-(trifluoromethane)sulfinyl-1H-pyrazole-3-carbonitrile,
7-hydroxy-3-(4-hydroxyphenyl)chromen-4-one,
N-[(1R,3R,4aS,9aR)-3-[2-[(3-fluorophenyl)methylamino]-2-oxoethyl]-1-(hydroxymethyl)-3,4,4a,9a-tetrahydro-1H-pyrano[3,4-b]benzofuran-6-yl]-1,3-benzodioxole-5-carboxamide,
N-[(1S,3S,4aR,9aS)-1-(hydroxymethyl)-3-[2-oxo-2-(1-piperidinyl)ethyl]-3,4,4a,9a-tetrahydro-1H-pyrano[3,4-b]benzofuran-6-yl]-4-oxanecarboxamide,
N-[(1S,3S,4aS,9aR)-1-(hydroxymethyl)-3-[2-oxo-2-(pyridin-2-ylmethylamino)ethyl]-3,4,4a,9a-tetrahydro-1H-pyrano[3,4-b][1]benzofuran-6-yl]cyclobutanecarboxamide,
N-[(2R,3 S,6S)-6-[2-[(4-fluorophenyl)sulfonylamino]ethyl]-2-(hydroxymethyl)oxan-3-yl]oxane-4-carboxamide,
N-[(2S,3S,6R)-2-(hydroxymethyl)-6-[2-oxo-2-(1,3-thiazol-2-ylamino)ethyl]oxan-3-yl]-3-piperidin-1-ylpropanamide,
N-[(4R,7S,8R)-8-methoxy-4,7,10-trimethyl-11-oxo-5-(1,3-thiazol-2-ylmethyl)-2-oxa-5,10-diazabicyclo[10.4.0]hexadeca-1(12),13,15-trien-14-yl]cyclohexanecarboxamide,
N-[(4S,7R,8R)-8-methoxy-4,7,10-trimethyl-11-oxo-5-(phenylmethyl)-2-oxa-5,10-diazabicyclo[10.4.0]hexadeca-1(12),13,15-trien-14-yl]butanamide,
N-[(4S,7S,8S)-8-methoxy-4,7,10-trimethyl-11-oxo-2-oxa-5,10-diazabicyclo[10.4.0]hexadeca-1(12),13,15-trien-15-yl]-4-phenylbenzamide,
N-[(5S,6S,9S)-8-(cyclopropylmethyl)-5-methoxy-3,6,9-trimethyl-2-oxo-11-oxa-3,8-diazabicyclo[10.4.0]hexadeca-1(12),13,15-trien-14-yl]-2-fluorobenzamide,
N-[[(8R,9S)-6-[(2R)-1-hydroxypropan-2-yl]-8-methyl-5-oxo-10-oxa-1,6,13,14 tetrazabicyclo[10.2.1]pentadeca-12(15),13-dien-9-yl]methyl]-N-methyl-4 phenoxybenzenesulfonamide,
N-benzyl-2-chloroquinazolin-4-amine, and
N-benzylquinazolin-4-amine; or
a pharmaceutically acceptable salt thereof, or a tautomer, stereoisomer, prodrug and/or solvate of any of the foregoing.

15. The method of claim 14, wherein the is selected from the group consisting of N-benzylquinazolin-4-amine, N-[(4S,7S,8S)-8-methoxy-4,7,10-trimethyl-11-oxo-2-oxa-5,10-diazabicyclo[10.4.0]hexadeca-1(12),13,15-trien-15-yl]-4-phenylbenzamide, and 1-[(4R,7S,8S)-8-methoxy-4,7,10-trimethyl-11-oxo-2-oxa-5,10-diazabicyclo[10.4.0]hexadeca-1(12),13,15-trien-14-yl]-3-[4-(trifluoromethyl)phenyl]urea; or
a pharmaceutically acceptable salt thereof, or a tautomer, stereoisomer, prodrug and/or solvate of any of the foregoing.

16. The method of claim 15, the method comprising administering to the subject N-benzylquinazolin-4-amine.

17. The method of claim 14, wherein the neoplasia is selected from the group consisting of sarcoma, bladder cancer, pancreatic cancer, prostate cancer, head and neck cancer, breast cancer, and liver cancer.

18. The method of claim 17, wherein the neoplasia is undifferentiated pleomorphic sarcoma.

19. The method of claim 14, wherein said neoplasia is associated with the NF-κB pathway.

20. The method of claim 14, wherein said neoplasia is associated with the Hippo pathway.


SEQUENCE LISTING (14 sequences)

SEQ ID NO: 1 - protein, 504 aa, Homo sapiens
MDPGQQPPPQPAPQGQGQPPSQPPQGQGPPSGPGQPAPAATQAAPQAPPAGHQIVHVRGDSETDLEALFNAVMNPKTANVPQTVPMRLRKLPDSFFKPPEPKSHSRQASTDAGTAGALTPQHVRAHSSPASLQLGAVSPGTLTPTGVVSGPAATPTAQHLRQSSFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQAMRNINPSTANSPKCQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 2 - protein, 292 aa, Homo sapiens
MPLHVKWPFPAVPPLTWTLASSVVMGLVGTYSCFWTKYMNHLTVHNREVLYELIEKRGPATPLITVSNHQSCMDDPHLWGILKLRHIWNLKLMRWTPAAADICFTKELHSHFFSLGKCVPVCRGAEFFQAENEGKGVLDTGRHMPGAGKRREKGDGVYQKGMDFILEKLNHGDWVHIFPEGKVNMSSEFLRFKWGIGRLIAECHLNPIILPLWHVGMNDVLPNSPPYFPRFGQKITVLIGKPFSALPVLERLRAENKSAVEMRKALTDFIQEEFQHLKTQAEQLHNHLQPGR

SEQ ID NO: 3 - DNA, 21 bp, Artificial Sequence
  Description of Artificial Sequence Synthetic primer
gtggtctcctctgacttcaac

SEQ ID NO: 4 - DNA, 21 bp, Artificial Sequence
  Description of Artificial Sequence Synthetic primer
cctgttgctgtagccaaattc

SEQ ID NO: 5 - DNA, 20 bp, Artificial Sequence
  Description of Artificial Sequence Synthetic primer
gctgccaccaagctagataa

SEQ ID NO: 6 - DNA, 22 bp, Artificial Sequence
  Description of Artificial Sequence Synthetic primer
gtgcatgtgtctccttagatcc

SEQ ID NO: 7 - DNA, 19 bp, Artificial Sequence
  Description of Artificial Sequence Synthetic primer
gtgcatccgtactcccaaa

SEQ ID NO: 8 - DNA, 23 bp, Artificial Sequence
  Description of Artificial Sequence Synthetic primer
ctccacagaatttagctcggtat

SEQ ID NO: 9 - DNA, 21 bp, Artificial Sequence
  Description of Artificial Sequence Synthetic primer
agcctcgcatcctatacaacc

SEQ ID NO: 10 - DNA, 21 bp, Artificial Sequence
  Description of Artificial Sequence Synthetic primer
ttctttcacaaggcggcactc

SEQ ID NO: 11 - DNA, 21 bp, Artificial Sequence
  Description of Artificial Sequence Synthetic primer
gatgtctcaggaattgagaac

SEQ ID NO: 12 - DNA, 21 bp, Artificial Sequence
  Description of Artificial Sequence Synthetic primer
ctgtatccatttcatccacac

SEQ ID NO: 13 - DNA, 21 bp, Artificial Sequence
  Description of Artificial Sequence Synthetic primer
ctgcgctaaacaactcaacga

SEQ ID NO: 14 - DNA, 20 bp, Artificial Sequence
  Description of Artificial Sequence Synthetic primer
gcagatccctttcagagcgg